SEQUENCE LISTING (2 sequences)

SEQ ID NO: 1 - protein, 8 aa, Artificial Sequence
  Ovalbumin peptide
SIINFEKL

SEQ ID NO: 2 - protein, 9 aa, Artificial Sequence
  Ovalbumin peptide
AAHAEINEA

(19) United States
(12) Patent Application Publication
(10) Pub. No.: US 2023/0076515 A1
(43) Pub. Date: Mar. 9, 2023

(54) STIMULI THAT HYPERACTIVATE RESIDENT DENDRITIC CELLS FOR CANCER IMMUNOTHERAPY

(71) Applicant: Children's Medical Center Corporation, Boston, MA (US)

(72) Inventors: Jonathan C. Kagan, Brookline, MA (US); Dania Zhivaki, Boston, MA (US)

(21) Appl. No.: 17/777,659

(22) PCT Filed: Nov. 18, 2020
(86) PCT No.: PCT/US20/61133

§ 371 Date: May 18, 2022

Related U.S. Application Data
(60) Provisional application No. 62/937,073, filed on Nov. 18, 2019.

(51) Int. Cl. A61K 39/00; A61K 39/39; A61P 35/00

(52) U.S. Cl. CPC A61K 39/0011; A61K 39/39; A61P 35/00; A61K 2039/55572; A61K 2039/55577

(57) ABSTRACT

The present application is related to cancer immunotherapy, e.g. stimulation of T cell mediated anti-tumor therapy. Accordingly, described herein are methods of inducing or enhancing an adaptive immune response to a cancer in a subject and methods of treating cancer in a subject. In some embodiments, the methods hyperactivate dendritic cells (DCs), which induce T helper type I (TH1) and cytotoxic T lymphocyte (CTL) responses in the absence of TH2 immunity.

### CLAIM OF PRIORITY

This application claims the benefit of U.S. Provisional Application Ser. No. 62/937,073, filed on Nov. 18, 2019. The entire contents of the foregoing are incorporated herein by reference.

### STATEMENT REGARDING FEDERALLY SPONSORED RESEARCH

This invention was made with government support under Grant No.: AI116550 awarded by the National Institutes of Health. The government has certain rights in the invention.

### TECHNICAL FIELD

The present application is related to cancer immunotherapy, e.g. stimulation of T cell mediated anti-tum20aor therapy.

### BACKGROUND

Central to the understanding of protective immunity to infection and cancer are dendritic cells (DCs), which are migratory phagocytes that patrol the tissues of the body (D. Alvarez, E. H. et al., Immunity, vol. 29, no. 3, pp. 325-42, September 2008). DCs survey the environment for threats to the host, most commonly infection or evidence of tissue damage. This surveillance is achieved through the actions of a superfamily of threat assessment receptors (classically known as pattern recognition receptors (PRRs), which recognize microbial products or host-encoded molecules indicative of tissue injury (S. W. Brubaker, et al. Annu. Rev. Immunol., vol. 33, pp. 257-90, 2015; C. A. Janeway and R. Medzhitov, Annu. Rev. Immunol., vol. 20, pp. 197-216, January 2002). Microbial ligands for PRRs are classified as pathogen associated molecular patterns (PAMPs) whereas host-derived PRR ligands are damage associated molecular patterns (DAMPs) (P. Matzinger, Science, vol. 296, no. 5566, pp. 301-5, April 2002).

Upon detection of PAMPs, PRRs unleash signaling pathways that fundamentally alter the physiology of the DCs that express these receptors (A. Iwasaki and R. Medzhitov, Nat. Immunol., vol. 16, no. 4, pp. 343-53, April 2015; O. Joffre, et al. Immunol. Rev., vol. 227, no. 1, pp. 234-247, January 2009). For example, prior to PRR activation, DCs are typically viewed as non-inflammatory cells. Upon encountering extracellular PAMPs, PRRs stimulate the rapid and robust upregulation of numerous inflammatory mediators, including cytokines, chemokines and interferons. Co-incident with the expression of these genes is the migration of DCs to draining lymph nodes (dLN) and the upregulation of factors important for T cell activation, such as MHC and co-stimulatory molecules. Thus, the process of PRR signaling leads to a shift in DC activities from a non-stimulatory (naïve) state to an “activated” state (K. Inaba, et al. J. Exp. Med., vol. 191, no. 6, pp. 927-36, March 2000 I. Mellman and R. M. Steinman, Cell, vol. 106, no. 3, pp. 255-8, August 2001).

### SUMMARY

There is a need to diversify current approaches to cancer immunotherapy. Accordingly, described herein are methods of inducing or enhancing an adaptive immune response to a cancer in a subject and methods of treating cancer in a subject. In some embodiments, the methods hyperactivate dendritic cells (DCs), which induce T helper type I (TH1) and cytotoxic T lymphocyte (CTL) responses in the absence of TH2 immunity. Hyperactivating stimuli drive T cell responses that protect against tumors that are sensitive or resistant to PD-1 inhibition. These protective responses depend on inflammasomes in DCs and can be generated using tumor lysates as immunogens.

Thus, provided herein are methods of inducing or enhancing an adaptive immune response to a cancer in a subject that comprise administering an effective amount of (i) a Toll-Like Receptor (TLR) ligand; (ii) a non-canonical inflammasome-activating lipid; and (iii) an cancer immunogen to the subject.

Also provided herein are methods of treating cancer in a subject that comprise administering an effective amount of (i) a Toll-Like Receptor (TLR) a TLR ligand; (ii) a non-canonical inflammasome-activating lipid, and (iii) a cancern immunogen to the subject.

In some embodiments of the methods described herein, the cancer immunogen is an infectious agent immunogen, wherein an infection with the infectious agent is associated with development of cancer.

In some embodiments of the methods described herein, the cancer immunogen is from a cancer immunogen cell.

In some embodiments of the methods described herein, the cancer immunogen is or comprises whole tumor cell lysate.

In some embodiments of the methods described herein, the TLR ligand is selected from a TLR1 ligand, a TLR2 ligand, a TLR3 ligand, a TLR4 ligand, a TLR5 ligand, a TLR6 ligand, a TLR7 ligand, a TLR8 ligand, a TLR9 ligand, a TLR10 ligand, a TLR11 ligand, a TLR12 ligand, a TLR13 ligand, and combinations thereof.

In some embodiments of the methods described herein, the TLR ligand is a TLR4 ligand.

In some embodiments of the methods described herein, the TLR4 ligand is selected from monophosphoryl lipid A (MPLA), lipopolysaccharide (LPS), or combinations thereof.

In some embodiments of the methods described herein, the non-canonical inflammasome-activating lipid comprises a species of oxidized 1-palmitoyl-2-arachidonoyl-sn-glycero-3-phosphorylcholine (oxPAPC).

In some embodiments of the methods described herein, the non-canonical inflammasome-activating lipid comprises 2-[[(2R)-2-[(E)-7-carboxy-5-hydroxyhept-6-enoyl]oxy-3-hexadecanoyloxypropoxy]-hydroxyphosphoryl]oxyethyl-trimethylazanium (HOdiA-PC), [(2R)-2-[(E)-7-carboxy-5-oxohept-6-enoyl]oxy-3-hexadecanoyloxypropyl] 2-(trimethylazaniumyl)ethyl phosphate (KOdiA-PC), 1-palmitoyl-2-(5-hydroxy-8-oxo-octenoyl)-sn-glycero-3-phosphorylcholine (HOOA-PC), 2-[[(2R)-2-[(E)-5,8-dioxooct-6-enoyl]oxy-3-hexadecanoyloxypropoxy]-hydroxyphosphoryl]oxyethyl-trimethylazanium (KOOA-PC), [(2R)-3-hexadecanoyloxy-2-(5-oxopentanoyloxy)propyl] 2-(trimethylazaniumyl)ethyl phosphate (POVPC), [(2R)-2-(4-carboxybutanoyloxy)-3-hexadecanoyloxypropyl] 2-(trimethylazaniumyl)ethyl phosphate (PGPC), [(2R)-3-hexadecanoyloxy-2-[4-[3-[(E)-[2-[(Z)-oct-2-enyl]-5-oxocyclopent-3-en-1-ylidene]methyl]oxiran-2-yl]butanoyloxy]propyl] 2-(trimethylazaniumyl)ethyl phosphate (PECPC), [(2R)-3-hexadecanoyloxy-2-[4-[3-[(E)-[3-hydroxy-2-[(Z)-oct-2-enyl]-5-oxocyclopentylidene]methyl]oxiran-2-yl]butanoyloxy]propyl] 2-(trimethylazaniumyl)ethyl phosphate (PEIPC), or a combination thereof.

In some embodiments of the methods described herein, the non-canonical inflammasome-activating lipid comprises [(2R)-2-(4-carboxybutanoyloxy)-3-hexadecanoyloxypropyl] 2-(trimethylazaniumyl)ethyl phosphate (PGPC).

In some embodiments of the methods described herein, the subject is a mammal.

In some embodiments of the methods described herein, the subject is a human.

In some embodiments of the methods described herein, the TLR ligand, oxPAPC species, and cancer immunogen are administered as part of a pharmaceutical composition.

In some embodiments of the methods described herein, the immune response is a prophylactic immune response.

In some embodiments of the methods described herein, the immune response is a therapeutic immune response.

In some embodiments of the methods described herein, the adaptive immune response comprises T-cell activation.

In some embodiments of the methods described herein, the method further comprises treating the subject with one or more therapeutic interventions.

In some embodiments of the methods described herein, the TLR ligand, oxPAPC species, and cancer immunogen, and one or more therapeutic interventions are co-administered or sequentially administered.

In some embodiments of the methods described herein, the one or more therapeutic interventions comprises: radiation, chemotherapy, surgery, therapeutic antibodies, immunomodulatory agents, proteasome inhibitors, pan-deacetylase (DAC) inhibitors, histone deacetylase (HDAC) inhibitors, checkpoint inhibitors, adoptive cell therapies, vaccines or combinations thereof.

In some embodiments of the methods described herein, the adoptive cell therapies comprise: CAR-T cell therapy, CAR-NK cell therapy, T cells, dendritic cells or combinations thereof. Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

FIGS. 1A-1H are a series of graphs demonstrating that hyperactive DCs are superior antigen-presenting cells and drive TH1-skewed immune responses, with no evidence of TH2 immunity. FIGS. 1A-1F: WT BMDCs were either left untreated (none) or treated with LPS alone, or Alum alone, or oxPAPC or PGPC alone for 24 h, or BMDCs were primed for 3 h with LPS, then treated with indicated stimuli for 21 h. FIG. 1A: IL-1β, and TNFα cytokine release was monitored by ELISA. FIG. 1B: Percentage of cell death was measured by LDH release in the cell supernatants. FIG. 1C: BMDCs treated with indicated stimuli as in FIG. 1A, were stained with live-dead violet kit, CD11c and CD40. The Mean fluorescence intensity (MFI) of surface CD40 (among CD11c⁺ live cells) is measured by flow cytometry. FIG. 1D: BMDCs pretreated with indicated stimuli were transferred onto CD40-coated plates and cultured for 24 h. IL-12p70 cytokine release was measured by ELISA. FIG. 1E: BMDCs pretreated with indicated stimuli as in FIG. 1A, were incubated with OVA protein for 2 h or FITC labeled-OVA for 45 minutes. OVA-FITC uptake (left panel) was assessed by flow cytometry. Data are represented as the percentage of OVA-associated CD11c⁺ BMDCs at 37° C. and normalized to OVA-associated CD11c⁺ BMDCs at 4° C. OVA peptide presentation on MHC-I (right panel) was monitored using PE-conjugated antibody to H-2Kb bound to the OVA peptide SIINFEKL (SEQ ID NO: 1). Data are represented as the frequency of SIINFEKL (SEQ ID NO: 1)-associated DCs among CD11c⁺ live cells. Means and SDs from three replicates are shown and data are representative of at least three independent experiments. FIG. 1F: BMDCs treated with indicated stimuli as in FIG. 1A, were loaded (or not) with OVA protein or the OVA peptide SIINFEKL for 1 h, then incubated for 4 days with splenic OT-II naïve CD4⁺ T cells or OT-I naïve CD8⁺ T cells. FIG. 1F: Supernatants were collected at day 4 and IFNγ, IL-2, IL-1β, TNFα and IL-13 cytokine release was measured by ELISA. FIG. 1G: BMDCs were either left untreated (none), or treated with LPS for 24 h, or BMDCs were primed with LPS for 3 h, then treated with PGPC or Alum for 21 h. Treated BMDCs were then cultured with splenic OT-I or OT-II T cells as in FIG. 1F. 4 days post-co-culture, CD4⁺ and CD8⁺ T cells were stimulated for 5 h with PMA plus ionomycin in the presence of brefeldin-A and monensin. The frequency of TH1 cells as TNFα⁺ IFNγ⁺, and TH2 cells as Gata3⁺ IL-4⁺ IL-10⁺ among CD4⁺ T cells was measured by intracellular staining. Data are represented as the ratio of TH1/TH2 cells (left panel). The frequency of IFNγ⁺ among CD8⁺ T cells is represented in the right panel. Means and SDs from three replicates are shown and each panel is representative of at least two independent experiments. FIG. 1H: C57BL/6 mice were injected subcutaneously on the right flank with endofit-OVA protein either alone or with LPS that were emulsified in either incomplete Freud's adjuvant (IFA) or in Alum as indicated. Alternatively, mice were injected with endofit-OVA protein and LPS plus OxPAPC or PGPC all emulsified in IFA. 40 days post immunization, CD4⁺ and CD8⁺ T cells were isolated from the skin draining lymph nodes (dLN). T cells were then cultured with naïve BMDC loaded (or not) with OVA or with SIINFEKL peptide for 5 days. IFNγ, IL-10, and IL-13 secretion was measured by ELISA. Means and SDs of four mice are shown and each panel is representative of two independent experiments. *P<0.05; **P<0.01; ***P<0.005.

FIGS. 2A-2D are a series of graphs demonstrating that hyperactive stimuli enhance memory T cell generation, and potentiates antigen-specific IFNγ effector responses in NLRP3-dependent manner. FIGS. 2A, 2B: C57BL/6 mice were injected subcutaneously (s.c.) on the right flank with endofit-OVA either alone or with LPS or with PGPC, or with LPS plus OxPAPC or PGPC all emulsified in incomplete Freud's adjuvant (IFA). 7 or 40 days post immunization, T cells were isolated from the skin draining lymph nodes (dLN) by magnetic enrichment using anti-CD4 and anti-CD8 beads. FIG. 1A: The percentage of T effector cells (Teff) as CD44^{low}CD62L^{low}, T effector memory cells (TEM) as CD44^{hi}CD62L^{low}, and T central memory cells (TCM) as CD44^{hi}CD62L^{hi} are represented among CD3⁺CD4⁺ live cells (left panels), or CD3⁺CD8⁺ live cells (right panels). FIG. 2B: CD8⁺ T cells were sorted from the dLN 7 days post immunization, then treated either with PMA plus ionomycin, or co-cultured with B160VA cells (target cells) at ratio of 1:3 (effector: target) for 5 h. Degranulation was assessed by monitoring the percentage of CD107a⁺ among live CD8⁺ T cells by flow cytometry. Means and SDs of five mice are shown and are representative of 3 independent experiments. FIGS. 2C-2D: C57BL/6 mice were injected s.c. on the right flank with endofit-OVA either alone or with LPS, or with LPS plus OxPAPC or PGPC all emulsified in IFA, or with LPS plus Alum. Alternatively, mice were injected with LPS plus PGPC alone (no OVA). NLRP3^{-/-} or Casp1/11^{-/-} mice were similarly injected with endo-fit-OVA with LPS plus PGPC emulsified in IFA. FIG. 2C: 7 days post-immunization, CD4⁺ and CD8⁺ T cells were sorted from the skin dLN of immunized mice, and co-cultured with BMDC loaded (or not) with OVA for 7 days at a ratio of 1:10 (DC: T cell). The percentage of SIINFEKL⁺ (SEQ ID NO: 1) IFNγ⁺ among CD8⁺ live T cells (upper panel), and AAHAEINEA⁺ (SEQ ID NO: 2) IFNγ⁺ among CD4⁺ live T cells (lower panel) were measured using OVA peptides tetramer staining and intracellular IFNγ staining. FIG. 2D: The percentage of CD44⁺ memory T cells in the inguinal adipose tissue was assessed by flow cytometry. Means and SDs of five mice are shown. *P<0.05; **P<0.01; ***P<0.005, ****P<0.0005.

FIGS. 3A-3J are a series of graphs and plots demonstrating that hyperactive DCs induce inflammasome-dependent anti-tumor immunity. FIGS. 3A, 3B: C57BL/6 mice were injected subcutaneously (s.c.) on the right flank with PBS (unimmunized), B160VA cell lysate alone (none) or with LPS, or B16OVA lysate plus LPS and OxPAPC or PGPC all emulsified in incomplete Freud's adjuvant (IFA). 15 days post immunization, mice were challenged s.c. on the left upper back with 3×10⁵ of viable B160VA cells. 150 days later, tumor-free mice were re-challenged s.c. with 5×10⁵ of viable B16OVA cells on the back. FIG. 3A: Tumor growth was monitored every 2 days (upper panel). For survival experiments (lower panel), mice were allowed to reach 20 mm of diameter (n=8-15 mice per group). FIG. 3B: Tumors were harvested at the endpoint of tumor growth, and dissociated to obtain single-cell tumor suspension. The percentage of tumor infiltrating CD3⁺CD4⁺ and CD3⁺CD8⁺ T cells among enriched CD45⁺ live cells was assessed by flow cytometry (upper panel). Tumor infiltrating CD3⁺ T cells were sorted then stimulated for 24 h in the presence of anti-CD3 and anti-CD28 DYNABEADS™ (IhermoFisher Scientific). IFNγ release was measured by ELISA (lower panel) (n=4 mice per group). FIG. 3C: Absolute number of CD8⁺ T cells (upper panel) and CD69⁺ CD103⁺ T resident memory CD8⁺ T cells (lower panel) was assessed at the immunization or tumor injection site of survivor mice from FIG. 3A, and measured by flow cytometry (n=4 mice). FIG. 3D: Absolute number of total CD8⁺ T cells (upper panels), SIINFEKL⁺ (SEQ ID NO: 1) (middle panels) and CD69⁺ CD103⁺ (lower panels) among CD8⁺ T cells in the spleen (left panels) or the skin inguinal adipose tissue (right panels) of survivor mice or age-matched unimmunized tumor-bearing mice (n=5 mice per group). FIGS. 3E-3F: Circulating memory CD8⁺ T cells (TCM) were isolated from the spleen, and T resident memory CD8⁺ cells (TRM) were isolated from the skin inguinal adipose tissue of survivor mice or age-matched unimmunized tumor-bearing mice. FIG. 3E: TCM and TRM from survivor mice were co-cultured with B16OVA or B16-F10 or CT26 tumor cells for 5 h at a ratio of 1:5 (tumor cell: T cell). Cell death by cytolytic CD8⁺ T cells was measured by LDH release in the supernatants. FIG. 3F: C57BL/6 recipient mice were either left untreated (no Tx), or were inoculated intravenously (i.v.) with 5×10⁵ of CD8⁺ TCM cells and/or intradermally (i.d.) with 5×10⁵ of CD8⁺ TRM cells isolated from survivor mice or age-matched unimmunized tumor-bearing mice. 7 days later, all mice were challenged with 3×10⁵ of viable B16OVA cells. Percentage of survival was monitored every 2 days. Mice were allowed to reach 20 mm of diameter (n=5 mice per group). FIG. 3G: C57BL/6 mice were either left untreated (unimmunized), or were immunized s.c. on the right flank with B16OVA tumor lysate alone or with MPLA, or with B16OVA lysate and MPLA plus PGPC with/o neutralizing anti-mouse IL-1β antibody. 15 days post immunization, mice were challenged with 3×10⁵ of viable B16OVA cells s.c. on the left upper back. 90 days later, tumor-free mice were re-challenged with 5×10⁵ of viable B16 OVA cells s.c. on the back. Survival was monitored every 2 days (n=3-4 mice per group). FIG. 3H: WT C57BL/6, NLRP3^{-/-}, Casp11^{-/-} or Casp1/11^{-/-} mice were immunized s.c. on the right flank with B160VA cell lysate, LPS plus PGPC all emulsified in IFA. 15 days post immunization, mice were challenged with 3×10⁵ of viable B16OVA cells s.c. on the left upper back. The percentage of survival is represented (n=5 mice per group). FIGS. 3I-3J: C57BL/6 mice were either left untreated (unimmunized), or were immunized s.c. on the right flank with MC380VA lysate with MPLA, or MC380VA lysate with MPLA plus PGPC w/o i.v. injection of anti-mouse IL-1β antibody for 5 consecutive days starting two days prior the immunization. Alternatively, mice were immunized with OVA protein and MPLA plus PGPC. 15 days later, mice were inoculated s.c. with 5×10⁵ live MC380VA cells on the left upper back. 50 days later, tumor-free mice were re-challenged s.c. with 1×10⁶ MC380VA cells on the back. (I) The percentage of survival is indicated (n=5 mice per group). FIG. 3J: 170 days after the initial inoculation with MC380VA tumor cells, survivor mice or age matched naïve mice were challenged with 3×10⁵ of viable B16-F10 cells. The percentage of survival is indicated. (n=5 mice per group). *P<0.05; **P<0.01; ***P<0.005. Tx; T cell injection, WT; wild type.

FIGS. 4A-4F are a series of schematic representations and graphs demonstrating that hyperactive stimuli are potent adjuvants for cancer immunotherapy. FIG. 4A: Schematic diagram of the hyperactive-based immunotherapy approach, and legend corresponding to the neutralizing antibodies used as follow; anti IL-1β injected intravenously (i.v.), anti-CD4, anti-CD8a, and anti-PD1 antibodies were injected intraperitoneally. FIG. 4B: C57BL/6 WT mice were inoculated s.c. with 5×10⁵ of live MC380VA cells on the left upper back. 14 days later, mice were either left untreated (unimmunized) or were injected s.c. on the right flank with syngeneic MC380VA whole tumor lysate (WTL), plus LPS and PGPC with or without injection of neutralizing antibodies as indicated in the schematic diagram. Mice received 2 boost injections with WTL and LPS plus PGPC on day 37 and on day 55 post tumor inoculation. Tumors were allowed to reach 20 mm of diameter. The percentage of survival is indicated (n=10 mice per group). FIG. 4B: C57BL/6 WT mice were inoculated s.c. with 3×10⁵ live B160VA cells on the left upper back. 10 days later, mice were either left untreated (unimmunized), or injected with anti-PD1 antibody as indicated in the schematic diagram. Alternatively, mice were injected s.c. on the right flank with syngeneic B160VA WTL, plus LPS and PGPC with or without neutralizing antibodies as indicated. Mice received 2 boost injections with B160VA WTL, plus LPS and PGPC on day 17 and on day 24 post tumor inoculation. The percentage of survival is indicated (n=10 mice/group). FIG. 4C: BALB/c WT mice were inoculated s.c. with 3×10⁵ live CT26 cells on the left back. 7 days later, mice were either left untreated (unimmunized), or injected with anti-PD1 antibody as indicated in the diagram. Alternatively, mice were injected s.c. on the right flank with syngeneic CT26 WTL, plus LPS and PGPC with or without indicated neutralizing antibodies. Mice received 2 boost injections on day 14 and day 21 post tumor inoculation. The percentage of survival is indicated (n=10 mice/group). FIG. 4D: C57BL/6 WT mice were inoculated s.c. on the left upper back with 3×10⁵ live B16-F10 cells. 7 days later, mice were either left untreated (unimmunized), or injected with anti-PD1 antibody as indicated in the schematic diagram. Alternatively, mice were immunized s.c. on the right flank with syngeneic B16-F10 WTL, plus LPS and PGPC with or without indicated neutralizing antibodies. Mice received 2 boost injections on day 14 and day 21 post tumor inoculation. The percentage of survival is indicated (n=10 mice per group). FIG. 4F: C57BL/6 WT mice were inoculated i.v. with 3×10⁵ live B16-F10 cells. 5 days later, mice were either left untreated (unimmunized) or were injected with B16F-10 syngeneic WTL either alone or with LPS or with LPS plus PGPC. Metastatic nodules on the lung were counted 17 days post tumor cells inoculation (n=5 mice per group).

FIGS. 5A-5E are a series of graphs demonstrating that hyperactive DCs are superior antigen-presenting cells and drive TH1-skewed immune responses, with no evidence of TH2 immunity. FIGS. 5A, 5B: BMDC generated with GMCSF were left untreated (None), or were treated with MPLA alone, Alum alone, or OxPAPC or PGPC alone or BMDCs were primed for 3 h with MPLA, then treated with indicated stimuli for 21 h. FIG. 5A: IL-1β, and TNFα cytokine release was monitored by ELISA. FIG. 5B: Percentage of cell death was measured by LDH release in the cell supernatants. FIGS. 5C, 5D: Splenic CD11c⁺ were sorted and either left untreated (None), or were treated either with LPS alone, or Alum alone, or PGPC alone or DCs were primed for 3 h with LPS, then treated with indicated stimuli for 21 h. FIG. 5C: IL-1β, and TNFα cytokine release was monitored by ELISA. FIG. 5D: Percentage of cell death was measured by LDH release in the cell supernatants. FIGS. 5E-5F: BMDCs generated with GMCSF treated with indicated stimuli as in A, were stained with live-dead violet kit, anti-CD11c, anti-CD80, anti CD69, and anti-H2kb antibodies. FIG. 5E: The Mean fluorescence intensity (MFI) of surface CD80 (left panel) and CD69 (middle panel) and H2Kb (right panel) among CD11c⁺ live cells was measured by flow cytometry. Means and SDs of three replicates are shown and all panels are representative of at least three independent experiments. *P<0.05.

FIGS. 6A-6C are a graph and a series of plots demonstrating that hyperactive DCs are superior antigen-presenting cells and drive TH1-skewed immune responses, with no evidence of TH2 immunity. FIGS. 6A-6C: WT BMDCs were either left untreated (none) or treated with LPS alone, or Alum alone, or OxPAPC or PGPC alone for 24 h, or BMDCs were primed for 3 h with LPS, then treated with indicated stimuli for 21 h. FIG. 6A: BMDCs were incubated with fixable FITC labeled-OVA at 37° C. or at 4° C. for 45 minutes. BMDCs were then stained with live-dead violet kit. Gating strategy to determine the frequency of OVA-FITC associated BMDCs at 37° C. as compared to OVA-associated BMDCs at 4° C. by flow cytometry. FIG. 6B: BMDCs were incubated with endofit-OVA protein for 2 hours. Gating strategy to determine the frequency of SIINFEKL (SEQ ID NO: 1) peptide bound to H2kb on the surface of live BMDCs as measured by flow cytometry using PE-conjugated antibody to H-2Kb bound to the OVA peptide SIINFEKL. Each panel is representative of three replicates of one out of three experiments. FIG. 6C: C57BL/6 mice were injected subcutaneously on the right flank with endofit-OVA protein either alone or with LPS that were emulsified in either incomplete Freud's adjuvant (IFA) or in Alum as indicated. Alternatively, mice were injected with endofit-OVA protein and LPS plus OxPAPC or PGPC all emulsified in IFA. 40 days post immunization, CD4⁺ T cells were isolated from the skin draining lymph nodes (dLN). T cells were then cultured with naïve BMDC loaded (or not) with OVA for 5 days. IL-4 secretion was measured by ELISA. Means and SDs of four mice are shown and each panel is representative of two independent experiments ***P<0.005.

FIG. 7 is a series of plots demonstrating that hyperactive DCs are superior antigen-presenting cells and drive TH1-skewed immune responses, with no evidence of TH2 immunity. BMDCs were either left untreated (none), or treated with LPS for 24 h, or BMDCs were primed with LPS for 3 h, then treated with PGPC or Alum for 21 h. Treated BMDCs were then cultured with splenic OT-II T cells at a ratio of 1:5 (BMDC: T cell). 4 days post-co-culture, CD4⁺ T cells were stimulated for 5 h with PMA plus ionomycin in the presence of brefeldin-A and monensin. Gating strategy to determine the frequency of TH2 cells as IL-4⁺ IL-10⁺ among live CD4⁺ T cells as measured by intracellular staining. Each panel is representative of three replicates of one out of three experiments.

FIGS. 8A-8E are a series of plots and graphs demonstrating that hyperactive stimuli enhance memory T cell generation, and potentiates antigen-specific IFNγ effector responses in NLRP3-dependent manner. C57BL/6 mice were injected subcutaneously (s.c.) on the right flank with endofit-OVA either alone or with LPS or with PGPC, or with LPS plus OxPAPC or PGPC all emulsified in incomplete Freud's adjuvant (IFA). 7 days post immunization, T cells were isolated from the skin draining lymph nodes (dLN) by magnetic enrichment using anti-CD4 and anti-CD8 beads. FIG. 8A: Gating strategy to determine the percentage of T effector cells (Teff) as CD44^{low}CD62L^{low}, T effector memory cells (TEM) as CD44^{hi}CD62L^{low}, and T central memory cells (TCM) as CD44^{hi}CD62L^{hi} among CD3⁺CD4⁺ live cells. FIG. 8B: Absolute number of Teff or TEM cells in the skin dLN per mouse was assessed by flow cytometry among total CD3⁺ live cells. FIG. 8C: Sorting strategy and post-sorting purity is represented for CD4⁺ and CD8⁺ T cell subsets. FIG. 8D: CD4⁺ and CD8⁺ T cells were sorted from the dLN 7 days post immunization, then cultured with BMDC loaded (or not) with a serial dilution of OVA protein starting from 1000 μg/ml. IFNγ cytokine secretion was measured by ELISA. Means and SDs of four replicates are shown. FIG. 8E: CD8⁺ T cells were sorted from the dLN 7 days post immunization, then treated either with PMA plus ionomycin, or co-cultured with B160VA cells (target cells) at ratio of 1:3 (effector: target) for 5 h. Gating strategy to determine the percentage of CD107a⁺ among live CD8⁺ T cells by flow cytometry. Each panel is representative of five replicates.

FIGS. 9A-9F are a series of graphs demonstrating that hyperactive DCs induce inflammasome-dependent anti-tumor immunity. FIGS. 9A, 9B: C57BL/6 mice were injected subcutaneously (s.c.) on the right flank with PBS (unimmunized), B160VA cell lysate alone (none) or with LPS, or B160VA lysate plus LPS and OxPAPC or PGPC all emulsified in incomplete Freud's adjuvant (IFA). 15 days post immunization, mice were challenged s.c. on the left upper back with 3×10⁵ of viable B160VA cells. 150 days later, tumor-free mice were re-challenged s.c. with 5×10⁵ of viable B160VA cells on the back. FIG. 9A: Percentage of tumor-Free mice 300 days post tumor inoculation is indicated (n=8-15 mice per group). FIG. 9B: C57BL/6 mice were injected subcutaneously (s.c.) on the right flank with PBS (unimmunized), B16-F10 cell lysate alone (none) or with LPS, or B16-F10 lysate plus LPS and PGPC all emulsified in incomplete Freud's adjuvant (IFA). 15 days post immunization, mice were challenged s.c. on the left upper back with 3×10⁵ of viable B16-F10 cells. The percentage of survival is indicated in the left panel and the percentage of tumor-Free mice 70 days post tumor inoculation is indicated in the right panel (n=5 mice per group). FIG. 9C: C57BL/6 mice were either left untreated (unimmunized), or were immunized s.c. on the right flank with B16OVA tumor lysate alone or with MPLA, or with B16OVA lysate and MPLA plus PGPC with/o neutralizing anti-mouse IL-1β antibody. 15 days post immunization, mice were challenged with 3×10⁵ of viable B16OVA cells s.c. on the left upper back. The percentage of tumor-Free mice 150 days post tumor inoculation is indicated in the right panel (n=3-4 mice per group). FIG. 9D: C57BL/6 mice were either left untreated (unimmunized), or were immunized s.c. on the right flank with MC380VA lysate with MPLA, or MC380VA lysate with MPLA plus PGPC w/o i.v. injection of anti-mouse IL-1β antibody for 5 consecutive days starting two days prior the immunization. Alternatively, mice were immunized with OVA protein and MPLA plus PGPC. 14 days later, mice were inoculated s.c. with 5×10⁵ live MC380VA cells on the left upper back. 50 days later, tumor-free mice were re-challenged s.c. with 1×10⁶ MC380VA cells on the back. The percentage of tumor-Free mice 150 days post tumor inoculation is indicated (n=5 mice per group). FIG. 9E: C57BL/6 mice were either left untreated (unimmunized) or were immunized s.c. on the right flank with B16OVA lysate (WTL) plus LPS and PGPC emulsified in IFA, or B16OVA WTL plus LPS and alum. 15 days post immunization, mice were challenged with 3×10⁵ of viable B160VA cells s.c. on the left upper back. The percentage of survival is monitored every 2 days (n=5 mice per group). FIG. 9F: C57BL/6 mice were immunized s.c. on the right flank with LPS and PGPC emulsified in IFA without any antigen. 15 days or B16OVA WTL plus LPS and alum. 15 days post immunization, mice were challenged s.c. on the left upper back with either 3×10⁵ of viable B16OVA cells, or 3×10⁵ of viable B16-F10 cells, or 5×10⁵ of viable MC380VA cells. The percentage of survival is monitored every 2 days (n=5 mice per group).

FIGS. 10A-10C are plots demonstrating that hyperactive DCs induce inflammasome-dependent anti-tumor immunity. FIG. 10A: Gating strategy to determine the absolute number of CD8⁺ T cells and CD69⁺ CD103⁺ T resident memory CD8⁺ T cells at the immunization or tumor injection site of survivor mice that were previously immunized with B160VA WTL and LPS plus PGPC (from FIG. 3A), as measured by flow cytometry. Panels are representative of four replicates. FIG. 10B: Gating strategy to determine the absolute number of total CD8⁺ T cells SIINFEKL⁺ (SEQ ID NO: 1) among CD45⁺ live cells in the skin inguinal adipose tissue from survivor mice previously immunized with B160VA WTL and LPS plus PGPC (from FIG. 3A), or age-matched unimmunized tumor-bearing mice. Panels are representative of five replicates. FIG. 10C: Gating strategy to determine the absolute number of CD8⁺ T cells and CD69⁺ CD103⁺ T resident memory CD8⁺ T cells the skin inguinal adipose tissue of survivor mice that were previously immunized with B160VA WTL and LPS plus PGPC (from FIG. 3A), as compared to age-matched unimmunized tumor-bearing mice (Panels are representative of five replicates).

FIGS. 11A-11E are a series of graphs and a schematic representation demonstrating that hyperactive DCs induce inflammasome-dependent anti-tumor immunity. C57BL/6 mice were either left untreated (unimmunized), or were immunized s.c. on the right flank with MC380VA lysate with MPLA, or MC380VA lysate with MPLA plus PGPC w/o i.v. injection of anti-mouse IL-1β antibody for 5 consecutive days starting two days prior the immunization. Alternatively, mice were immunized with OVA protein and MPLA plus PGPC. 15 days later, each group was randomly separated into two sister cohorts. FIG. 11A: Schematic representation of the experimental model. FIGS. 11B-11E) In one cohort of mice, skin draining lymph nodes (dLN) were dissected. FIG. 11B: Absolute number of total CD8⁺ T cells (left panel) and SIINFEKL⁺ (SEQ ID NO: 1) CD8⁺ T cells (right panel) in the dLN of immunized mice. (FIGS. 11C-11D: CD8⁺ T cells were enriched from the dLN using anti-CD8 magnetic beads, then CD3⁺CD8⁺ live cells were sorted. FIG. 11C: CD8⁺ T cells were cultured for 5 h with MC380VA cells (target cells). Cytotoxic CD8⁺ T cells response was monitored by CD107a degranulation assay by flow cytometry. FIG. 11D: CD8⁺ T cells were co-cultured for 4 days with naïve BMDCs loaded (or not) with a serial dilution of OVA protein starting from 1000 μg/ml. IFNγ release was monitored by ELISA. Means and SDs of five mice are shown and each panel is representative of two independent experiments.*P<0.05; **P<0.01; ***P<0.005.

FIGS. 12A and 12B are a series of graphs and a schematic demonstrating hyperactive cDC1 control tumor rejection induced by hyperactivation-based immunotherapy. (FIGS. 12A-12B) C57BL/6 WT mice or Batf3^{-/-} mice were inoculated with 3×10⁵ live B16OVA cells subcutaneously (s.c.) on the left back. 10 days later, mice were either left untreated (unimmunized) or were immunized s.c. on the right flank with syngeneic B16OVA tumor lysate, plus LPS and PGPC. Mice received 2 boost injections on day 17 and day 24 post tumor inoculation as indicated in the schematic diagram. Tumors were allowed to reach 20 mm of diameter. (FIG. 12A) The percentage of survival is indicated (n=5 mice/group). (FIG. 12B) skin draining lymph node (dLN), tumor, and spleen tissues were dissected from immunized mice 15 days post tumor inoculation. The percentage of antigen specific CD8⁺ and CD4⁺ T cells was measured using SIINFEKL and AAHAEINEA tetramer staining respectively (n=5 mice/group).

FIGS. 13A-13D show results from the mass spectrometry of synthesized lipids. Mass spectrometry analysis of non-oxidized PAPC (FIG. 13A), oxPAPC (FIG. 13B), PEIPC-enriched oxPAPC (FIG. 13C) and biotin-labeled oxPAPC (FIG. 13D).

FIGS. 14A-B. Oxidized phospholipids induce hyperactive cDC1 and cDC2 cells that display a hypermigratory phenotype. (A) Wild-type or NLRP3^{-/-} or Casp1/11^{-/-} BMDCs generated using FLT3L were either left untreated (none) or treated with LPS alone, or Alum alone, or PGPC alone for 24 h, or BMDCs were primed for 3 h with LPS, then treated with indicated stimuli for 21 h. IL-1β and TNFα release was monitored by ELISA. The percentage of cell death was measured by LDH release in the cell supernatants. Means and SDs from three replicates are shown and data are representative of at least three independent experiments. (B) Wild-type BMDCs generated using FLT3L were sorted as cDC1 or cDC2 cells then treated with indicated stimuli as in A. IL-1β and TNFα release was monitored by ELISA. The percentage of cell death was measured by LDH release in the cell supernatants. Means and SDs from three independent experiments done in two different laboratories.

FIGS. 15A-B. Hyperactive DCs induce strong CTL responses and a long lived anti-tumor immunity that is dependent on CCR7 expression and on inflammasome activation. (A-B) Wild-type BMDCs generated using FLT3L were either left untreated (DC^{naive}) or treated with LPS alone (DC^{active}) for 18 hours, or BMDCs were primed with LPS for 3 h then treated with PGPC (DC^{hyperactive}) or Alum (DC^{pyroptotic}) for 15 hours. Alternatively, BMDCs from NLRP3^{-/-} or

CCR7^{-/-} mice were primed with LPS for 3 hours then treated with PGPC for 15 hours. 1.10e6 BMDCs were incubated with OVA protein for 1 hour, then injected subcutaneously into wild-type mice. The injection of BMDCs that were not loaded with OVA protein served as a control group. 7 days post BMDCs injection, skin draining lymph nodes were dissected and stained with live-dead violet kit, OVA peptides tetramer antibodies, anti-CD45, anti-CD3, anti-CD8a, anti-CD4. (A) The percentage of SIINFEKL+ CD8+ T cells (upper panel), and AAHAEINEA+ CD4+ live T cells were measured by flow cytometry. (B) The absolute number of SIINFEKL+CD8+ T cells (upper panel) and AAHAEINEA+CD4+ live T cells were measured by flow cytometry using CounterBright beads.

FIGS. 16A-E. Hyperactive stimuli induce strong CTL response in an inflammasome dependent manner. (A) C57BL/6 mice were injected subcutaneously on the right flank with OVA either alone or with LPS or with PGPC, or with LPS plus oxPAPC or PGPC all emulsified in incomplete Freud's adjuvant (IFA). 7 or 40 days post immunization, T cells were isolated from the skin draining lymph nodes (dLN) by magnetic enrichment using anti-CD8 beads. (A) The percentage of T effector cells (Teff) as CD44^{low}CD62L^{low}, T effector memory cells (TEM) as CD444CD62L^{low}, and T central memory cells (TCM) as CD44^{hi}CD62L^{hi} are represented among CD3+CD8+ live cells. (B) CD8+ T cells were sorted from the dLN 7 days post immunization, then treated either with PMA plus ionomycin, or co-cultured with B160VA cells (target cells) at ratio of 1:3 (effector: target) for 5 h. The degranulation of CD8+ T cells was assessed by monitoring the percentage of CD107a+ among live CD8+ T cells using flow cytometry. Means and SDs of five-ten mice are shown. (C) Mice were injected s.c. on the right flank with OVA either alone or with LPS, or with LPS plus oxPAPC or PGPC all emulsified in IFA, or with LPS plus Alum. Alternatively, NLRP3^{-/-} mice were injected with OVA with LPS plus PGPC emulsified in IFA. 7 days post-immunization, CD8+ T cells were sorted from the skin dLN of immunized mice, and co-cultured with BMDCs loaded (or not) with OVA for 7 days at a ratio of 1:10 (DC: T cell). The percentage of SIINFEKL+ IFNγ+ among CD8+ live T cells was measured using OVA peptide tetramer staining followed by an intracellular IFNγ staining. (D-E) CD45.1 Mice were irradiated then reconstituted with Bone marrow ZBTB46DTR mice plus either WT or NLRP3^{-/-} or Casp1/11^{-/-} or CCR7^{-/-} (ratio 5:1), all on a CD45.2 C57BL/6 background. 6 weeks post-reconstitution, mouse chimeras were injected with tamoxifen every other day for 7 days. Chimeric mice were then immunized s.c. on the right flank with OVA with LPS plus PGPC emulsified in IFA. 7 days post-immunization, CD8+ T cells were isolated from the skin draining lymph nodes (dLN) or from the spleen by magnetic enrichment using anti-CD8 beads. (D) The percentage of Teff, TEM, TCM and T naïve cells in the skin dLN was measured by flow cytometry. (E) The percentage of SIINFEKL+ among CD8+ live T cells in the dLN (left panel) or in the spleen (right panel) was measured using OVA peptide tetramer staining by flow 40 cytometry. Total CD8+ T cells were sorted from the dLN and co-cultured with untreated BMDCs loaded (or not) with OVA for 7 days at a ratio of 1:10 (DC: T cell).

FIGS. 17A-D. The Immunization with hyperactivating stimuli eradicate tumors with immunogenicity ranging from hot to icy tumors. (A) C57BL/6 mice were inoculated subcutaneously with 5×10⁵ of live MC380VA cells on the left upper back. 14 days later, mice were either left untreated (unimmunized) or were injected subcutaneously on the right flank with syngeneic MC380VA whole tumor lysate (WTL), plus LPS and PGPC with or without injection of neutralizing anti IL-1β intravenously (i.v.), or anti-CD4, or anti-CD8a intraperitoneally. Mice received 2 boost injections with WTL and LPS plus PGPC on day 37 and on day 55 post tumor inoculation. Tumors were allowed to reach 20 mm of diameter. The percentage of survival is indicated (n=10 mice per group). (B) C57BL/6 mice were inoculated s.c. with 3×10⁵ live B16OVA cells on the left upper back. 10 days later, mice were either left untreated (unimmunized), or injected intraperitoneally with anti-PD1 antibody. Alternatively, mice were injected s.c. on the right flank with syngeneic B160VA WTL, plus LPS and PGPC with or without neutralizing antibodies anti IL-1β intravenously (i.v.), or anti-CD4, or anti-CD8a intraperitoneally. Mice received 2 boost injections with B160VA WTL, plus LPS and PGPC on day 17 and on day 24 post tumor inoculation. The percentage of survival is indicated (n=10 mice/group). (C) C57BL/6 mice were inoculated s.c. on the left upper back with 3×10⁵ live B16-F10 cells. 7 days later, mice were either left untreated (unimmunized), or injected intraperitoneally with anti-PD1 antibody. Alternatively, mice were immunized s.c. on the right flank with syngeneic B16-F10 WTL, plus LPS and PGPC with or without the neutralizing antibodies anti IL-1β intravenously (i.v.), or anti-CD4, or anti-CD8a intraperitoneally. Mice received 2 boost injections on day 14 and day 21 post tumor inoculation. The percentage of survival is indicated (n=10 mice per group). (D) BALB/c WT mice were inoculated s.c. with 3×10⁵ live CT26 cells on the left back. 7 days later, mice were either left untreated (unimmunized), or injected intraperitoneally with anti-PD1 antibody. Alternatively, mice were injected s.c. on the right flank with syngeneic CT26 WTL, plus LPS and PGPC with or without the neutralizing antibodies anti IL-1β intravenously (i.v.), or anti-CD4, or anti-CD8a intraperitoneally. Mice received 2 boost injections on day 14 and day 21 post tumor inoculation. The percentage of survival is indicated (n=10 mice/group).

FIG. 18A-F. Hyperactive cDC1s can use complex antigen sources to stimulate T cell mediated anti-tumor immunity. (A) Zbtb46DTR mice were s.c. injected with B160VA cells. Mice were either injected with diphteria toxin (DTx) every other day for 4 consecutive injections, or mice were injected with PBS. 7 days post tumor injection, all mice were immunized with B16OVA WTL plus LPS and PGPC, followed by two boosts injections. The percentage of mice survival is indicated (n=10 mice per group). (B) CD45.1 mice were irradiated then reconstituted with mixed BM from Zbtb46DTR mice plus either WT or Nlrp3^{-/-} or Casp1/11^{-/-} or Ccr7^{-/-} mice. 6 weeks post-reconstitution, mouse chimeras were injected s.c. with B61OVA cells, then all mice received DTx 3 times a week for a total of 12 consecutive injections. 7 days post tumor inoculation, chimeric mice were immunized with B160VA WTL and LPS plus PGPC and received two boosts injections. The percentage of mice survival is indicated (n=5 mice per group). (C-D) WT or Batf3^{-/-} mice were injected s.c with B16OVA cells. 7 days post-tumor inoculation, mice were either left untreated or WT and Batf3^{-/-} mice were immunized with B160VA WTL and LPS plus PGPC followed by two boosts injections. (C) The percentage of mice survival is indicated (n=10 mice per group). (D) 21 days post tumor inoculation, the percentage of OVA specific CD8+ T cells and CD4+ T cells were assessed using tetramer staining (n=5 mice per group). (E-F) Batf3^{-/-} mice were injected s.c on the right flank with B160VA cells. 7 days post tumor inoculation, mice were either left untreated (no cDC1 injection), or mice were injected s.c. on the left flank with FLT3-derived naïve cDC1s or active cDC1s treated with LPS or with hyperactive cDC1s pretreated with LPS plus PGPC. All cDC1s were loaded with B160VA WTL for 1 hour prior to their injection. (E) The percentage of mice survival is indicated (n=5 mice per group). (F) 21 days post tumor inoculation, OVA specific CD8+ T cells and CD4+ T cells were assessed using tetramer staining (n=5 mice per group).

FIGS. 19A-C. Oxidized phospholipids induce inflammasome dependent IL-1β secretion by cDC1 and cDC2 cells, and promote a hypermigratory DC phenotype. (A) Wild-type BMDCs generated using FLT3L were either left untreated (none) or treated with CpG 1806 alone, or PGPC alone for 24 h, or BMDCs were primed for 3 h with CpG 1806, then treated with indicated stimuli for 21 h. IL-1β and TNFα release was monitored by ELISA. The percentage of cell death was measured by LDH release in the cell supernatants. Means and SDs from three replicates are shown and data are representative of at least three independent experiments. (B) Gating strategy to isolate cDC1 or cDC2 from FLT3L-generated BMDCs or from the spleen of wild type mice. Purity post-sorting is indicated for splenic cDCs or FLT3L DCs. (C) splenic cDC1 or cDC2 were either left untreated (none) or treated with LPS alone, or Alum alone, or oxPAPC or PGPC alone for 18 h, or BMDCs were primed for 3 h with LPS, then treated with indicated stimuli for 15 h. IL-1β and TNFα release was monitored by ELISA. The percentage of cell death was measured by LDH release in the cell supernatants. Means and SDs from three independent experiments done in two different laboratories.

FIGS. 20A-C. Hyperactive DCs induce strong CTL responses and a long lived anti-tumor immunity that is dependent on CCR7 expression and on inflammasome activation. Wild-type BMDCs generated using FLT3L were either left untreated (DC^{naive}) or treated with LPS alone (DC^{active}) for 18 hours, or BMDCs were primed with LPS for 3 h then PGPC (DC^{hyperactive}) or Alum (DC^{pyroptotic}) were added to the culture media for 15 hours. Alternatively, BMDCs from NLRP3^{-/-} or CCR7^{-/-} mice were primed with LPS for 3 hours then PGPC was added to the culture media for 15 hours. BMDCs were washed then incubated with FITC labeled-OVA for 45 minutes, or with non-fluorescent OVA protein for 2 h. (A) OVA peptide presentation on MHC-I was monitored using PE-conjugated antibody to H-2Kb bound to the OVA peptide SIINFEKL. Data are represented as the frequency of SIINFEKL-associated DCs among CD11c+ live cells. Means and SDs from three replicates are shown and data are representative of three independent experiments. (B) Wild-type or NLRP3^{-/-} or CCR7^{-/-} BMDCs generated using FLT3L were stimulated as above. BMDCs were washed then stained with live-dead violet kit, CD11c and CD40. The mean fluorescence intensity (MFI) of surface CD40 (among CD11c+ live cells) was measured by flow cytometry. (C) CCR7^{-/-} BMDCs generated using FLT3L were either left untreated (none) or treated with LPS alone, or Alum alone, or PGPC alone for 24 h. Alternatively, BMDCs were primed for 3 h with LPS, then treated with indicated stimuli for 21 h. IL-1β and TNFα release was monitored by ELISA. The percentage of cell death was measured by LDH release in the cell supernatants. Means and SDs from three replicates are shown and data are representative of at least three independent experiments.

FIGS. 21A-D. Hyperactivating stimuli enhance memory T cell generation and potentiate antigen-specific IFNγ effector responses in an inflammasome-dependent manner. C57BL/6 mice were injected subcutaneously on the right flank with OVA either alone or with LPS or with PGPC, or with LPS plus oxPAPC or PGPC all emulsified in incomplete Freud's adjuvant (IFA). 7 days post immunization, T cells were isolated from the skin draining lymph nodes (dLN) by magnetic enrichment using anti-CD8 beads. (A) Gating strategy to determine the percentage of T effector cells (Teff) as CD44^{low}CD62L^{low}, T effector memory cells (TEM) as CD44^{high}CD62L^{low}, and T central memory cells (TCM) as CD44^{high}CD62L^{high}. (B) Absolute number of Teff or TEM cells in the skin dLN per mouse was assessed by flow cytometry among total CD3+ live cells. (C) CD8+ T cells were sorted from the dLN 7 days post immunization, then cultured with untreated BMDC loaded (or not) with a serial dilution of OVA protein starting from 1000 ug/ml. IFNγ cytokine secretion was measured by ELISA. Means and SDs of five mice are shown. (D) CD8+ T cells were sorted from the dLN 7 days post immunization, then treated either with PMA plus ionomycin, or co-cultured with B160VA cells (target cells) at ratio of 1:3 (effector: target) for 5 h. Gating strategy to determine the percentage of CD107a+ among live CD8+ T cells by flow cytometry. Each panel is representative of five mice. *P<0.05; **P<0.01.

FIGS. 22A-B. Hyperactivating stimuli enhance memory T cell generation and potentiate antigen-specific IFNγ effector responses in an inflammasome-dependent manner. (A-B) CD45.1 Mice were irradiated then reconstituted with Bone marrow ZBTB46DTR mice plus either WT or NLRP3^{-/-} or Casp1/11^{-/-} or CCR7^{-/-} (ratio 5:1), all on a CD45.2 C57BL/6 background. 6 weeks post-reconstitution, mouse chimeras were injected with tamoxifen every other day for 7 days. Chimeric mice were then immunized subcutaneously on the right flank with OVA with LPS plus PGPC emulsified in IFA. 7 days post-immunization, CD8+ T cells were isolated from the skin draining lymph nodes (dLN) or from the spleen by magnetic enrichment using anti-CD8 beads. (A) The percentage of Teff, TEM, TCM and T naïve cells in the skin dLN was measured by flow cytometry. Each panel is representative of five mice. (B) The percentage of SIINFEKL+ among CD8+ live T cells in the dLN (upper panel) or in the spleen (lower panel) was measured using OVA peptide tetramer staining by flow cytometry.

FIGS. 23A-C. (A-B) Mice were injected subcutaneously (s.c.) on the right flank with PBS (unimmunized), B16OVA cell lysate alone (none) or with LPS, or B16OVA lysate plus LPS and oxPAPC or PGPC all emulsified in incomplete Freud's adjuvant (IFA). 15 days post immunization, mice were challenged s.c. on the left upper back with 3×10⁵ of viable B160VA cells. 150 days later, tumor-free mice were re-challenged s.c. with 5×10⁵ of viable B16OVA cells on the back. (A) Tumor growth was monitored every 2 days (upper panel). For survival experiments (lower panel), mice were allowed to reach 20 mm of diameter (n=8-15 mice per group). (B-C) Tumors were harvested at the endpoint of tumor growth and dissociated to obtain single-cell tumor suspension. (B) The percentage of tumor infiltrating CD3+CD4+ and CD3+CD8+ T cells among enriched CD45+ live cells was assessed by flow cytometry. (C) Tumor infiltrating CD3+ T cells were sorted then stimulated for 24 h in the presence of anti-CD3 and anti-CD28 dynabeads. IFNγ release was measured by ELISA (lower panel) (n=4 mice per group).

FIGS. 24A-D. (A-B) Absolute number of CD8+ T cells and CD69+CD103+ T resident memory CD8+ T cells was assessed at the immunization or tumor injection site of survivor mice and measured by flow cytometry (n=4 mice). (C-D) Circulating memory CD8+ T cells (TCM) were isolated from the spleen, and T resident memory CD8+ cells (TRM) were isolated from the skin inguinal adipose tissue of survivor mice or age-matched unimmunized tumor-bearing mice. (C) TCM and TRM from survivor mice were co-cultured with B160VA or B16-F10 or CT26 tumor cells for 5 h at a ratio of 1:5 (tumor cell: T cell). Cell death by cytolytic CD8+ T cells was measured by LDH release in the supernatants. (D) Mice were either left untreated (no Tx), or were inoculated intravenously (i.v.) with 5×10⁵ of CD8+ TCM cells and/or intradermally (i.d.) with 5×10⁵ of CD8+ TRM cells isolated from survivor mice or age-matched unimmunized tumor-bearing mice. 7 days later, all mice were challenged with 3×10⁵ of viable B160VA cells. Percentage of survival was monitored every 2 days. Mice were allowed to reach 20 mm of diameter (n=5 mice per group).

### DETAILED DESCRIPTION

The present disclosure is based, in part, on the finding that stimuli which activate dendritic cells (DCs), or promote DC pyroptosis, induce a mixed T cell response consisting of type I and type 2 T helper (Th) cells. Stimuli that hyperactivate DCs, in contrast, selectively stimulate TH1 and cytotoxic T lymphocyte (CTL) immune responses, with no evidence of TH2-induced immunity. The TH1-biased immunity generated by hyperactive DCs endows these cells with the unique ability to mediate long-term protective anti-tumor immunity, even when a complex antigen source is used (e.g. tumor cell lysates). Stimuli that promote a traditional DC activation state or pyroptosis have no ability to adjuvant tumor cell lysates and offer minimal protection against tumors. These novel attributes are intrinsic to hyperactive DCs and depend on IL-1β and inflammasome components. The resulting tumor-specific T cells can be transferred to recipient mice and confer complete protection from subsequent challenges. Hyperactivating stimuli induce protective immunity to tumors that are sensitive or resistant to PD-1 checkpoint blockade. These collective findings establish the physiological importance of the hyperactive state of DCs and open avenues for novel strategies of cancer immunotherapies that are agnostic to the identity of tumor antigens.

Thus, provided herein are methods for producing or enhancing an adaptive immune response in a subject and for treating cancer in a subject. The methods are useful, e.g., for therapeutic and/or prophylactic cancer vaccination.

Provided herein are methods of inducing or enhancing an adaptive immune response to a cancer in a subject that comprise administering an effective amount of (i) a Toll-Like Receptor (TLR) ligand; (ii) a non-canonical inflammasome-activating lipid; and (iii) an cancer immunogen to the subject.

Also provided herein are methods of treating cancer in a subject that comprise administering an effective amount of (i) a Toll-Like Receptor (TLR) a TLR ligand; (ii) a non-canonical inflammasome-activating lipid, and (iii) a cancern immunogen to the subject.

Preferably, the methods described herein inhibit the growth or progression of cancer, e.g., a tumor, or a viral infection in a subject. For example, the methods described herein inhibit the growth of a tumor by at least 1%, e.g., by at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, at least 80%, at least 90% or 100%. In other cases, the methods described herein reduce the size of a tumor by at least 1 mm in diameter, e.g., by at least 2 mm in diameter, by at least 3 mm in diameter, by at least 4 mm in diameter, by at least 5 mm in diameter, by at least 6 mm in diameter, by at least 7 mm in diameter, by at least 8 mm in diameter, by at least 9 mm in diameter, by at least 10 mm in diameter, by at least 11 mm in diameter, by at least 12 mm in diameter, by a least 13 mm in diameter, by at least 14 mm in diameter, by at least 15 mm in diameter, by at least 20 mm in diameter, by at least 25 mm in diameter, by at least 30 mm in diameter, by at least 40 mm in diameter, by at least 50 mm in diameter or more. In some cases, the subject has had the bulk of the tumor resected.

Other aspects are described infra.

### Definitions

The articles “a” and “an” are used herein to refer to one or to more than one (i.e., to at least one) of the grammatical object of the article. By way of example, “an element” means one element or more than one element. Thus, recitation of “a cell”, for example, includes a plurality of the cells of the same type. Furthermore, to the extent that the terms “including”, “includes”, “having”, “has”, “with”, or variants thereof are used in either the detailed description and/or the claims, such terms are intended to be inclusive in a manner similar to the term “comprising.” “About” as used herein when referring to a measurable value such as an amount, a temporal duration, and the like, is meant to encompass variations of +/−20%, +/−10%, +/−5%, +/−1%, or +/−0.1% from the specified value, as such variations are appropriate to perform the disclosed methods. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

By “cancer” as used herein is meant, a disease, condition, trait, genotype or phenotype characterized by unregulated cell growth or replication as is known in the art; including colorectal cancer, as well as, for example, leukemias, e.g., acute myelogenous leukemia (AML), chronic myelogenous leukemia (CML), acute lymphocytic leukemia (ALL), and chronic lymphocytic leukemia, AIDS related cancers such as Kaposi's sarcoma; breast cancers; bone cancers such as Osteosarcoma, Chondrosarcomas, Ewing's sarcoma, Fibrosarcomas, Giant cell tumors, Adamantinomas, and Chordomas; Brain cancers such as Meningiomas, Glioblastomas, Lower-Grade Astrocytomas, Oligodendrocytomas, Pituitary Tumors, Schwannomas, and Metastatic brain cancers; cancers of the head and neck including various lymphomas such as mantle cell lymphoma, non-Hodgkins lymphoma, adenoma, squamous cell carcinoma, laryngeal carcinoma, gallbladder and bile duct cancers, cancers of the retina such as retinoblastoma, cancers of the esophagus, gastric cancers, multiple myeloma, ovarian cancer, uterine cancer, thyroid cancer, testicular cancer, endometrial cancer, melanoma, lung cancer, bladder cancer, prostate cancer, lung cancer (including non-small cell lung carcinoma), pancreatic cancer, sarcomas, Wilms' tumor, cervical cancer, head and neck cancer, skin cancers, nasopharyngeal carcinoma, liposarcoma, epithelial carcinoma, renal cell carcinoma, gallbladder adeno carcinoma, parotid adenocarcinoma, endometrial sarcoma, multidrug resistant cancers; and proliferative diseases and conditions, such as neovascularization associated with tumor angiogenesis, macular degeneration (e.g., wet/dry AMD), corneal neovascularization, diabetic retinopathy, neovascular glaucoma, myopic degeneration and other proliferative diseases and conditions.

As used herein, the term “pattern recognition receptor ligand” refers to molecular compounds that activate one or more members of the Toll-like Receptor (TLR) family, RIG-I like Receptor (RLR) family, Nucleotide binding leucine rich repeat containing (NLR) family, cGAS, STING or AIM2-like Receptors (ALRs). Specific examples of pattern recognition receptor ligands include natural or synthetic bacterial lipopolysaccharides (LPS), natural or synthetic bacterial lipoproteins, natural or synthetic DNA or RNA sequences, natural or synthetic cyclic dinucleotides, and natural or synthetic carbohydrates. Cyclic dinucleotides include cyclic GMP-AMP (cGAMP), cyclic di-AMP, cyclic di-GMP.

As used herein, the term “cancer therapy” refers to a therapy useful in treating cancer. Examples of anti-cancer therapeutic agents include, but are not limited to, e.g., surgery, chemotherapeutic agents, immunotherapy, growth inhibitory agents, cytotoxic agents, agents used in radiation therapy, anti-angiogenesis agents, apoptotic agents, anti-tubulin agents, and other agents to treat cancer, such as anti-HER-2 antibodies (e.g., HERCEPTIN™), anti-CD20 antibodies, an epidermal growth factor receptor (EGFR) antagonist (e.g., a tyrosine kinase inhibitor), HER1/EGFR inhibitor (e.g., erlotinib (TARCEVA™)), platelet derived growth factor inhibitors (e.g., GLEEVECT™ (Imatinib Mesylate)), a COX-2 inhibitor (e.g., celecoxib), interferons, cytokines, antagonists (e.g., neutralizing antibodies) that bind to one or more of the following targets ErbB2, ErbB3, ErbB4, PDGFR-beta, BlyS, APRIL, BCMA or VEGF receptor(s), TRAIL/Apo2, and other bioactive and organic chemical agents, etc. Combinations thereof are also contemplated for use with the methods described herein.

A “chemotherapeutic agent” is a chemical compound useful in the treatment of cancer. Examples of chemotherapeutic agents include Erlotinib (TARCEVA™, Genentech/OSI Pharm.), Bortezomib (VELCADE™, Millennium Pharm.), Fulvestrant (FASLODEX™, Astrazeneca), Sutent (SUl1248, Pfizer), Letrozole (FEMARA™, Novartis), Imatinib mesylate (GLEEVEC™, Novartis), PTK787/ZK 222584 (Novartis), Oxaliplatin (Eloxatin™, Sanofi), 5-FU (5-fluorouracil), Leucovorin, Rapamycin (Sirolimus, RAPAMUNE™, Wyeth), Lapatinib (GSK572016, GlaxoSmithKline), Lonafarnib (SCH 66336), Sorafenib (BAY43-9006, Bayer Labs.), and Gefitinib (IRESSA™, Astrazeneca), AG1478, AG1571 (SU 5271; Sugen), alkylating agents such as Thiotepa and CYTOXAN™ cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analogue topotecan); bryostatin; callystatin; CC-1065 (including its adozcicsin, carzcicsin and bizcicsin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin γ1 and calicheamicin omega 1 (Angew Chem. Intl. Ed. Engl. (1994) 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, anthramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN™ doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, strcptonigrin, strcptozocin, tubcrcidin, ubenimcx, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacytidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK™ polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosinc; arabinoside (“Ara-C”); cyclophosphamidc; thiotcpa; taxoids, e.g., TAXOL™ paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE™ doxetaxel (Rhone-Poulenc Rorer, Antony, France); chloranbucil; GEMZAR™ gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE™ vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above.

Also included in this definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX™ (tamoxifen)), raloxifene, droloxifene, 4-hydroxytamoxifen, trioxifene, keoxifene, LY117018, onapristone, and FARESTON™ (toremifene); (ii) aromatase inhibitors that inhibit the enzyme aromatase, which regulates estrogen production in the adrenal glands, such as, for example, 4(5)-imidazoles, aminoglutethimide, MEGASE™ (megestrol acetate), AROMASIN™ (exemestane), formestanie, fadrozole, RIVISOR™ (vorozole), FEMARA™ (letrozole), and ARIMIDEX™ (anastrozole); (iii) anti-androgens such as flutamide, nilutamide, bicalutamide, leuprolide, and goserelin; as well as troxacitabine (a 1,3-dioxolane nucleoside cytosine analog); (iv) aromatase inhibitors; (v) protein kinase inhibitors; (vi) lipid kinase inhibitors; (vii) antisense oligonucleotides, particularly those which inhibit expression of genes in signaling pathways implicated in aberrant cell proliferation, such as, for example, PKC-alpha, Ralf and H-Ras; (viii) ribozymes such as a VEGF expression inhibitor (e.g., ANGIOZYME™ (ribozyme)) and a HER2 expression inhibitor; (ix) vaccines such as gene therapy vaccines, for example, ALLOVECTIN™ vaccine, LEUVECTIN™ vaccine, and VAXID™ vaccine; PROLEUKIN™ rIL-2; LURTOTECAN™ topoisomerase 1 inhibitor; ABARELIX™ rmRH; (x) anti-angiogenic agents such as bevacizumab (AVASTIN™, Genentech); and (xi) pharmaceutically acceptable salts, acids or derivatives of any of the above.

The term “checkpoint inhibitor” means a group of molecules on the cell surface of CD4⁺ and/or CD8⁺ T cells that fine-tune immune responses by down-modulating or inhibiting an anti-tumor immune response. Immune checkpoint proteins are well known in the art and include, without limitation, CTLA-4, PD-1, VISTA, B7-H2, B7-H3, PD-L1, B7-H4, B7-H6, 2B4, ICOS, HVEM, PD-L2, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, and A2aR (see, for example, WO 2012/177624). “Anti-immune checkpoint inhibitor therapy” refers to the use of agents that inhibit immune checkpoint inhibitors. Inhibition of one or more immune checkpoint inhibitors can block or otherwise neutralize inhibitory signaling to thereby upregulate an immune response in order to more efficaciously treat cancer. Exemplary agents useful for inhibiting immune checkpoint inhibitors include antibodies, small molecules, peptides, peptidomimetics, natural ligands, and derivatives of natural ligands, that can either bind and/or inactivate or inhibit immune checkpoint proteins, or fragments thereof; as well as RNA interference, antisense, nucleic acid aptamers, etc. that can downregulate the expression and/or activity of immune checkpoint inhibitor nucleic acids, or fragments thereof. Exemplary agents for upregulating an immune response include antibodies against one or more immune checkpoint inhibitor proteins block the interaction between the proteins and its natural receptor(s); a non-activating form of one or more immune checkpoint inhibitor proteins (e.g., a dominant negative polypeptide); small molecules or peptides that block the interaction between one or more immune checkpoint inhibitor proteins and its natural receptor(s); fusion proteins (e.g. the extracellular portion of an immune checkpoint inhibition protein fused to the Fe portion of an antibody or immunoglobulin) that bind to its natural receptor(s); nucleic acid molecules that block immune checkpoint inhibitor nucleic acid transcription or translation; and the like. Such agents can directly block the interaction between the one or more immune checkpoint inhibitors and its natural receptor(s) (e.g., antibodies) to prevent inhibitory signaling and upregulate an immune response. Alternatively, agents can indirectly block the interaction between one or more immune checkpoint proteins and its natural receptor(s) to prevent inhibitory signaling and upregulate an immune response. For example, a soluble version of an immune checkpoint protein ligand such as a stabilized extracellular domain can binding to its receptor to indirectly reduce the effective concentration of the receptor to bind to an appropriate ligand. In one embodiment, anti-PD-1 antibodies, anti-PD-L1 antibodies, and anti-CTLA-4 antibodies, either alone or used in combination.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

“Concurrently administered” as used herein means that two compounds are administered sufficiently close in time to achieve a combined immunological effect. Concurrent administration can thus be carried out by sequential administration or simultaneous administration (e.g., simultaneous administration in a common, or the same, carrier).

An “effective amount” as used herein, means an amount which provides a therapeutic or prophylactic benefit.

“Immunogen” and “antigen” are used interchangeably and mean any compound to which a cellular or humoral immune response is to be directed against. Non-living immunogens include, e.g., tumor cell lysates, tumor proteins, tumor lipids, tumor carbohydrates, killed immunogens, subunit vaccines, recombinant proteins or peptides or the like. The adjuvants described herein can be used with any suitable immunogen. Exemplary immunogens of interest include those constituting or derived from a virus, a mycoplasma, a bacterium, a parasite, a protozoan, a prion or the like. Accordingly, an immunogen of interest can be from, without limitation, a human papilloma virus, a herpes virus such as herpes simplex or herpes zoster, a retrovirus such as human immunodeficiency virus 1 or 2, a hepatitis virus, an influenza virus, a rhinovirus, respiratory syncytial virus, cytomegalovirus, adenovirus, Mycoplasma pneumoniae, a bacterium of the genus Salmonella, Staphylococcus, Streptococcus, Enterococcus, Clostridium, Escherichia, Klebsiella, Vibrio, Mycobacterium, amoeba, a malarial parasite, and/or Trypanosoma cruzi.

As used herein, the term “in combination” in the context of the administration of a therapy to a subject refers to the use of more than one therapy for therapeutic benefit. The term “in combination” in the context of the administration can also refer to the prophylactic use of a therapy to a subject when used with at least one additional therapy. The use of the term “in combination” does not restrict the order in which the therapies (e.g., a first and second therapy) are administered to a subject. A therapy can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject which had, has, or is susceptible to cancer. The therapies are administered to a subject in a sequence and within a time interval such that the therapies can act together. In a particular embodiment, the therapies are administered to a subject in a sequence and within a time interval such that they provide an increased benefit than if they were administered otherwise. Any additional therapy can be administered in any order with the other additional therapy.

The “modulation” of, e.g., a symptom, level or biological activity of a molecule, or the like, refers, for example, to the symptom or activity, or the like that is detectably increased or decreased. Such increase or decrease can be observed in treated subjects as compared to subjects not treated with an adjuvant lipid as described herein (a non-canonical inflammasome-activating lipid), where the untreated subjects (e.g., subjects administered immunogen in the absence of adjuvant lipid) have, or are subject to developing, the same or similar disease or infection as treated subjects. Such increases or decreases can be at least about 2%, 5%, 10%, 15%, 20%, 25%, 30%, 40%, 50%, 60%, 70%, 75%, 80%, 85%, 90%, 95%, 98%, 100%, 150%, 200%, 250%, 300%, 400%, 500%, 1000% or more or within any range between any two of these values. Modulation can be determined subjectively or objectively, e.g., by the subject's self-assessment, by a clinician's assessment or by conducting an appropriate assay or measurement, including, e.g., assessment of the extent and/or quality of immunostimulation in a subject achieved by an administered immunogen in the presence of an adjuvant lipid as described herein (a non-canonical inflammasome-activating lipid). Modulation can be transient, prolonged or permanent or it can be variable at relevant times during or after an adjuvant lipid as described herein is administered to a subject or is used in an assay or other method described herein or a cited reference, e.g., within times described infra, or about 12 hours to 24 or 48 hours after the administration or use of an adjuvant lipid as described herein to about 3, 4, 5, 6, 7, 8, 9, 10, 11, 12, 13, 14, 21, 28 days, or 1, 3, 6, 9 months or more after a subject(s) has received such an immunostimulatory composition/treatment.

The term “non-canonical inflammasome-activating lipid”, as used herein, refers to a lipid capable of eliciting the hyperactivating of a dendritic cell or macrophage. Exemplary “non-canonical inflammasome-activating lipids” include PAPC, oxPAPC and species of oxPAPC (e.g., HOdiA-PC, KOdiA-PC, HOOA-PC, KOOA-PC, POVPC, PGPC).

Species of oxPAPC are known and described in the art. See. e.g., Ni et al., “Evaluation of Air Oxidized PAPC: A Multi Laboratory Study by LC-MS/MS,” Free Radical Biology and Medicine 144:156-66 (2019); Table 1.

In some embodiments, the non-canonical inflammasome-activating lipid comprises 2-[[(2R)-2-[(E)-7-carboxy-5-hydroxyhept-6-enoyl]oxy-3-hexadecanoyloxypropoxy]-hydroxyphosphoryl]oxyethyl-trimethylazanium (HOdiA-PC), [(2R)-2-[(E)-7-carboxy-5-oxohept-6-enoyl]oxy-3-hexadecanoyloxypropyl] 2-(trimethylazaniumyl)ethyl phosphate (KOdiA-PC), 1-palmitoyl-2-(5-hydroxy-8-oxo-octenoyl)-sn-glycero-3-phosphorylcholine (HOOA-PC), 2-[[(2R)-2-[(E)-5,8-dioxooct-6-enoyl]oxy-3-hexadecanoyloxypropoxy]-hydroxyphosphoryl]oxyethyl-trimethylazanium (KOOA-PC), [(2R)-3-hexadecanoyloxy-2-(5-oxopentanoyloxy)propyl] 2-(trimethylazaniumyl)ethyl phosphate (POVPC), [(2R)-2-(4-carboxybutanoyloxy)-3-hexadecanoyloxypropyl] 2-(trimethylazaniumyl)ethyl phosphate (PGPC), [(2R)-3-hexadecanoyloxy-2-[4-[3-[(E)-[2-[(Z)-oct-2-enyl]-5-oxocyclopent-3-en-1-ylidene]methyl]oxiran-2-yl]butanoyloxy]propyl] 2-(trimethylazaniumyl)ethyl phosphate (PECPC), [(2R)-3-hexadecanoyloxy-2-[4-[3-[(E)-[3-hydroxy-2-[(Z)-oct-2-enyl]-5-oxocyclopentylidene]methyl]oxiran-2-yl]butanoyloxy]propyl] 2-(trimethylazaniumyl)ethyl phosphate (PEIPC), or a combination thereof.

In some embodiments, the oxPAPC species is an oxPAPC species set forth in Table 1, or combinations thereof.

TABLE 1
Oxidized PAPC molecular species identified in Ni et al., with corresponding
elemental composition (neutral), exact mass, adduct, m/z, ID and proposed structures.

| Elemental | | | | | | | | | |
|---|---|---|---|---|---|---|---|---|---|
| composition | Exact | | | | | | | | |
| (neutral) | mass | Adduct | m/z | ID | L1 | L2 | L3 | L4 | Proposed structures |
| C24H50NO7P | 495.3325 | [M + HCOO]− | 540.3301 | PC(16:0/0:0) | x | | x | x | PC(16:0/0:0) |
| C28H50NO7P | 543.3325 | [M + HCOO]− | 588.3301 | PC(0:0/20:4) | | | | x | PC(0:0/20:4) |
| C28H54NO10P | 595.3485 | [M − H]− | 594.3407 | PC(16:0/4:0 | | | x | | PC(16:0/4:0<COOH@C4>) |
| | | | | <COOH@C4>) | | | | | |
| C29H56NO10P | 609.3642 | [M − H]− | 608.3564 | PC(16:0/5:0 | x | | x | x | PC(16:0/5:0<COOH@C5>) |
| | | | | <COOH@C5>) | | | | | |
| C28H54NO9P | 579.3536 | [M + HCOO]− | 624.3513 | PC(16:0/4:0 | x | | x | x | PC(16:0/4:0<CHO@C4>) |
| | | | | <CHO@C4>) | | | | | |
| C31H58NO10P | 635.3798 | [M − H]− | 634.3720 | PC(16:0/7:1[1 × DB] | | | x | x | PC(16:0/7:1[1 × DB]<COOH@C7>) |
| | | | | <COOH@C7>) | | | | | |
| C29H56NO9P | 593.3693 | [M + HCOO]− | 638.3669 | PC(16:0/5:0 | x | | x | x | PC(16:0/5:0<CHO@C5>) |
| | | | | <CHO@C5>) | | | | | |
| C32H60NO10P | 649.3955 | [M − H]− | 648.3877 | PC(16:0/8:1[1 × DB] | | | x | x | PC(16:0/8:1[1 × DB]<COOH@C8>) |
| | | | | <COOH@C8>) | | | | | |
| C31H58NO11P | 651.3748 | [M − H]− | 650.3669 | PC(16:0/7:1[1 × DB] | | | x | | PC(16:0/7:1[1 × DB, 1 × OH]COOH@C7>) |
| | | | | <COOH@C7>) + O | | | | | |
| C32H58NO11P | 663.3748 | [M − H]− | 662.3669 | PC(16:0/8:1[1 × DB] | x | | x | x | PC(16:0/8:1[1 × DB, 1 × KETO]<COOH@C8>) |
| | | | | <COOH@C8>) + | | | | | |
| | | | | O—2H | | | | | |
| C31H58NO9P | 619.3849 | [M + HCOO]− | 664.3826 | PC(16:0/7:1[1 × DB] | x | x | x | x | PC(16:0/7:1[1 × DB]<CHO@C7>) |
| | | | | <CHO@C7>) | | | | | |
| C31H56NO10P | 633.3642 | [M + HCOO]− | 678.3618 | PC(16:0/7:1[1 × DB] | | | x | | PC(16:0/7:1[1 × DB, 1 × KETO]<CHO@C7>) |
| | | | | <CHO@C7>) + | | | | | |
| | | | | O—2H | | | | | |
| C31H58NO10P | 635.3798 | [M + HCOO]− | 680.3775 | PC(16:0/7:1[1 × DB] | | | x | | PC(16:0/7:1[1 × DB, 1 × OH]<CHO@C7>) |
| | | | | <CHO@C7>) + O | | | | | |
| C32H62NO12P | 683.4010 | [M − H]− | 682.3931 | PC(16:0/8:0 | | | x | | PC(16:0/8:0[2 × OH]<COOH@C8>) |
| | | | | <COOH@C8>) + 2O | | | | | |
| C34H60NO11P | 689.3904 | [M − H]− | 688.3826 | PC(16:0/10:2[2 × DB] | | | x | x | PC(16:0/10:2[2 × DB, |
| | | | | <COOH@C10>) + | | | | | 1 × KETO]<COOH@C10>) |
| | | | | O—2H | | | | | |
| C35H64NO10P | 689.4268 | [M − H]− | 688.4190 | PC(16:0/11:2[2 × DB] | | | x | | PC(16:0/11:2[2 × DB]<COOH@C11>) |
| | | | | <COOH@C11>) | | | | | |
| C32H58NO10P | 647.3798 | [M + HCOO]− | 692.3775 | PC(16:0/8:1[1 × DB] | x | | | x | PC(16:0/8:1[1 × DB, 1 × KETO]<CHO@C8>) |
| | | | | <CHO@C8>) + | | | | | |
| | | | | O—2H | | | | | |
| C32H60NO10P | 649.3955 | [M + HCOO]− | 694.3931 | PC(16:0/8:1[1 × DB] | x | | x | x | PC(16:0/8:1[1 × DB, 1 × OH]<CHO@C8>) |
| | | | | <CHO@C8>) + O | | | | | PC(16:0/8:0[1 × EPOXY]<CHP@C8>) |
| C31H60NO11P | 653.3904 | [M + HCOO]− | 698.3881 | PC(16:0/7:1[1 × DB] | x | | x | x | PC(16:0/7:0[2 × OH]<CHO@C7>) |
| | | | | <CHO@C7>) + 2O | | | | | PC(16:0/7:0[1 × OOH]<CHO@C7>) |
| C34H62NO9P | 659.4162 | [M + HCOO]− | 704.4139 | PC(16:0/10:2[2 × DB] | x | x | x | x | PC(16:0/10:2[2 × DB]<CHO@C10>) |
| | | | | <CHO@C10>) | | | | | |
| C32H60NO11P | 665.3904 | [M + HCOO]− | 710.3881 | PC(16:0/8:1[1 × DB] | x | | x | x | PC(16:0/8:1[1 × DB, 1 × OOH]<CHO@C8>) |
| | | | | <CHO@C8>) + 2O | | | | | PC(16:0/8:1[1 × DB, 2 × OH]<CHO@C8>) |
| | | | | | | | | | PC(16:0/8:0[1 × OH, 1 × EPOXY]<CHO@C8>) |
| C32H62NO11P | 667.4061 | [M + HCOO]− | 712.4037 | PC(16:0/8:0 | x | | x | x | PC(16:0/8:0[2 × OH]<CHO@C8>) |
| | | | | <CHO@C8>) + 2O | | | | | PC(16:0/8:0[1 × OOH]<CHO@C8>) |
| C35H62NO12P | 719.4010 | [M − H]− | 718.3931 | PC(16:0/11:2[2 × DB] | | | x | x | PC(16:0/11:3[3 × DB, 2 × OH]<COOH@C11>) |
| | | | | <COOH@C11>) + | | | | | PC(16:0/11:2[2 × DB, 1 × OH, 1 × KETO] |
| | | | | 2O—2H | | | | | <COOH@C11>) |
| C35H64NO9P | 673.4319 | [M + HCOO]− | 718.4295 | PC(16:0/11:2[2 × DB] | | | x | x | PC(16:0/11:2[2 × DB]<CHO@C11>) |
| | | | | <CHO@C11>) | | | | | |
| C34H62NO10P | 675.4111 | [M + HCOO]− | 720.4088 | PC(16:0/10:2[2 × DB] | | | x | x | PC(16:0/10:2[2 × DB, 1 × OH]<CHO@C10>) |
| | | | | <CHO@C10>) + O | | | | | |
| C34H58NO11P | 687.3748 | [M + HCOO]− | 732.3724 | PC(16:0/10:2[2 × DB] | | | x | x | PC(16:0/10:2[2 × DB, 2 × KETO]<CHO@C10>) |
| | | | | <CHO@C10>) + | | | | | |
| | | | | 2O—4H | | | | | |
| C35H62NO10P | 687.4111 | [M + HCOO]− | 732.4088 | PC(16:0/11:2[2 × DB] | | | x | x | PC(16:0/11:2[2 × DB, 1 × KETO]<CHO@C11>) |
| | | | | <CHO@C11>) + | | | | | |
| | | | | O—2H | | | | | |
| C35H64NO10P | 689.4268 | [M + HCOO]− | 734.4244 | PC(16:0/11:2[2 × DB] | x | | | x | PC(16:0/11:2[2 × DB, 1 × OH]<CHO@C11>) |
| | | | | <CHO@C11>) + O | | | | | PC(16:0/11:1[1 × DB, 1 × EPOXY]<CHO@C11>) |
| C34H62NO11P | 691.4061 | [M + HCOO]− | 736.4037 | PC(16:0/10:2[2 × DB] | | | x | x | PC(16:0/10:2[2 × DB, 1 × OOH]<CHO@C10>) |
| | | | | <CHO@C10>) + 2O | | | | | PC(16:0/10:2[2 × DB, 2 × OH]<CHO@C10>) |
| | | | | | | | | | PC(16:0/10:1[1 × DB, 1 × OH, 1 × EPOXY] |
| | | | | | | | | | <CHO@C10>) |
| | | | | | | | | | PC(16:0/10:0[2 × EPOXY]<CHO@C10>) |
| C37H66NO9P | 699.4475 | [M + HCOO]− | 744.4452 | PC(16:0/13:3[3 × DB] | x | | x | x | PC(16:0/13:3[3 × DB]<CHO@C13>) |
| | | | | <CHO@C13>) | | | | | |
| C35H62NO11P | 703.4061 | [M + HCOO]− | 748.4037 | PC(16:0/11:2[2 × DB] | | | | x | PC(16:0/11:2[2 × DB, 1 × OH, 1 × KETO] |
| | | | | <CHO@C11>) + | | | | | <CHO@C11>) |
| | | | | 2O—2H | | | | | |
| C35H64NO11P | 705.4217 | [M + HCOO]− | 750.4194 | PC(16:0/11:2[2 × DB] | | | x | x | PC(16:0/11:2[2 × DB, 1 × OOH]<CHO@C11>) |
| | | | | <CHO@C11>) + 2O | | | | | PC(16:0/11:2[2 × DB, 2 × OH]<CHO@C11>) |
| | | | | | | | | | PC(16:0/11:1[1 × DB, 1 × OH, 1 × EPOXY] |
| | | | | | | | | | <CHO@C11>) |
| | | | | | | | | | PC(16:0/11:0[2 × EPOXY]<CHO@C11>) |
| C37H64NO10P | 713.4268 | [M + HCOO]− | 758.4244 | PC(16:0/13:3[3 × DB] | | | x | x | PC(16:0/13:3[3 × DB, 1 × KETO]<CHO@C13>) |
| | | | | <CHO@C13>) + | | | | | |
| | | | | O—2H | | | | | |
| C37H66NO10P | 715.4424 | [M + HCOO]− | 760.4401 | PC(16:0/13:3[3 × DB] | | | | x | PC(16:0/13:3[3 × DB, 1 × OH]<CHO@C13>) |
| | | | | <CHO@C13>) + O | | | | | |
| C35H62NO12P | 719.4010 | [M + HCOO]− | 764.3986 | PC(16:0/11:2[2 × DB] | | | | x | PC(16:0/11:2[2 × DB, 1 × KETO, 1 × OOH] |
| | | | | <CHO@C11>) + | | | | | <CHO@C11>) |
| | | | | 3O—2H | | | | | PC(16:0/11:2[2 × DB, 2 × OH, 1 × |
| | | | | | | | | | KETO]<CHO@C11>) |
| C35H64NO12P | 721.4166 | [M + HCOO]− | 766.4143 | PC(16:0/11:2[2 × DB] | | | x | x | PC(16:0/11:2[2 × DB, 1 × OH, 1 × |
| | | | | <CHO@C11>) + 3O | | | | | OOH]<CHO@C11>) |
| | | | | | | | | | PC(16:0/11:2[2 × DB, 3 × OH]<CHO@C11>) |
| | | | | | | | | | PC(16:0/11:1[1 × DB, 2 × OH, 1 × |
| | | | | | | | | | EPOXY]<CHO@C11>) |
| | | | | | | | | | PC(16:0/11:0[1 × OH, 2 × EPOXY]<CHO@C11>) |
| C35H66NO12P | 723.4323 | [M + HCOO]− | 768.4299 | PC(16:0/11:1[1 × DB] | | | x | x | PC(16:0/11:1[1 × DB, 3 × OH]<CHO@C11>) |
| | | | | <CHO@C11>) + 3O | | | | | PC(16:0/11:1[1 × DB, 1 × OH, 1 × |
| | | | | | | | | | OOH]<CHO@C11>) |
| | | | | | | | | | PC(16:0/11:0[1 × OOH, 1 × |
| | | | | | | | | | EPOXY]<CHO@C11>) |
| C37H66NO11P | 731.4374 | [M + HCOO]− | 776.4350 | PC(16:0/13:3[3 × DB] | x | | x | x | PC(16:0/13:3[3 × DB, 1 × OOH]<CHO@C13>) |
| | | | | <CHO@C13>) + 2O | | | | | PC(16:0/13:3[3 × DB, 2 × OH]<CHO@C13>) |
| | | | | | | | | | PC(16:0/13:2[2 × DB, 1 × OH, 1 × |
| | | | | | | | | | EPOXY]<CHO@C13>) |
| | | | | | | | | | PC(16:0/13:1[1 × DB, 2 × EPOXY] |
| | | | | | | | | | <CHO@C13>) |
| C35H64NO13P | 737.4115 | [M + HCOO]− | 782.4092 | PC(16:0/11:2[2 × DB] | | | x | x | PC(16:0/11:2[2 × DB, 2 × OOH]<CHO@C11>) |
| | | | | <CHO@C11>) + 4O | | | | | PC(16:0/11:2[2 × DB, 2 × OH, 1 × |
| | | | | | | | | | OOH]<CHO@C11>) |
| | | | | | | | | | PC(16:0/11:1[1 × DB,1 × OH, 1 × OOH, 1 × |
| | | | | | | | | | EPOXY]<CHO@C11>) |
| | | | | | | | | | PC(16:0/11:0[1 × OOH, 2 × |
| | | | | | | | | | EPOXY]<CHO@C11>) |
| C35H66NO13P | 739.4272 | [M + HCOO]− | 784.4248 | PC(16:0/11:1[1 × DB] | | | x | x | PC(16:0/11:1[1 × DB, 2 × OOH]<CHO@C11>) |
| | | | | <CHO@C11>) + 4O | | | | | |
| C44H78NO9P | 795.5414 | [M + HCOO]− | 840.5391 | PC(16:0/20:4 | x | x | x | x | PC(16:0/20:4[4 × DB, 1× KETO]) |
| | | | | [4 × DB]) + O—2H | | | | | |
| C44H80NO9P | 797.5571 | [M + HCOO]− | 842.5547 | PC(16:0/20:4 | x | x | x | x | PC(16:0/20:4[4 × DB, 1 × OH]) |
| | | | | [4 × DB]) + O | | | | | PC(16:0/20:3[3 × DB, 1 × EPOXY]) |
| C44H76NO10P | 809.5207 | [M + HCOO]− | 854.5183 | PC(16:0/20:4 | x | | x | x | PC(16:0/20:4[4 × DB, 2 × KETO]) |
| | | | | [4 × DB]) + 2O—4H | | | | | PC(16:0/[epoxyisoprostane D2 —H2O]) |
| | | | | | | | | | PC(16:0/[epoxyisoprostane E2 —H2O]) |
| C44H80NO10P | 813.5520 | [M + HCOO]− | 858.5496 | PC(16:0/20:4 | x | x | x | x | PC(16:0/20:4[4 × DB, 1 × OOH]) |
| | | | | [4 × DB]) + 2O | | | | | PC(16:0/20:4[4 × DB, 2 × OH]) |
| | | | | | | | | | PC(16:0/20:3[3 × DB, 1× OH, 1 × EPOXY]) |
| | | | | | | | | | PC(16:0/20:2[2 × DB, 2 × EPOXY]) |
| C44H78NO11P | 827.5313 | [M + HCOO]− | 872.5289 | PC(16:0/20:4 | x | x | x | x | PC(16:0/20:4[4 × DB, 1 × KETO, 1 × OOH]) |
| | | | | [4 × DB]) + 3O—2H | | | | | PC(16:0/20:4[4 × DB, 2 × OH, 1 × KETO]) |
| | | | | | | | | | PC(16:0/20:3[3 × DB, 1 × OH, 1 × KETO, 1 × |
| | | | | | | | | | EPOXY]) |
| | | | | | | | | | PC(16:0/20:2[2 × DB, 1 × KETO, 2 × EPOXY]) |
| | | | | | | | | | PC(16:0/[PGK2]) |
| | | | | | | | | | PC(16:0/[epoxyisoprostane D2]) |
| | | | | | | | | | PC(16:0/[epoxyisoprostane E2]) |
| | | | | | | | | | PC(16:0/[epoxyisoprostane H2]) |
| | | | | | | | | | PC(16:0/[epoxyisoprostane I2]) |
| | | | | | | | | | PC(16:0/[epoxyTXA]) |
| C44H80NO11P | 829.5469 | [M + HCOO]− | 874.5446 | PC(16:0/20:4 | x | x | x | x | PC(16:0/20:4[4 × DB, 1 × OH, 1 × OOH]) |
| | | | | [4 × DB]) + 3O | | | | | PC(16:0/20:4[4 × DB, 1 × OOH, 1 × EPOXY]) |
| | | | | | | | | | PC(16:0/20:4[4 × DB, 3 × OH]) |
| | | | | | | | | | PC(16:0/20:3[3 × DB, 2 × OH, 1 × EPOXY]) |
| | | | | | | | | | PC(16:0/20:2[2 × DB, 1 × OH, 2 × EPOXY]) |
| | | | | | | | | | PC(16:0/20:1[1 x DB, 3 × EPOXY]) |
| | | | | | | | | | PC(16:0/[PGD2]) |
| | | | | | | | | | PC(16:0/[PGE2]) |
| | | | | | | | | | PC(16:0/[PGH2]) |
| | | | | | | | | | PC(16:0/[PGI2]) |
| | | | | | | | | | PC(16:0/[TXA]) |
| C44H76NO12P | 841.5105 | [M + HCOO]− | 886.5082 | PC(16:0/20:4 | | | | x | PC(16:0/20:4[4 × DB, 2 × KETO, 1 × OOH]) |
| | | | | [4 × DB]) + 4O—4H | | | | | PC(16:0/20:4[4 × DB, 2 × OH, 2 × KETO]) |
| | | | | | | | | | PC(16:0/20:3[3 × DB, 1 × OH, 2 × KETO, 1 × |
| | | | | | | | | | EPOXY]) |
| | | | | | | | | | PC(16:0/20:2[2 × DB, 2 × KETO, 2 × EPOXY]) |
| C44H78NO12P | 843.5262 | [M + HCOO]− | 888.5238 | PC(16:0/20:4 | | x | | x | PC(16:0/20:4[4 × DB, 1 × OH, 1 × KETO,1 × |
| | | | | [4 × DB]) + 4O—2H | | | | | OOH]) |
| | | | | | | | | | PC(16:0/20:4[4 × DB, 3 × OH, 1 × KETO]) |
| | | | | | | | | | PC(16:0/20:3[4 × DB, 1 × KETO, 1 × OOH, |
| | | | | | | | | | 1 × EPOXY]) |
| | | | | | | | | | PC(16:0/20:3[3 × DB, 2 × OH, 1 × KETO, 1 × |
| | | | | | | | | | EPOXY]) |
| | | | | | | | | | PC(16:0/20:2[2 × DB, 1 × OH, 1 × KETO, 2 × |
| | | | | | | | | | EPOXY]) |
| | | | | | | | | | PC(16:0/20:1[1 × DB, 1 × KETO, 3 × EPOXY]) |
| C44H80NO12P | 845.5418 | [M + HCOO]− | 890.5395 | PC(16:0/20:4 | x | x | x | x | PC(16:0/20:4[4 × DB, 2 × OOH]) |
| | | | | [4 × DB]) + 4O | | | | | PC(16:0/20:4[4 × DB, 2 × OH, 1 × OOH]) |
| | | | | | | | | | PC(16:0/20:4[4 × DB, 4 × OH]) |
| | | | | | | | | | PC(16:0/20:3[3 × DB, 1 × OH, 1 × OOH, 1 × |
| | | | | | | | | | EPOXY]) |
| | | | | | | | | | PC(16:0/20:2[2 × DB, 1 × OOH, 2 × EPOXY]) |
| | | | | | | | | | PC(16:0/[PGG2]) |
| C44H78NO13P | 859.5211 | [M + HCOO]− | 904.5187 | PC(16:0/20:4 | x | x | x | x | PC(16:0/20:4[4 × DB, 1 × KETO, 2 × OOH]) |
| | | | | [4 × DB]) + 5O—2H | | | | | PC(16:0/20:4[4 × DB, 2 × OH, 1 × KETO, 1 × |
| | | | | | | | | | OOH]) |
| | | | | | | | | | PC(16:0/20:4[4 × DB, 1 × OH, 1 × KETO, 1 × |
| | | | | | | | | | OOH, 1 × EPOXY]) |
| | | | | | | | | | PC(16:0/20:4[4 × DB, 1 × KETO, 1 × OOH, 2 × |
| | | | | | | | | | EPOXY]) |
| | | | | | | | | | PC(16:0/20:4[4 × DB, 4 × OH, 1 × KETO]) |
| | | | | | | | | | PC(16:0/20:3[3 × DB,3 × OH, 1 × KETO, 1 × |
| | | | | | | | | | EPOXY]) |
| | | | | | | | | | PC(16:0/20:2[2 × DB, 2 × OH, 1 × KETO, 2 × |
| | | | | | | | | | EPOXY]) |
| | | | | | | | | | PC(16:0/20:l[1 × DB, 1 × OH, 1 × KETO, 3 × |
| | | | | | | | | | EPOXY]) |
| | | | | | | | | | PC(16:0/20:0[1 × OH, 1 × KETO, 4 × EPOXY]) |
| C44H80NO13P | 861.5367 | [M + HCOO]− | 906.5344 | PC(16:0/20:4 | x | | x | x | PC(16:0/20:4[4 × DB, 1 × OH, 2 × OOH]) |
| | | | | [4 × DB]) + 5O | | | | | PC(16:0/20:3[4 × DB, 2 × OOH, 1 × EPOXY]) |
| | | | | | | | | | PC(16:0/20:4[4 × DB, 3 × OH, 1 × OOH]) |
| | | | | | | | | | PC(16:0/20:3[3 × DB, 2 × OH, 1 × OOH, 1 × |
| | | | | | | | | | EPOXY]) |
| | | | | | | | | | PC(16:0/20:2[2 × DB, 1 × OH, 1 × OOH, 2 × |
| | | | | | | | | | EPOXY]) |
| | | | | | | | | | PC(16:0/20:1[1 × DB, 1 × OOH, 3 × EPOXY]) |
| C44H82NO13P | 863.5524 | [M + HCOO]− | 908.5500 | PC(16:0/20:3 | | | x | x | PC(16:0/20:3[3 × DB, 1 × OH, 2 × OOH]) |
| | | | | [3 × DB]) + 5O | | | | | PC(16:0/20:2[2 × DB, 2 × OOH, 1 × EPOXY]) |
| | | | | | | | | | PC(16:0/20:3[3 × DB, 3 × OH, 1 × OOH]) |
| | | | | | | | | | PC(16:0/20:2[2 × DB, 2 × OH, 1 × OOH, 1 × |
| | | | | | | | | | EPOXY]) |
| | | | | | | | | | PC(16:0/20:1[1 × DB, 1 × OH, 1 × OOH, 2 × |
| | | | | | | | | | EPOXY]) |
| | | | | | | | | | PC(16:0/20:0[1 × OOH, 3 × EPOXY]) |
| C44H80NO14P | 877.5316 | [M + HCOO]− | 922.5293 | PC(16:0/20:4 | x | x | x | x | PC(16:0/20:4[4 × DB, 3 × OOH]) |
| | | | | [4 × DB]) + 6O | | | | | PC(16:0/20:4[4 × DB, 2 × OH, 2 × OOH]) |
| | | | | | | | | | PC(16:0/20:3[3 × DB, 1 × OH, 2 × OOH, 1 × |
| | | | | | | | | | EPOXY]) |
| | | | | | | | | | PC(16:0/20:2[2 × DB, 2 × OOH, 2 × EPOXY]) |
| C44H80NO15P | 893.5266 | [M + HCOO]− | 938.5242 | PC(16:0/20:4 | | x | x | x | PC(16:0/20:4[4 × DB, 1 × OH, 3 × OOH]) |
| | | | | [4 × DB]) + 7O | | | | | PC(16:0/20:3[3 × DB, 3 × OOH, 1 × EPOXY]) |
| C44H80NO16P | 909.5215 | [M + HCOO]− | 954.5191 | PC(16:0/20:4 | | | x | x | PC(16:0/20:4[4 × DB, 4 × OOH]) |
| | | | | [4 × DB]) + 8O | | | | | PC(16:0/20:4[4 × DB, 2 × OH, 3 × OOH]) |
| | | | | | | | | | PC(16:0/20:3[3 × DB, 1 × OH, 3 × OOH, 1 × |
| | | | | | | | | | EPOXY]) |
| | | | | | | | | | PC(16:0/20:2[2 × DB, 3× OOH, 2 × EPOXY]) |

Nomenclature: “Lipid nomenclature is based on the LIPID MAPS consortium recommendations [31]. For instance, the shorthand notation PC 36:4 represents a phosphatidylcholine lipid containing 36 carbons and four double bonds. When the fatty acid identities and sn-position are known, as in our case, the slash separator is used (e.g., PC 16:0/20:4). Since no unified nomenclature is available for oxidized lipids, the short hand notations provided by LPPtiger tool were used [28]. Short chain oxidized lipids were indicated by the corresponding terminal enclosed in angular brackets (e.g. “<” and “>”), with the truncation site indicated by the carbon atom number (e.g., <COOH@C9> and <CHO@C12). For long chain products our recommendation is to indicate the number of oxygen addition after the fully identified parent lipid (e.g. PC 16:0/20:4 + 1O) when the type of addition is not known, or in parenthesis for known functional groups (e.g. PC 16:0/20:4[1 × OH@C11]).” Ni et al., “Evaluation of Air Oxidized PAPC: A Multi Laboratory Study by LC-MS/MS,” Free Radical Biology and Medicine 144:156-66 (2019) at 2.7.

The term “hyperactive dendritic cell” or “hyperactive macrophage” as used herein, refers to cells that have the ability to secrete interleukin-1 while maintaining viability. This process is typically associated with the assembly of inflammnasomes within the hyperactive cell.

“Optional” or “optionally” means that the subsequently described event or circumstance can or cannot occur, and that the description includes instances where the event or circumstance occurs and instances where it does not.

As used in this specification and the appended claims, the term “or” is generally employed in its sense including “and/or” unless the content clearly dictates otherwise.

The term “oxPAPC” or “oxidized PAPC”, as used herein, refers to lipids generated by the oxidation of 1-palmitoyl-2-arachidonyl-sn-glycero-3-phosphorylcholine (PAPC), which results in a mixture of oxidized phospholipids containing either fragmented or full length oxygenated sn-2 residues. Well-characterized oxidatively fragmented species contain a five-carbon sn-2 residue bearing omega-aldehyde or omega-carboxyl groups. Oxidation of arachidonic acid residue also produces phospholipids containing esterified isoprostanes. oxPAPC includes HOdiA-PC, KOdiA-PC, HOOA-PC, PGPC, POVPC and KOOA-PC species, among other oxidized products present in oxPAPC.

“Parenteral” administration of an immunogenic composition includes, e.g., subcutaneous (s.c.), intravenous (i.v.), intramuscular (i.m.), or intrasternal injection, or infusion techniques.

The terms “patient” or “individual” or “subject” are used interchangeably herein, and refers to a mammalian subject to be treated, with human patients being preferred. In some cases, the methods as described herein find use in experimental animals, in veterinary application, and in the development of animal models for disease, including, but not limited to, rodents including mice, rats, and hamsters, and primates.

As used herein, a “pharmaceutically acceptable” component/carrier etc. is one that is suitable for use with humans and/or animals without undue adverse side effects (such as toxicity, irritation, and allergic response) commensurate with a reasonable benefit/risk ratio.

A “suitable dosage level” refers to a dosage level that provides a therapeutically reasonable balance between pharmacological effectiveness and deleterious effects (e.g., sufficiently immunostimulatory activity imparted by an administered immunogen in the presence of an adjuvant lipid as described herein, with sufficiently low macrophage stimulation levels). For example, this dosage level can be related to the peak or average serum levels in a subject of, e.g., an anti-immunogen antibody produced following administration of an immunogenic composition (comprising an adjuvant lipid as described herein) at the particular dosage level.

To “treat” a disease as the term is used herein, means to reduce the frequency or severity of at least one sign or symptom of a disease or disorder, e.g. cancer, experienced by a subject.

“Treatment” is an intervention performed with the intention of preventing the development or altering the pathology or symptoms of a disorder. Accordingly, “treatment” refers to both therapeutic treatment and prophylactic or preventative measures. “Treatment” can also be specified as palliative care. Those in need of treatment include those already with the disorder as well as those in which the disorder is to be prevented. Accordingly, “treating” or “treatment” of a state, disorder or condition includes: (1) preventing or delaying the appearance of clinical symptoms of the state, disorder or condition developing in a human or other mammal that can be afflicted with or predisposed to the state, disorder or condition but does not yet experience or display clinical or subclinical symptoms of the state, disorder or condition; (2) inhibiting the state, disorder or condition, i.e., arresting, reducing or delaying the development of the disease or a relapse thereof (in case of maintenance treatment) or at least one clinical or subclinical symptom thereof; or (3) relieving the disease, i.e., causing regression of the state, disorder or condition or at least one of its clinical or subclinical symptoms. The benefit to an individual to be treated is either statistically significant or at least perceptible to the patient or to the physician.

As defined herein, a “therapeutically effective” amount of a compound or agent (i.e., an effective dosage) means an amount sufficient to produce a therapeutically (e.g., clinically) desirable result. The compositions can be administered from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds as described herein can include a single treatment or a series of treatments.

Genes: All genes, gene names, and gene products disclosed herein are intended to correspond to homologs from any species for which the compositions and methods disclosed herein are applicable. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, are intended to encompass homologous and/or orthologous genes and gene products from other species.

Ranges: throughout this disclosure, various aspects can be presented in a range format. It should be understood that the description in range format is merely for convenience and brevity and should not be construed as an inflexible limitation on the scope of the present claims. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Any compositions or methods provided herein can be combined with one or more of any of the other compositions and methods provided herein.

### Regulation of Dendritic Cells (DCs) and Pattern Recognition Receptors (PRRs)

The innate immune system has classically been viewed to operate in an all-or-none fashion, with DCs operating either to mount inflammatory responses that promote adaptive immunity, or not. Toll-like receptors (TLRs) expressed by DCs are therefore believed to be of central importance in determining immunogenic potential of these cells. The mammalian immune system is responsible for detecting microorganisms and activating protective responses that restrict infection. Central to this task are the dendritic cells, which sense microbes and subsequently promote T-cell activation. It has been suggested that dendritic cells can gauge the threat of any infection and instruct a proportional response (Blander, J. M. (2014). Nat Rev Immunol 14, 601-618; Vance, R. E. et al., (2009) Cell host & microbe 6, 10-21), but the mechanisms by which these immuno-regulatory activities could occur are unclear.

PRRs act to either directly or indirectly detect molecules that are common to broad classes of microbes. These molecules are classically referred to as pathogen associated molecular patterns (PAMPs), and include factors such as bacterial lipopolysaccharides (LPS), bacterial flagellin or viral double stranded RNA, among others.

An important attribute of PRRs as regulators of immunity is their ability to recognize specific microbial products. As such, PRR-mediated signaling events should provide a definitive indication of infection. It was postulated that a “GO” signal is activated by PRRs expressed on DCs that promote inflammation and T-cell mediated immunity. Interestingly, several groups have recently proposed that DCs can not simply operate in this all-or-none fashion (Blander, J. M., and Sander, L. E. (2012). Nat Rev Immunol 12, 215-225; Vance, R. E. et al., (2009) Cell host & microbe 6, 10-21). Rather, DCs can have the ability to gauge the threat (or virulence) that any possible infection poses and mount a proportional response. The most commonly discussed means by which virulence can be gauged is based on the ability of virulent pathogens to activate a greater diversity of PRRs than non-pathogens. However, not all microbes have a common set of PRR activators, and not all PRR activators are of comparable potency. The number of PRRs activated during an infection can therefore not be an ideal gauge of virulence. Moreover, increasing the number of PRRs activated during an infection will lead to a greater inflammatory response in general, which can indirectly promote greater T-cell responses. Conditions previously suggested to heighten the state of DC activation (e.g. through the use of virulent pathogens as stimuli) are also expected to heighten the state of MΦ activation (Vance, R. E. et al., (2009) Cell host & microbe 6, 10-21). Thus, it remains unclear if mechanisms are truly in place for the immune system (i.e. DCs) to gauge the threat of an infection specifically.

One possible means by which the threat of infection could be assessed would be through the well-recognized process of coincidence detection, where independent inputs result in a response that differs from the one elicited by any single input. In the context of PRRs, one such input must be a microbial product as an indicator of infection, regardless of the threat of virulence. In order to gauge the virulence threat, a second input must exist. Without wishing to be bound by theory, it is now thought that this putative second input is a molecule produced at the site of tissue injury, as cellular damage is often a feature associated with highly pathogenic microbes. Candidate molecules that can provide a second stimulus to DCs are the diverse family of molecules called danger associated molecules patterns (DAMPs), which are also known as alarmins (Kono, H., and Rock, K. L. (2008) Nat Rev Immunol 8, 279-289; Pradeu, T., and Cooper, E. L. (2012) Front Immunol 3, 287). DAMPs have been found at sites of infectious and non-infectious tissue injury, and have been proposed to modulate inflammatory responses, although their mechanisms of action remain unclear. One such class of DAMPs is represented by oxidized phospholipids derived from 1-palmitoyl-2-arachidonyl-sn-glycero-3-phosphorylcholine (PAPC), which are collectively known as oxPAPC. These lipids are produced at the sites of both infectious and non-infectious tissue injury (Berliner, J. A., and Watson, A. D. (2005). N Engl J Med 353, 9-11; Imai, Y. et al. (2008) Cell 133, 235-249; Shirey, K. A. et al. (2013) Nature 497, 498-502) and are found at very high levels in the membranes of dying cells (Chang, M. K. et al., (2004) J Exp Med 200, 1359-1370). oxPAPC is also an active component of oxidized low density lipoprotein (oxLDL) aggregates that promote inflammation in atherosclerotic tissues (Leitinger, N. (2003) Curr Opin Lipidol 14, 421-430), where local concentrations can be as high as 10-100 μM (Oskolkova, O. V. et al. (2010) J Immunol 185, 7706-7712). The association between oxPAPC and dying cells raised the possibility that these lipids could serve as a generic indicator of tissue health. In the presence of microbial product(s), oxPAPC can therefore indicate an increased infectious threat.

Due to the aforementioned features of activated DCs, these cells are well-equipped to stimulate antigen-specific T cell responses and numerous strategies have been undertaken to promote DC activation to drive protective immunity. These strategies commonly involve the use of synthetic or natural microbial products that stimulate PRRs of the Toll-like Receptor (TLR) family, with a notable example being the molecule monophosphoryl lipid A (MPLA), an FDA-approved TLR4 ligand that is used to adjuvant an increasing number of vaccines (J. Paavonen, Lancet, vol. 374, no. 9686, pp. 301-314, July 2009; M. Kundi, Expert Rev. Vaccines, vol. 6, no. 2, pp. 133-140, April 2007; A. M. Didierlaurent, et al. J. Immunol., vol. 183, no. 10, pp. 6186-6197, November 2009). Notably, TLRs alone do not upregulate all the molecular signals needed to promote T cell mediated immunity. Members of the interleukin-1 (IL-1) family of cytokines are critical regulators of many aspects of T cell differentiation, long-lived memory T cell generation and effector function (S. Z. Ben-Sasson, et al. Proc. Natl. Acad. Sci. U.S.A, vol. 106, no. 17, pp. 7119-24, April 2009; S. Z. Ben-Sasson, et al. J. Exp. Med., vol. 210, no. 3, pp. 491-502, March 2013; A. Jain, et al. Nat. Commun., vol. 9, no. 1, pp. 1-13, 2018). The expression of IL-1β, a well-characterized family member, is highly induced by TLR signals, but this cytokine lacks an N-terminal secretion signal and is therefore not released from cells via the conventional biosynthetic pathway. Rather, IL-1β accumulates in an inactive state in the cytosol of DCs that have been activated by TLR ligands (C. Garlanda, et al. Immunity, vol. 39, no. 6, pp. 1003-1018, December 2013). The lack of IL-1β release from activated DCs raises the possibility that TLR signals alone are not sufficient to maximally stimulate T cell responses and protective immunity.

The DC activation state is not the only cell fate DCs can achieve upon PRR signaling. Indeed, different PRRs stimulate distinct fates of these cells. One such fate is a commitment to an inflammatory form of cell death known as pyroptosis. Pyroptosis is a regulated process that results from the actions of inflammasomes, which are supramolecular organizing centers (SMOCs) that assemble in the cytosol of DCs and other cells (A. Lu, et al. Cell, vol. 156, no. 6, pp. 1193-1206, March 2014; J. C. Kagan, et al. Nat. Rev. Immunol., vol. 14, no. 12, pp. 821-826, December 2014). Inflammasome assembly is commonly stimulated upon detection of PAMPs or DAMPs in the cytosol of the host cell and as such, cytosolic PRRs are responsible for linking threat assessment in the cytosol to inflammasome-dependent pyroptosis (K. J. Kieser and J. C. Kagan, Nat. Rev. Immunol., vol. 17, no. 6, pp. 376-390, May 2017; M. Lamkanfi and V. M. Dixit, Cell, vol. 157, no. 5, pp. 1013-22, May 2014). The process of pyroptosis leads to the release of IL-1β and other IL-1 family members from the cell, therefore providing the signal to T cells that TLRs cannot offer. Despite this gain in activity, in terms of promoting IL-1β release, pyroptotic cells are dead and have therefore lost the ability to participate in the days-long process needed to stimulate and differentiate naïve T cells in dLN (T. R. Mempel, et al. Nature, vol. 427, no. 6970, pp. 154-159, January 2004). Indeed, stimuli that promote pyroptosis, such as the commonly used vaccine adjuvant alum (S. C. Eisenbarth, et al. Nature, vol. 453, no. 7198, pp. 1122-1126, June 2008; M. Kool, et al. J. Immunol., vol. 181, no. 6, pp. 3755-3759, September 2008), are best-appreciated for their ability to stimulate type 2 immune responses (P. Marrack, et al. Nat. Rev. Immunol., vol. 9, no. 4, pp. 287-293, April 2009), which are not suited for the elimination of many microbial infections or cancers.

In certain embodiments, a method of treating cancer, comprises administering to a subject in need thereof, a therapeutically effective amount of a composition comprising dendritic cell hyperactivating stimuli along with tumor cell lysates that serve as immunogens, thereby treating cancer. In certain embodiments, the method further comprises administering a chemotherapeutic agent, an immunogen or a combination thereof.

In certain embodiments, the hyperactivating stimuli, the chemotherapeutic agent, the immunogen or combinations thereof, are co-administered or sequentially administered.

In certain embodiments, the hyperactivating stimulus includes a combination of a pattern recognition receptor ligand and 1-palmityl-2-(5-glutaryl)-sn-glycero-3-phosphocholine (PGPC).

In certain embodiments, dendritic cell hyperactivating stimulus comprises a pattern recognition receptor ligand and 1-palmitoyl-2-arachidonyl-sn-glycero-3-phosphorylcholine (PAPC), oxidized 1-palmitoyl-2-arachidonoyl-sn-glycero-3-phosphorylcholine (oxPAPC), species of oxPAPC, components thereof or combinations thereof.

TLR4 Ligands

In some embodiments of the methods described herein, the TLR ligand is selected from a TLR1 ligand, a TLR2 ligand, a TLR3 ligand, a TLR4 ligand, a TLR5 ligand, a TLR6 ligand, a TLR7 ligand, a TLR8 ligand, a TLR9 ligand, a TLR10 ligand, a TLR11 ligand, a TLR12 ligand, a TLR13 ligand, and combinations thereof.

In some embodiments of the methods described herein, the TLR ligand is a TLR4 ligand.

In some embodiments of the methods described herein, the TLR4 ligand is selected from monophosphoryl lipid A (MPLA), lipopolysaccharide (LPS), or combinations thereof.

### Immunogens

Immunogens, e.g., cancer immunogens, and their use, e.g., in cancer vaccines, described in the art. See. e.g., Michael J. P. Lawman and Patricia D. Lawman (eds.) “Cancer Vaccines, Methods and Protocols” Methods in Molecular Biol. 1136 (2014); Chiang et al., “Whole Tumor Antigen Vaccines: Where Are We?” Vaccines (Basel) 3(2):344-72 (2015); Thumann et al., “Antigen Loading of Dendritic Cells with Whole Tumor Cell Preparations,” J. Immunol. Methods 277:1-16 (2003); Kamigaki et al., “Immunotherapy of Autologous Tumor Lysate-Loaded Dendritic Cell Vaccines by a Closed-Flow Electroporation System for Solid Tumors,” Anticancer Res. 33:2971-6 (2013); U.S. Pat. Nos. 3,823,126A; 3,960,827A; and 4,160,018A.

In some embodiments, the immunogen is a cancer antigen. In some embodiments, the cancer antigen is selected from a tumor lysate, an apoptotic body, a peptide, a tumor RNA, a tumor derived exosome, a tumor-DC fusion, or combinations thereof.

In some embodiments, the immunogen is a whole tumor lysate.

In some embodiments, the whole tumor lysate is prepared by irradiating, boiling, and or freeze-thaw lysis.

In some embodiments, the immunogen is autologous. In some embodiments, the immunogen is allogenic.

In some embodiments of methods of inducing an immune response in a subject, the immunogen is a tumor lysate derived from the cell donor.

In some embodiments of the methods described herein, the cancer immunogen is an infectious agent immunogen, wherein an infection with the infectious agent is associated with development of cancer.

In some embodiments of the methods described herein, the cancer immunogen is from a cancer immunogen cell.

In some embodiments of the methods described herein, the cancer immunogen is or comprises whole tumor cell lysate.

Immunogenic compositions comprising adjuvants as described herein can be administered to a subject using any known form of vaccine, e.g., tumor antigens, tumor cell lysates, attenuated virus, protein, nucleic acid, etc. vaccine, so as to produce in the subject, an amount of the selected immunogen which is effective in inducing a therapeutic or prophylactic immune response against the target antigen in the subject. The subject can be a human or nonhuman subject. Animal subjects include, without limitation, non-human primates, dogs, cats, equines (horses), ruminants (e.g., sheep, goats, cattle, camels, alpacas, llamas, deer), pigs, birds (e.g., chicken, turkey quail), rodents, and chirodoptera. Subjects can be treated for any purpose, including without limitation, eliciting a protective immune response, or producing antibodies (or B cells) for collection and use for other purposes.

An immunogen of interest is expressed by diseased target cells (e.g., neoplastic cell, infected cells), and expressed in lower amounts or not at all in other tissue. Examples of target cells include cells from a neoplastic disease, including but not limited to sarcoma, lymphoma, leukemia, a carcinoma, melanoma, carcinoma of the breast, carcinoma of the prostate, ovarian carcinoma, carcinoma of the cervix, colon carcinoma, carcinoma of the lung, glioblastoma, and astrocytoma. Alternatively, the target cell can be infected by, for example, a virus, a mycoplasma, a parasite, a protozoan, a prion and the like. Accordingly, an immunogen of interest can be from, without limitation, a human papilloma virus (see below), a herpes virus such as herpes simplex or herpes zoster, a retrovirus such as human immunodeficiency virus 1 or 2, a hepatitis virus, an influenza virus, a rhinovirus, respiratory syncytial virus, cytomegalovirus, adenovirus, Mycoplasma pneumoniae, a bacterium of the genus Salmonella, Staphylococcus, Streptococcus, Enterococcus, Clostridium, Escherichia, Klebsiella, Vibrio, Mycobacterium, amoeba, a malarial parasite, and Trypanosoma cruzi.

In addition to tumor antigens, tumor cell lysates and antigens of infectious agents, mutants of tumor suppressor gene products including, but not limited to, p53, BRCA1, BRCA2, retinoblastoma, and TSG101, or oncogene products such as, without limitation, RAS, W T, MYC, ERK, and TRK, can also provide target antigens to be used according to the present disclosure. The target antigen can be a self-antigen, for example one associated with a cancer or neoplastic disease. In some embodiments, the immunogen is a peptide from a heat shock protein (hsp)-peptide complex of a diseased cell, or the hsp-peptide complex itself.

Immunogenic compositions as described herein can comprise an immunogen and an adjuvant lipid, and can be administered for therapeutic and/or prophylactic purposes. In therapeutic applications, an immunogenic composition as described herein is administered in an amount sufficient to elicit an effective immune response and/or hyperactivate dendritic cells to treat a disease or arrest progression and/or symptoms. The dosage of the adjuvants as described herein can vary depending on the nature of the immunogen and the condition of the subject, but should be sufficient to enhance the efficacy of the immunogen in evoking an immunogenic response. For therapeutic or prophylactic treatment, the amount of adjuvant administered can range from 0.05, 0.1, 0.5, or 1 mg per kg body weight, up to about 10, 50, or 100 mg per kg body weight or more. The adjuvants as described herein are generally non-toxic, and generally can be administered in relatively large amount without causing life-threatening side effects.

The term “therapeutic immune response”, as used herein, refers to an increase in humoral and/or cellular immunity, as measured by standard techniques, which is directed toward the target antigen. Preferably, the induced level of immunity directed toward the target antigen is at least four times, and preferably at least 5 times the level prior to the administration of the immunogen. The immune response can also be measured qualitatively, wherein by means of a suitable in vitro or in vivo assay, an arrest in progression or a remission of a neoplastic or infectious disease in the subject is considered to indicate the induction of a therapeutic immune response.

In some embodiments of the methods as described herein, a composition comprising an immunogen and an adjuvant as described herein, combined in therapeutically effective amounts, is administered to a mammal in need thereof. The term “administering” as used herein means delivering the immunogen and adjuvant as described herein to a mammal by any method that can achieve the result sought. They can be administered, for example, intravenously or intramuscularly. The term “mammal” as used herein is intended to include, but is not limited to, humans, laboratory animals, domestic pets and farm animals. “Therapeutically effective amount” means an amount of the immunogen and adjuvant that, when administered to a mammal, is effective in producing the desired therapeutic effect.

Compositions comprising immunogens and adjuvants as described herein can be administered cutaneously, subcutaneously, intravenously, intramuscularly, parenterally, intrapulmonarily, intravaginally, intrarectally, nasally or topically. The composition can be delivered by injection, orally, by aerosol, or particle bombardment.

Compositions for administration can further include various additional materials, such as a pharmaceutically acceptable carrier. Suitable carriers include any of the standard pharmaceutically accepted carriers, such as phosphate buffered saline solution, water, emulsions such as an oil/water emulsion or a triglyceride emulsion, various types of wetting agents, tablets, coated tablets and capsules. Typically such carriers contain excipients such as starch, milk, sugar, certain types of clay, gelatin, stearic acid, talc, vegetable fats or oils, gums, glycols, or other known excipients. Such carriers can also include flavor and color additives or other ingredients. The compositions described herein can also include suitable diluents, preservatives, solubilizers, emulsifiers, adjuvants and/or carriers. Such compositions can be in the form of liquid or lyophilized or otherwise dried formulations and can include diluents of various buffer content (e.g., Tris-HCl, acetate, phosphate), pH and ionic strength, additives such as albumin or gelatin to prevent absorption to surfaces, detergents (e.g., Tween 20, Tween 80, Pluronic F68, bile acid salts), solubilizing agents (e.g. glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexing with metal ions, or incorporation of the material into or onto particulate preparations of polymeric compounds such as polylactic acid, polyglycolic acid, hydrogels, etc. or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance.

Combination Therapies

In certain embodiments it may be preferable to administer to a subject, one or more other agents which are of therapeutic benefit. These include without limitation, chemotherapeutic agents, compounds, cytokine antagonist, cytokine receptor antagonist, cytokines, adoptive cell therapy, anti-viral agents, checkpoint inhibitors, adjuvants or combinations thereof. In certain embodiments, compounds comprise at least one amidoamine compound. Amidoamines are a class of chemical compounds that are formed from fatty acids and diamines. An example of an amidoamine compound is myristamidopropyl dimethylamine (Aldox).

Immune Checkpoint Modulation: In certain embodiments, immune checkpoint modulators are co-administered with the hyperactivated dendritic cells. Immune checkpoints refer to inhibitory pathways of the immune system that are responsible for maintaining self-tolerance and modulating the duration and amplitude of physiological immune responses.

Certain cancer cells thrive by taking advantage of immune checkpoint pathways as a major mechanism of immune resistance, particularly with respect to T cells that are specific for tumor antigens. For example, certain cancer cells overexpress one or more immune checkpoint proteins responsible for inhibiting a cytotoxic T cell response. Thus, immune checkpoint modulators can be administered to overcome the inhibitory signals and permit and/or augment an immune attack against cancer cells. Immune checkpoint modulators can facilitate immune cell responses against cancer cells by decreasing, inhibiting, or abrogating signaling by negative immune response regulators (e.g. CTLA4), or can stimulate or enhance signaling of positive regulators of immune response (e.g. CD28).

Immunotherapy agents targeted to immune checkpoint modulators can be administered to encourage immune attack targeting cancer cells. Immunotherapy agents can be or include antibody agents that target (e.g., are specific for) immune checkpoint modulators. Examples of immunotherapy agents include antibody agents targeting one or more of CTLA-4, PD-1, PD-L1, GITR, OX40, LAG-3, KIR, TIM-3, CD28, CD40; and CD137. Specific examples of antibody agents can include monoclonal antibodies. Certain monoclonal antibodies targeting immune checkpoint modulators are available. For instance, ipilumimab targets CTLA-4; tremelimumab targets CTLA-4; pembrolizumab targets PD-1, etc.

The Programmed Death 1 (PD-1) protein is an inhibitory member of the extended CD28/CTLA-4 family of T cell regulators (Okazaki et al. (2002) Curr Opin Immunol 14: 391779-82; Bennett et al. (2003) J. Immunol. 170:711-8). Other members of the CD28 family include CD28, CTLA-4, ICOS and BTLA. Two cell surface glycoprotein ligands for PD-1 have been identified, Program Death Ligand 1 (PD-L1) and Program Death Ligand 2 (PD-L2). PD-L1 and PD-L2 have been shown to downregulate T cell activation and cytokine secretion upon binding to PD-1 (Freeman et al. (2000) J Exp Med 192:1027-34; Latchman et al. (2001) Nat Immunol 2:261-8; Carter et al. (2002) Eur J Immunol 32:634-43; Ohigashi et al. (2005) Clin Cancer Res 11:2947-53).

PD-L1 (also known as cluster of differentiation 274 (CD274) or B7 homolog 1 (B7-H1)) is a 40 kDa type 1 transmembrane protein. PD-L1 binds to its receptor, PD-1, found on activated T cells, B cells, and myeloid cells, to modulate activation or inhibition. Both PD-L1 and PD-L2 are B7 homologs that bind to PD-1, but do not bind to CD28 or CTLA-4 (Blank et al. (2005) Cancer Immunol Immunother. 54:307-14). Binding of PD-L1 with its receptor PD-1 on T cells delivers a signal that inhibits TCR-mediated activation of IL-2 production and T cell proliferation. The mechanism involves inhibition of ZAP70 phosphorylation and its association with CD3.zeta. (Sheppard et al. (2004) FEBS Lett. 574:37-41). PD-1 signaling attenuates PKC-θ activation loop phosphorylation resulting from TCR signaling, necessary for the activation of transcription factors NF-κB and AP-1, and for production of IL-2. PD-L1 also binds to the costimulatory molecule CD80 (B7-1), but not CD86 (B7-2) (Butte et al. (2008) Mol Immunol. 45:3567-72).

Expression of PD-L1 on the cell surface has been shown to be upregulated through IFN-γ stimulation. PD-L1 expression has been found in many cancers, including human lung, ovarian and colon carcinoma and various myelomas, and is often associated with poor prognosis (Iwai et al. (2002) PNAS 99:12293-7; Ohigashi et al. (2005) Clin Cancer Res 11:2947-53; Okazaki et al. (2007) Intern. Immun. 19:813-24; Thompson et al. (2006) Cancer Res. 66:3381-5). PD-L1 has been suggested to play a role in tumor immunity by increasing apoptosis of antigen-specific T-cell clones (Dong et al. (2002) Nat Med 8:793-800). It has also been suggested that PD-L1 might be involved in intestinal mucosal inflammation and inhibition of PD-L1 suppresses wasting disease associated with colitis (Kanai et al. (2003) J Immunol 171:4156-63).

Exemplary anti-PD1 antibodies include pembrolizumab (MK-3475, Merck), nivolumab (BMS-936558, Bristol-Myers Squibb), and pidilizumab (CT-011, Curetech LTD.). Anti-PD1 antibodies are commercially available, for example from ABCAM™ (AB137132), BIOLEGEND™ (EH12.2H7, RMP1-14) and Affymetrix Ebioscience (J105, J116, MIH4).

Anti-Cancer Agents: In certain embodiments, the method further comprises administering an anti-cancer agent. In some embodiments, the anti-cancer agent is a chemotherapeutic or growth inhibitory agent, a targeted therapeutic agent, a T cell expressing a chimeric antigen receptor, an antibody or antigen-binding fragment thereof, an antibody-drug conjugate, an angiogenesis inhibitor, an antineoplastic agent, a cancer vaccine, an adjuvant, and combinations thereof.

In some embodiments, the anti-cancer agent is a chemotherapeutic or growth inhibitory agent. For example, a chemotherapeutic or growth inhibitory agent can include an alkylating agent, an anthracycline, an anti-hormonal agent, an aromatase inhibitor, an anti-androgen, a protein kinase inhibitor, a lipid kinase inhibitor, an antisense oligonucleotide, a ribozyme, an antimetabolite, a topoisomerase inhibitor, a cytotoxic agent or antitumor antibiotic, a proteasome inhibitor, an anti-microtubule agent, an EGFR antagonist, a retinoid, a tyrosine kinase inhibitor, a histone deacetylase inhibitor, and combinations thereof.

Examples of chemotherapeutic agents can include erlotinib (TARCEVA™, Genentech/OSI Pharm.), bortezomib (VELCADE™, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX™, AstraZeneca), sunitib (SUTENT™, Pfizer/Sugen), letrozole (FEMARA™, Novartis), imatinib mesylate (GLEEVEC™, Novartis), finasunate (VATALANIB™, Novartis), oxaliplatin (ELOXATIN™, Sanofi), 5-FU (5-fluorouracil), leucovorin, Rapamycin (Sirolimus, RAPAMUNE™, Wyeth), Lapatinib (TYKERB™, GSK572016, Glaxo Smith Kline), Lonafamib (SCH 66336), sorafenib (NEXAVAR™, Bayer Labs), gefitinib (IRESSA™, AstraZeneca), AG1478, alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5a-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; antibiotics such as the enediyne antibiotics (e.g., calicheamicin, especially calicheamicin γ1I and calicheamicin .omega.II (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN™ (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSKT™ polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE™ (Cremophor-free), albumin-engineered nanoparticle formulations of paclitaxel (American Pharmaceutical Partners, Schaumberg, Ill.), and TAXOTERE™ (docetaxel, doxetaxel; Sanofi-Aventis); chloranmbucil; GEMZAR™ (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE™ (vinorelbine); novantrone; teniposide; edatrexate; daunomycin; aminopterin; capecitabine (XELODA™); ibandronate; CPT-11; topoisomerase inhibitor RFS 2000; difluoromethylomithine (DMFO); retinoids such as retinoic acid; and pharmaceutically acceptable salts, acids and derivatives of any of the above.

In some embodiments, a chemotherapeutic agent can include alkylating agents (including monofunctional and bifunctional alkylators) such as thiotepa, CYTOXAN™ cyclosphosphamide, nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; temozolomide; and pharmaceutically acceptable salts, acids and derivatives of any of the above.

In some embodiments, a chemotherapeutic agent can include anthracyclines such as daunorubicin, doxorubicin, epirubicin, idarubicin, mitoxantrone, valrubicin, and pharmaceutically acceptable salts, acids and derivatives of any of the above.

In some embodiments, a chemotherapeutic agent can include an anti-hormonal agent such as anti-estrogens and selective estrogen receptor modulators (SERMs), including, for example, tamoxifen (including NOLVADEX™; tamoxifen citrate), raloxifene, droloxifene, iodoxyfene, 4-hydroxytamoxifen, trioxifene, keoxifene, LY117018, onapristone, and FARESTON™ (toremifine citrate); and pharmaceutically acceptable salts, acids and derivatives of any of the above.

In some embodiments, a chemotherapeutic agent can include an aromatase inhibitor that inhibit the enzyme aromatase, which regulates estrogen production in the adrenal glands, such as, for example, 4(5)-imidazoles, aminoglutethimide, MEGASE™ (megestrol acetate), AROMASIN™ (exemestane; Pfizer), formestanie, fadrozole, RIVISOR™ (vorozole), FEMARA™ (letrozole; Novartis), and ARIMIDEX™ (anastrozole; AstraZeneca); and pharmaceutically acceptable salts, acids and derivatives of any of the above.

In some embodiments, a chemotherapeutic agent can include an anti-androgen such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); and pharmaceutically acceptable salts, acids and derivatives of any of the above.

In some embodiments, a chemotherapeutic agent can include a protein kinase inhibitors, lipid kinase inhibitor, or an antisense oligonucleotide, particularly those which inhibit expression of genes in signaling pathways implicated in aberrant cell proliferation, such as, for example, PKC-alpha, Ralf and H-Ras.

In some embodiments, a chemotherapeutic agent can include a ribozyme such as VEGF expression inhibitors (e.g., ANGIOZYME™) and HER2 expression inhibitors.

In some embodiments, a chemotherapeutic agent can include a cytotoxic agent or antitumor antibiotic, such as dactinomycin, actinomycin, bleomycins, plicamycin, mitomycins such as mitomycin C, and pharmaceutically acceptable salts, acids and derivatives of any of the above.

In some embodiments, a chemotherapeutic agent can include a proteasome inhibitor such as bortezomib (VELCADE™, Millennium Pharm.), epoxomicins such as carfilzomib (KYPROLIS™, Onyx Pharm.), marizomib (NPI-0052), MLN2238, CEP-18770, oprozomib, and pharmaceutically acceptable salts, acids and derivatives of any of the above.

In some embodiments, a chemotherapeutic agent can include an anti-microtubule agent such as Vinca alkaloids, including vincristine, vinblastine, vindesine, and vinorelbine; taxanes, including paclitaxel and docetaxel; podophyllotoxin; and pharmaceutically acceptable salts, acids and derivatives of any of the above.

In some embodiments, a chemotherapeutic agent can include an “EGFR antagonist,” which refers to a compound that binds to or otherwise interacts directly with EGFR and prevents or reduces its signaling activity, and is alternatively referred to as an “EGFR i.” Examples of such agents include antibodies and small molecules that bind to EGFR.

Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6. 3 and E7.6. 3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody can be conjugated with a cytotoxic agent, thus generating an immunoconjugate (see, e.g., EP659439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA™ Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quin-azolinyl]-, dihydrochloride, Pfizer Inc.); ZD1839, gefitinib (IRESSA™) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol)-; (R)-6-(4-hydroxyphenyl)-4-[(1-phenylethyl)amino]-7H-pyrrolo[2,3-d]pyrimi-dine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(-dimethylamino)-2-butenamide) (Wyeth); AG1478 (Pfizer); AG1571 (SU 5271; Pfizer); dual EGFR/HER2 tyrosine kinase inhibitors such as lapatinib (TYKERB™, GSK572016 or N-[3-chloro-4-[(3 fluorophenyl)methoxy]phenyl]-6[5[[[2methylsulfonyl)ethyl]amino]methyl]-2-furanyl]-4-quinazolinamine).

In some embodiments, a chemotherapeutic agent can include a tyrosine kinase inhibitor, including the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC™, available from Glaxo SmithKline); multi-targeted tyrosine kinase inhibitors such as sunitinib (SUTENT™, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035, 4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d] pyrimidines; curcumin (diferuloyl methane, 4,5-bis (4-fluoroanilino)phthalimide); tyrphostines containing nitrothiophene moieties; PD-0183805 (Wamer-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC™); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE™); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

In some embodiments, a chemotherapeutic agent can include a retinoid such as retinoic acid and pharmaceutically acceptable salts, acids and derivatives of any of the above.

In some embodiments, a chemotherapeutic agent can include an anti-metabolite. Examples of anti-metabolites can include folic acid analogs and antifolates such as denopterin, methotrexate, pteropterin, trimetrexate; purine analogs such as fludarabine, 6-mercaptopurine, thiamiprine, thioguanine; pyrimidine analogs such as 5-fluorouracil (5-FU), ancitabine, azacitidine, 6-azauridine, carmofur, cytarabine, dideoxyuridine, doxifluridine, enocitabine, floxuridine; nucleoside analogs; and nucleotide analogs.

In some embodiments, a chemotherapeutic agent can include a topoisomerase inhibitor. Examples of topoisomerase inhibitors can include a topoisomerase 1 inhibitor such as LURTOTECAN™ and ABARELIX™ rmRH; a topoisomerase II inhibitor such as doxorubicin, epirubicin, etoposide, and bleomycin; and topoisomerase inhibitor RFS 2000.

In some embodiments, a chemotherapeutic agent can include a histone deacetylase (HDAC) inhibitor such as vorinostat, romidepsin, belinostat, mocetinostat, valproic acid, panobinostate, and pharmaceutically acceptable salts, acids and derivatives of any of the above.

Chemotherapeutic agents can also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFα) blockers such as etanercept (Enbrel), infliximab (Remicade), adalimumab (Humira), certolizumab pegol (Cimzia), golimumab (Simponi), Interleukin 1 (IL-1) blockers such as anakinra (Kineret), T cell costimulation blockers such as abatacept (Orencia), Interleukin 6 (IL-6) blockers such as tocilizumab (ACTEMERA™); Interleukin 13 (IL-13) blockers such as lebrikizumab; Interferon alpha (IFN) blockers such as Rontalizumab; Beta 7 integrin blockers such as rhuMAb Beta7; IgE pathway blockers such as Anti-M1 prime; Secreted homotrimeric LTa3 and membrane bound heterotrimer LTa1/β2 blockers such as Anti-lymphotoxin alpha (LTa); radioactive isotopes (e.g., ²¹¹At, ¹³¹I, ¹²⁵I, ⁹⁰Y, ¹⁸⁶Re, ¹⁸⁸Re, ²¹²Bi, ³²P, ²¹²Pb and radioactive isotopes of Lu); miscellaneous investigational agents such as thioplatin, PS-341, phenylbutyrate, ET-18-OCH3, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL™); beta-lapachone; lapachol; colchicines; betulinic acid; acetylcamptothecin, scopolectin, and 9-aminocamptothecin); podophyllotoxin; tegafur (UFTORAL™); bexarotene (TARGRETIN™); bisphosphonates such as clodronate (for example, BONEFOS™ or OSTAC™), etidronate (DIDROCAL™), NE-58095, zoledronic acid/zoledronate (ZOMETA™), alendronate (FOSAMAX™), pamidronate (AREDIA™), tiludronate (SKELID™), or risedronate (ACTONEL™); and epidermal growth factor receptor (EGF-R); vaccines such as THERATOPE™ vaccine; perifosine, COX-2 inhibitor (e.g. celecoxib or etoricoxib), proteosome inhibitor (e.g. PS341); CCI-779; tipifamib (R11577); orafenib, ABT510; Bcl-2 inhibitor such as oblimersen sodium (GENASENSE™); pixantrone; farnesyltransferase inhibitors such as lonafamib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents can also include non-steroidal anti-inflammatory drugs with analgesic, antipyretic and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lomoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhoea, metastatic bone pain, headache and migraine, postoperative pain, mild-to-moderate pain due to inflammation and tissue injury, pyrexia, ileus, and renal colic.

Pharmaceutical Therapeutics

In one aspect, the pharmaceutical compositions are administered systemically, for example, formulated in a pharmaceutically-acceptable buffer such as physiological saline. Preferable routes of administration include, for example, instillation into the bladder, subcutaneous, intravenous, intraperitoneal, intramuscular, intratumoral or intradermal injections that provide continuous, sustained or effective levels of the composition in the patient. Treatment of human patients or other animals is carried out using a therapeutically effective amount of a therapeutic identified herein in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the cancer. Generally, amounts will be in the range of those used for other agents used in the treatment of other diseases associated with cancer, although in certain instances lower amounts will be needed because of the increased specificity of the compound. A compound is administered at a dosage that enhances an immune response of a subject, or that reduces the proliferation, survival, or invasiveness of a neoplastic or, infected cell as determined by a method known to one skilled in the art.

The administration of compositions embodied herein, is by any suitable means that results in a concentration of the therapeutic that, combined with other components, is effective in ameliorating, reducing, or stabilizing cancer. The composition can be provided in a dosage form that is suitable for parenteral (e.g., subcutaneous, intravenous, intramuscular, intravesicular, intratumoral or intraperitoneal) administration route. For example, the pharmaceutical compositions are formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Human dosage amounts are initially determined by extrapolating from the amount of compound used in mice or non-human primates, as a skilled artisan recognizes it is routine in the art to modify the dosage for humans compared to animal models. For example, the dosage can vary from between about 1 sg compound/kg body weight to about 5000 mg compound/kg body weight; or from about 5 mg/kg body weight to about 4,000 mg/kg body weight or from about 10 mg/kg body weight to about 3,000 mg/kg body weight; or from about 50 mg/kg body weight to about 2000 mg/kg body weight; or from about 100 mg/kg body weight to about 1000 mg/kg body weight; or from about 150 mg/kg body weight to about 500 mg/kg body weight. For example, the dose is about 1, 5, 10, 25, 50, 75, 100, 150, 200, 250, 300, 350, 400, 450, 500, 550, 600, 650, 700, 750, 800, 850, 900, 950, 1,000, 1,050, 1,100, 1,150, 1,200, 1,250, 1,300, 1,350, 1,400, 1,450, 1,500, 1,600, 1,700, 1,800, 1,900, 2,000, 2,500, 3,000, 3,500, 4,000, 4,500, or 5,000 mg/kg body weight. Alternatively, doses are in the range of about 5 mg compound/Kg body weight to about 20 mg compound/kg body weight. In another example, the doses are about 8, 10, 12, 14, 16 or 18 mg/kg body weight. Of course, this dosage amount can be adjusted upward or downward, as is routinely done in such treatment protocols, depending on the results of the initial clinical trials and the needs of a particular patient.

Pharmaceutical compositions are formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

The pharmaceutical compositions embodied herein are administered parenterally by injection, infusion or implantation (subcutaneous, intravenous, intramuscular, intratumoral, intravesicular, intraperitoneal) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

In certain embodiments, the composition is in the form of a solution, a suspension, an emulsion, an infusion device, or a delivery device for implantation, or is presented as a dry powder to be reconstituted with water or another suitable vehicle before use. Apart from the active agent that reduces or ameliorates a cancer, the composition includes suitable parenterally acceptable carriers and/or excipients. The active therapeutic agent(s) can be incorporated into microspheres, microcapsules, nanoparticles, liposomes for controlled release. Furthermore, the composition can include suspending, solubilizing, stabilizing, pH-adjusting agents, tonicity adjusting agents, and/or dispersing, agents.

As indicated above, the pharmaceutical compositions can be in a form suitable for sterile injection. To prepare such a composition, the suitable active therapeutic(s) are dissolved or suspended in a parenterally acceptable liquid vehicle. Among acceptable vehicles and solvents that can be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation can also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent can include 10-60% w/w of propylene glycol.

Provided herein are methods of treating cancer or symptoms thereof that comprise administering a therapeutically effective amount of a pharmaceutical composition. Thus, described herein are methods of treating a subject suffering from or susceptible to a cancer. The methods can include a step of administering to the mammal a therapeutic amount of the compositions described herein, in a dose sufficient to treat the disease or disorder or symptom thereof, under conditions such that the disease or disorder is treated.

The methods herein can include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method). The therapeutic methods as described herein (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for cancer or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The fusion protein complexes as described herein can be used in the treatment of any other disorders in which an increase in an immune response is desired.

Also provided herein are methods of monitoring treatment progress. The methods can include a step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with cancer in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In some cases, a second level of Marker in the subject is determined at a time point later than the determination of the first level, and the two levels are compared to monitor the course of disease or the efficacy of the therapy. In certain aspects, a pre-treatment level of Marker in the subject is determined prior to beginning treatment as described herein; this pre-treatment level of Marker can then be compared to the level of Marker in the subject after the treatment commences, to determine the efficacy of the treatment.

The pharmaceutical compositions can be included in a kit, container, pack, or dispenser together with instructions for administration.

The practice of the present methods employ, unless otherwise indicated, known techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. It is to be understood and expected that variations in the principles of invention herein disclosed can be made by one skilled in the art and it is intended that such modifications are to be included within the scope of the present invention.

### EXEMPLARY EMBODIMENTS

1. A method of preparing an immunogenic composition, the method comprising:

a) depleting leukocytes from a suspension of cells prepared from a tumor to obtain a tumor cell-enriched suspension;

b) lysing cells from the tumor cell-enriched suspension to obtain a tumor cell lysate;

c) contacting the tumor cell lysate with an oxidized lipid and a toll-like receptor 4 (TLR4) agonist to obtain the immunogenic composition.

2. The method of embodiment 1, wherein the leukocytes are depleted in step a) by negative selection using an anti-CD45 antibody.

3. The method of embodiment 1, wherein the cells are lysed in step b) by one or more freeze-thaw cycles.

4. The method of embodiment 1, wherein the oxidized lipid comprises at least one phospholipid of oxidized 1-palmitoyl-2-arachidonyl-sn-glycero-3-phosphorylcholine (oxPAPC).

5. The method of embodiment 4, wherein the at least one phospholipid comprises at least one of the group consisting of POVPC, PGPC, PECPC, and PEIPC, optionally wherein the at least one phospholipid comprises PGPC.

6. The method of embodiment 1, wherein the TLR4 agonist comprises monophosphoryl lipid A (MPLA).

7. The method of embodiment 6, wherein the TLR4 agonist is present within an adjuvant, and the adjuvant further comprises one or more of an aluminum salt, a saponin, and liposomes, optionally wherein the adjuvant is AS01B or AS04.

8. The method of embodiment 1, further comprises before step a) obtaining a sample from the tumor and preparing the suspension of cells.

9. An immunogenic composition prepared by the method of any one of embodiments 1-8.

10. A method of eliciting an anti-cancer immune response, the method comprising:

administering to a mammalian subject with cancer an effective amount of the immunogenic composition of embodiment 9.

11. The method of embodiment 10, wherein the anti-cancer immune response comprises cellular immune response.

12. The method of embodiment 10, wherein the anti-cancer immune response comprises cancer antigen-induced IL-1beta secretion and activation of CD8+ T lymphocytes.

13. The method of any one of embodiments 10-12, wherein the cancer is a non-hematologic cancer.

14. The method of embodiment 13, wherein the non-hematologic cancer is a carcinoma, a sarcoma, or a melanoma.

15. The method of any one of embodiments 10-12, wherein the cancer is a lymphoma.

16. A method of treating cancer, the method comprising:

a) preparing an immunogenic composition comprising a tumor cell lysate, an oxidized lipid, and a toll-like receptor 4 (TLR4) agonist, wherein the tumor cell lysate is or has been prepared from a sample of a tumor obtained from the mammalian subject with cancer;

b) administering to the subject an effective amount of the immunogenic composition.

17. A method of treating a mammalian subject with cancer, the method comprising:

a) preparing an immunogenic composition comprising a tumor cell lysate, an oxidized lipid, and a toll-like receptor 4 (TLR4) agonist, wherein the tumor cell lysate is or has been prepared from a sample of a tumor obtained from the mammalian subject with cancer;

b) administering to the subject an effective amount of the immunogenic composition.

18. The method of any one of embodiments 10-17, wherein the oxidized lipid comprises at least one phospholipid of oxidized 1-palmitoyl-2-arachidonyl-sn-glycero-3-phosphorylcholine (oxPAPC).

19. The method of embodiment 18, wherein the at least one phospholipid comprises at least one of the group consisting of POVPC, PGPC, PECPC, and PEIPC, optionally wherein the at least one phospholipid comprises PGPC.

20. The method of any one of embodiments 10-17, wherein the TLR4 agonist comprises monophosphoryl lipid A (MPLA).

21. The method of embodiment 20, wherein the TLR4 agonist is present within an adjuvant, and the adjuvant further comprises one or more of an aluminum salt, a saponin, and liposomes, optionally wherein the adjuvant is AS01B or AS04.

22. The method of embodiment any one of embodiments 10-17, wherein the least one phospholipid comprises PGPC, and the TLR4 agonist comprises monophosphoryl lipid A (MPLA).

23. The method of any one of claims 10-22, further comprising administering to the subject and effective amount of an additional therapeutic agent.

24. The method of embodiment 23, wherein the additional therapeutic agent comprises one or more of the group consisting of an immune checkpoint inhibitor, an antineoplastic agent, and radiation therapy.

25. The method of any one of embodiments 10-23, wherein the cancer was resistant to an immune checkpoint inhibitor prior to administration of the immunogenic composition.

### EXAMPLES

### Example 1: Hyperactive Dendritic Cells Simulate Durable Anti-Tumor Immunity to Complex Antigen Mixtures

The ideal strategy of stimulating protective immunity would be to combine the benefits of activated and pyroptotic DCs, whereby activated cells would have the ability to release IL-1β while maintaining viability. The inventors have recently identified a new activation state of DCs which display these attributes. When DCs are exposed to PAMPs (e.g. TLR ligands) and a collection of oxidized phospholipids released from dying cells (DAMPs), the cells achieve a long-lived state of “hyperactivation” (I. Zanoni, et al. Science, vol. 352, no. 6290, pp. 1232-1236, 2016; I. Zanoni, et al. Immunity, vol. 47, no. 4, p. 697-709.e3, 2017). The collection of oxidized lipids are known as oxPAPC (oxidized 1-palmitoyl-2-arachidonyl-sn-glycero-3-phosphorylcholine). Hyperactive DCs display the activities of activated DCs, in terms of cytokine release (e.g. TNFα), but they have gained the ability to also release IL-1β over the course of several days. Consistent with their assignment as “hyperactive” DCs, these cells are superior to their activated counterparts, in terms of their ability to stimulate T cell responses to model antigens.

Mechanisms underlying the hyperactive state of DCs have been defined, as the DAMPs in question (oxPAPC) are able to bind and stimulate the cytosolic PRR caspase-11 (I. Zanoni, et al. 2016). Caspase-11 stimulation results in the activation of NLRP3 and the assembly of an inflammasome that does not lead to pyroptosis, but rather leads to the release of IL-1β from living cells. IL-1β release from hyperactive cells is mediated by the pore forming protein gasdermin D, which serves as a conduit for the secretion of these cytokines (C. L. Evavold, et al. Immunity, 2018 Jan. 16; 48(1):35-44.e6; X. Liu, et al. Nature, vol. 535, no. 7610, pp. 153-158, 2016; R. A. Aglietti, et al. Proc. Natl. Acad. Sci. U.S.A, vol. 113, no. 28, pp. 7858-63, July 2016; N. Kayagaki, et al. Nature, vol. 526, no. 7575, pp. 666-671, September 2015). It is thought that the plasma membrane is repaired to remove gasdermin D pores in a fashion that ensures cell viability (S. Rühl, et al. Science, vol. 362, no. 6417, pp. 956-960, November 2018), whereas in other instances (alum stimulation) membrane repair pathways may be overwhelmed and pyroptosis ensues. Despite this insight into the mechanisms of how IL-1β can be released from living cells, the physiological benefits of the hyperactive cell state in terms of instruction of adaptive immunity have been poorly defined.

### Materials and Methods

Mouse strains, and Tumor cell lines: C57BL/6J (Jax 000664), caspase-1/-11 dKO mice (Jax 016621), NLRP3KO (Jax 021302), Casp11KO (Jax 024698), OT-I (Jax 003831) and OT-II (Jax 004194) and BALB/c (Jax 000651) mice were purchased from Jackson Labs. For syngeneic tumor models in C57BL/6J, two melanoma cell lines were used. The parental cell line: B16.F10 and an OVA expressing cell line: B16.F100VA. For a syngeneic colorectal model, MC-38 cell line expressing OVA derived from C57BL6 murine colon adenocarcinoma cells was used. These cell lines were a gift from Arlene Sharpe Laboratory. For a syngeneic colon cancer model in BALB/c mice, CT26 cell line was used (a gift from Jeff Karp laboratory).

Reagents: E. coli LPS (Serotype 055:B5-TLRGRADE™) was purchased from Enzo and used at 1 μg/ml in cell culture or 10 μg/mice for in vivo use. Monophosphoryl Lipid A from S. minnesota R595 (MPLA) was purchased from Invivogen and used at 1 μg/ml in cell culture or 20 μg/mice for in vivo use. OxPAPC was purchased from Invivogen, resuspended in pre-warmed serum-free media and was used as 100 μg/ml for cell stimulation, or 65 μg/mice for in vivo use. POVPC and PGPC were purchased from Cayman Chemical. Reconstitution of commercially available POVPC and PGPC was performed as previously described (C. L. Evavold et al., Immunity, 2018 Jan. 16; 48(1):35-44). Briefly, ethanol solvent is evaporated using a gentle nitrogen gas stream. Pre-warmed serum-free media was then immediately added to the dried lipids to a final concentrations of 1 mg/ml. Reconstituted lipids were incubated at 37° C. for 5-10 mins and were sonicated for 20 s before adding to cells. POVPC or PGPC were used at 100 μg/ml for cell stimulation or 65 μg/mice for in vivo use. EndoFit chicken egg ovalbumin protein with endotoxin levels <1 EU/mg and OVA 257-264 peptide were purchased from Invivogen for in vivo use at a concentration of 200 μg/mice or in vitro use at a concentration of 500 or 100 μg/ml. Incomplete Freund's Adjuvant (F5506) was purchased from Sigma and used for in vivo immunizations at a working concentration of 1:4 (IFA:antigen emulsion). Alhydrogel referred to as alum was purchased from Accurate Chemical, and used for in vivo immunization at a working concentration of 2 mg/mouse. In some experiments, Addavax which is a Squalene-oil-in-water adjuvant was used instead of IFA at a working concentration of 1:2 (AddaVax: antigen).

Cell culture: BMDCs were generated by differentiating bone marrow in IMDM (Gibco), 10% B16-GM-CSF derived supernatant, 2 μM 2-mercaptoethanol, 100 U/ml penicillin, 100 μg/ml streptomycin (Sigma-Aldrich) and 10% FBS. 6 day after culture, BMDCs were washed with PBS and re-plated in IMDM with 10% FBS at a concentration of 1×10⁶ cells/ml in a final volume of 100 μl. CD11c⁺ DC purity was assessed by flow cytometry using BD Fortessa and was routinely above 80%. Splenic DCs from mice injected with B16-FLT3 for 15 days, were purified as CD11c⁺MHC⁺ live cells, then plated at a concentration of 1×10⁶ cells/ml in a final volume of 100 μl in complete IMDM. To induce hyperactive or pyroptotic BMDCs, DCs were primed with LPS (1 μg/ml) for 3 hours, then stimulated with OxPAPC or PGPC (100 μg/ml) or alum (100 μg/ml) for 21 h in complete IMDM. In some cases, activated BMDCs were re-stimulated for additional 24 h onto plate-bound agonistic anti-CD40, using Ultra-LEAF anti-mouse CD40 (clone 1C10; BioLegend). T cells were cultured in RPMI-1640 (Gibco) supplemented with 10% FBS, 100 U/ml penicillin, 100 μg/ml streptomycin (Sigma-Aldrich), and 50 μM β-mercaptoethanol (Sigma-Aldrich). Tumor cell lines were all cultured in DMEM supplemented with 10% FBS. For OVA expressing cell lines, puromycin (2 μg/ml) was added to the media.

LDH Assay and ELISAs: Fresh supernatants were clarified by centrifugation after BMDC stimulation, then assayed for LDH release assay using the Pierce LDH cytotoxicity colorimetric assay kit (Life Technologies) following the manufacturer's protocol. Measurements for absorbance readings were performed on a Tecan plate reader at wavelengths of 490 nm and 680 nm. To measure secreted cytokines, supernatants were collected, clarified by centrifugation and stored at −20° C. ELISA for IL-1β, TNFα, IL-10, IL-12p70, IFNγ, IL-2, IL-13, IL-4 and IL-17 were performed using eBioscience Ready-SET-Go! (ThermoFisher) ELISA kits according to the manufacturer's protocol.

Flow cytometry: After FcR blockade, 7 days BMDCs were resuspended in MACS buffer (PBS with 1% FCS and 2 mM EDTA), and stained with the following fluorescently conjugated antibodies (BioLegend): anti-CD11c (clone N418), anti-I-A/I-E (clone M5/114.15.2), anti-CD40 (clone 3/23), anti-CD80(16-10A1), anti-CD69 clone (H1.2F3), anti-H-2Kb (clone AF6-88.5). Single cell suspension from the tumor or draining inguinal lymph nodes, or skin inguinal adipose tissue were resuspended in MACS buffer (PBS with 1% FCS and 2 mM EDTA), and stained with the following fluorescently conjugated antibodies (BioLegend): anti-CD8α (clone 53-6.7), anti-CD4 (clone RM4-5), anti-CD44 (clone IM7), anti-CD62L (MEL-14), anti-CD3 (17A2), anti-CD103 (2E7), anti-CD69 clone (H1.2F3), anti-CD45 (A20 or 30F11). LIVE/DEAD™ Fixable Violet Dead Cell Stain Kit (Molecular probes) was used to determine the viability of cells, and cells were stained for 20 minutes in PBS at 4° C. Draining inguinal lymph nodes T cells were stained with OVA-peptide tetramers at room temperature for 1 h. PE-conjugated H2K(b) SIINFEKL (OVA 257-264; SEQ ID NO: 1) and APC conjugated I-A(b) AAHAEINEA (OVA 329-337; SEQ ID NO: 2) were used. I-A(b) and H2K(b) associated with CLIP peptides were used as isotype controls. Tetramers were purchased for NIH tetramer core facility. in some experiments, FITC anti-CD8.1 (clone Lyt-2.1 CD8-E1) purchased from accurate chemical was used with tetramers. To determine absolute number of cells, COUNTBRIGHT counting beads (Molecular probes) were used, following the manufacturer's protocol. Appropriate isotype controls were used as a staining control. Data were acquired on a BD FACS ARIA or BD Fortessa. Data were analyzed using FlowJo software.

Antigen uptake assay: To examine antigen uptake and the endocytic ability of BMDC during different activation states (active, hyperactive or pyroptotic states), FITC labeled-chicken OVA (FITC-OVA) was used (Invitrogen-Molecular Probes). Briefly, pretreated BMDCs were incubated with either FITC-OVA or AF488-dextran (0.5 mg/ml) during 45 minutes at 37° C., or 4° C. (as a control for surface binding of the antigen). BMDCs were then washed, and stained with Live/Dead Fixable Violet Dead Cell Stain Kit (Molecular probes) to distinguish living cells from dead cells. Cells were then fixed with BD fixation solution and resuspended in MACS buffer (PBS with 1% FCS and 2 mM EDTA). FITC fluorescence of live cells was measured every 15 minutes using Fortessa flow cytometer (Becton-Dickenson). Fluorescence values of BMDCs incubated at 37° C. were reported as percentage of OVA-FITC or Dextran-AF488 associated cells and data were normalized to the percentage of OVA-FITC associated cells incubated at 4° C.

OVA antigen presentation assay: To measure the efficiency of OVA antigen presentation on MHC-I, (0.5×10⁶) BMDCs treated with an activation (LPS), a hyperactivation (LPS+PGPC or LPS+OxPAPC) or a pyroptotic stimuli (LPS+Alum) were incubated with Endofit-OVA protein (0.5 mg/ml) for 2 hours at 37° C. Cells were then washed with MACS buffer and stained on ice for 20 to 30 min with APC anti-mouse H-2K^{b} antibody (Clone AF6-88.5, BioLegend), and PE-conjugated antibody that binds to H-2K^{b} bound to the OVA peptide SIINFEKL (SEQ ID NO: 1; Clone 25-D1.16, BioLegend). Appropriate isotype controls were used as a staining control. The percentage of total surface H-2K^{b}, and the percentage of cells associated with the OVA peptide on MHC-I was calculated. Data were acquired on a Fortessa flow cytometer (Becton-Dickenson) and analyzed with FlowJo software (Tree Star).

OT-I and OT-II in vitro T-cell stimulation: Splenic CD8⁺ and CD4⁺ T cells were sorted from OT-I and OT-II mice by magnetic cell sorting with anti-CD8 beads or anti-CD4 beads respectively (Miltenyi Biotech). Sorted T cells were then seeded in 96-well plates at a concentration of 100.000 cells per well in the presence of either 20.000 or 10.000 DCs (5:1 or 10:1 ratio) that were pretreated with either LPS (activation stimuli) or LPS+PGPC (hyperactivation stimuli) or LPS+Alum (pyroptotic stimuli) and pulsed (or not) with OVA protein or SIINFEKL (SEQ ID NO: 1) peptide at 100 μg/ml for 2 hours. 5 days post culture, supernatants were collected and clarified by centrifugation for short-term storage at −20° C. and cytokine measurement by ELISA.

Intracellular staining: For intracellular cytokine staining, cells were stimulated with 50 ng/ml phorbol 12-myristate 13-acetate (PMA) and 500 ng/ml ionomycin (Sigma-Aldrich) in the presence of GolgiStop (BD) and brefeldin A for 4-5 h. Cells were then washed twice with PBS, and stained with LIVE/DEAD™ Fixable violet or green Dead Cell Stain Kit (Molecular probes) in PBS for 20 min at 4° C. Cells were washed with MACS buffer, and stained for appropriate surface markers for 20 min at 4° C. After two washes, cells were fixed and permeabilized using BD Cytofix/Cytoperm kit for 20 min at 4° C., then washed with 1× perm wash buffer (BD) per manufacturer's protocol. Intracellular cytokine staining was performed in 1× perm buffer for 20-30 min at 4° C. with the following conjugated antibodies all purchased from BioLegend: anti-Ki67 (clone 16A8), anti-IFN-γ (clone XMG1.2), anti TNFα (clone MP6-XT22), anti-Gata3 (16E10A23), anti-IL4(11B11), anti-IL10 (clone JES5-16E3). Data were acquired on a BD FACS ARIA or BD Fortessa. Data were analyzed using FlowJo software.

In vivo immunization and T cell re-stimulation: Female C57BL/6J mice aged 8 weeks, were immunized subcutaneously (s.c.) on the left lower back with either 150 μg/mouse endotoxin-free OVA plus 10 μg/mouse LPS emulsified in incomplete Freund's adjuvant, or with 150 μg/mouse endotoxin-free OVA, plus 65 μg/mouse oxPAPC or PGPC, plus 10 μg/mouse LPS emulsified in incomplete Freund's adjuvant. In some experiments mice were injected s.c. with OVA alone or with LPS emulsified in Alum. 7 or 40 days after immunization, CD4⁺ T cells and CD8⁺ T cells were isolated from the draining lymph nodes of immunized mice by magnetic cell sorting with anti-CD4 beads or anti-CD8 beads and columns (Miltenyi Biotech). Enriched cells were then sorted as live CD45⁺CD3⁺CD4⁺ cells or live CD45⁺CD3⁺CD8⁺ using FACS ARIA. Purity post-sorting was >98%. Sorted cells were then seeded in 96-well plates at a concentration of 100.000 cells per well in the presence of 10-20×10³ DC pulsed with serial dilutions of OVA, starting at 1 mg/ml. Secretion of IFNγ, IL-10 and IL-2 were measured by ELISA 5 days later.

CD107a Degranulation Assay: To evaluate the effector antitumor activity of CD8⁺ T cells, surface exposure of the lysosomal-associated protein CD107a was assessed by flow cytometry. Briefly, CD8⁺ T cells from the skin draining lymph nodes of immunized mice were isolated by magnetic cell enrichment with anti-CD8 beads and columns (Miltenyi Biotech), then sorted as CD3⁺CD8⁺ Live cells on FACS ARIA(BD). Freshly sorted CD8⁺ T cells were resuspended in complete RPMI at a concentration of 1×10⁶ cells/ml. PerCP/Cy5.5 anti-mouse CD107a (LAMP-1) antibody (Clone1D4B, BioLegend) was added at a concentration of 1 μg/ml to this media, in the presence of GolgiStop (BD). T cells were then immediately seeded as 100,000 cells onto 10,000 MC380VA or B160VA tumor cells/well in 96 wells plates. Alternatively, CD8⁺ T cells were seeded alone and stimulated with 50 ng/ml phorbol 12-myristate 13-acetate (PMA) and 500 ng/ml ionomycin (Sigma-Aldrich). 5 hours post-culture, cells were washed with MACS buffer, stained with LIVE/DEAD™ Fixable Violet Dead Cell Stain Kit (Molecular probes), and APC anti-CD8 (Clone 53-6.7, BioLegend). Cells were then Fixed with BD fixation solution for 20 min at 4° C. and resuspended in MACS buffer. The percentage of CD107a+ cells was determined by flow cytometry on the Fortessa flow cytometer (BD).

In vitro cytotoxicity assay: CD8⁺ T cells from the spleen, or the skin inguinal adipose tissue of survivor mice were isolated using anti-CD8 MACS beads and columns (Miltenyi Biotec). Enriched T cells were then sorted as live CD45⁺CD3⁺CD8⁺ cells using FACS ARIA. Purity post-sorting was >97%. Tumor cell lines such as B16OVA, B16F-10 or CT26 cells were seeded onto 96-well plates (2×10⁴ cells/well) in complete DMEM at least 5 hours prior their co-culture with T cells. 10⁵ CD8⁺ T cells were seeded onto tumor cells for 12 h, then cytotoxicity was assessed by LDH release assay using the Pierce LDH cytotoxicity colorimetric assay kit (Life Technologies) following the manufacturer's protocol.

Whole tumor cell lysates preparation: To prepare whole tumor cell lysates (WTL) for immunization, tumor cell lines were cultured for 4-5 days in complete DMEM. When cells became confluent, supernatants were collected, and the cells were washed and dissociated using trypsin-EDTA (Gibco). Tumor cell lines were then resuspended at 5×10⁶ cells/ml in their collected culture supernatant, then lysed by 3 cycles of freeze-thawing.

Syngeneic whole tumor lysates of melanoma or colon adenocarcinoma tumors were prepared from explanted tumors of unimmunized tumor-bearing mice. Briefly, tumors were mechanically disaggregated using gentleMACS dissociator (Miltenyi Biotec), then heated at 42° C. in a water bath for 15 minutes and then digested using the tumor Dissociation Kit (Miltenyi Biotec) following the manufacturer's protocol. After digestion, tumors were washed with PBS and passed through 70-μm and 30-μm filters, then depleted of CD45⁺ cells using CD45 microbeads (Miltenyi Biotec). Tumor cells were resuspended at 5×10⁶ cells/ml then lysed by 3 cycles of freeze-thawing. WTL preparations were centrifuged at 12.000 rpm for 15 minutes, passed through 70-μm and 30-μm filters then stored in aliquots at −20° C. until use. WTL were used for immunizations, immunotherapy or DC as described in the following section at a concentration equivalent to 2.5×10⁵ tumor cells per mice.

In vivo immunization and tumor challenges: For immunization prior tumor inoculation, C57BL/6 mice were injected subcutaneously (s.c.) into the right flank with PBS (unimmunized), WTL alone or with LPS, or WTL plus LPS and OxPAPC or PGPC all emulsified in incomplete Freud's adjuvant (IFA). In some experiments, LPS is replaced by MPLA. 15 days post immunization, mice were challenged s.c. on the left flank with 3×10⁵ of viable B160VA cells, or 3×10⁵ B16-F10 cells, or 5×10⁵ of viable MC38-OVA cells as indicated. Tumor-free mice were re-challenged s.c. into the upper back with a lethal dose of 5×10⁵ viable B16OVA or B16F-10 cells, or 1×10⁶ of viable MC38-OVA cells as indicated. In some experiments, mice were given 100 μg of LEAF anti-mouse/rat IL-1β antibody (BioLegend) by i.v. injection two days and one day before receiving the immunization. Antibody treatment continued 1, 2, and 3 days after immunization to ensure chronic depletion of circulating IL-1β.

For immunizations in the context of an immunotherapeutic approach, C57BL/6J were injected on the left flank with 3×10⁵ of viable B160VA cells, or 3×10⁵ of B16-F10 cells, or 5×10⁵ of viable MC38-OVA cells. Alternatively, BALB/c mice were injected on the left flank with 5×10⁵ of viable CT26 cells. Where indicated after tumor inoculation, mice were either left untreated (unimmunized) or immunized with WTL plus LPS and PGPC emulsified in incomplete Freud's adjuvant (IFA). Immunizations were followed by two boost injections as indicated in the immunizations schedule located on top of each survival graph. Where indicated, mice were given 100 μg of antibody intraperitoneally (i.p.) during the same days of immunization or boost injection, then every other 3 days for a total of 4 injections, using the following antibodies: and anti-PD-1 (clone 29F.1A12), Ultra-LEAF anti-CD4 (clone GK1.5), and anti-CD8a (clone 53-6.7). Alternatively, mice were given 100 ug of LEAF anti-mouse/rat IL-1β antibody by i.v. injection two days and one day before receiving the immunization/boost. Anti-IL-1β treatment continued as previously mentioned 1, 2, and 3 days after immunization to ensure chronic depletion of circulating IL-1. Control mice received isotype-matched rat IgG. All antibodies were purchased from BioLegend.

The size of the tumors was assessed in a blinded, coded fashion every two days and recorded as tumor area (length×width) with a caliper. Mice were sacrificed when tumors reached 2 cm³ or upon ulceration.

In vivo Immunization and B16-F10 pulmonary colonization: For inducing experimental lung colonization, 3×10⁵ B16-F10 tumor cells were injected i.v. via tail vein in a volume of 100 ul. 2 days before tumor inoculation, mice were either left untreated (unimmunized) or were immunized s.c. on the right flank with WTL alone or with LPS, or with WTL plus LPS and PGPC all emulsified in Addavax. Mice received a boost injection 5 days post tumor inoculation. Mice were then sacrificed on day 18 after tumor injection, and lung tissues were isolated and fixed in Fekete's solution. Lung metastatic nodules present on the surface of the lungs per mouse were counted.

Tumor Infiltration: To assess the frequency of tumor-infiltrating lymphocytes (TIL) in immunized mice, tumors were harvested when their size reached 1.8-2 cm. Tumors were dissociated using the tumor Dissociation Kit (Milteny Biotec) and the gentleMACS dissociator following the manufacturer's protocol. After digestion, tumors were washed with PBS and passed through 70-μm and 30-μm filters. CD45⁺ cells were positively selected using CD45 microbeads (Milteny Biotec), and T cell infiltration was assessed by flow cytometry. Tumor infiltrating T cells were cultured with dynabeads mouse T-Activator CD3/CD28 (Gibco) for T cell activation and expansion.

Adoptive cell transfer: For T cell transfer, CD8⁺ T cells from the spleen, or the skin inguinal adipose tissue of survivor mice were isolated using anti-CD8 MACS beads and columns (Milteny Biotec). Enriched cells were then sorted as live CD45⁺CD3⁺CD8⁺ cells using FACS ARIA. Purity post-sorting was >97%. Sorted T cells were then stimulated for 24 h in 24-well plates (˜2×10⁶ cells/well) coated with anti-CD3 (4 μg/ml) and anti-CD28 (4 μg/ml) in the presence of IL-2 (50 ng/ml). 5×10⁵ of activated circulating splenic or skin inguinal adipose resident CD8⁺ T cells were transferred by i.v. or intra dermal (i.d.) injection respectively into naïve recipient mice. Some mice received both T cell subsets.

Statistical analysis: Statistical significance for experiments with more than two groups was tested with two-way ANOVA with Tukey multiple comparison test correction. Adjusted p-values, calculated with Prism (Graphpad), are coded by asterisks: <0.05 (*); <0.0005 (***); <0.0001 (****).

### Results

Hyperactivating Stimuli Upregulate Several Activities Important for DCs to Stimulate T Cell Immunity.

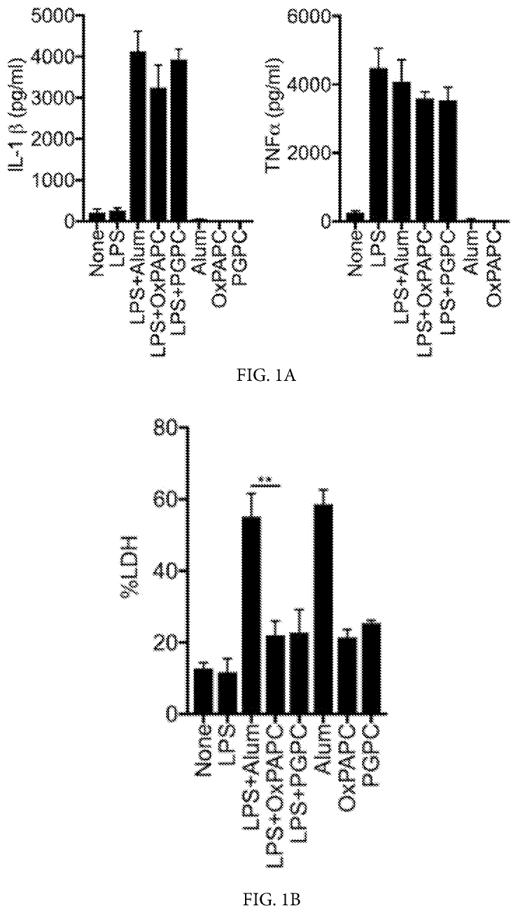

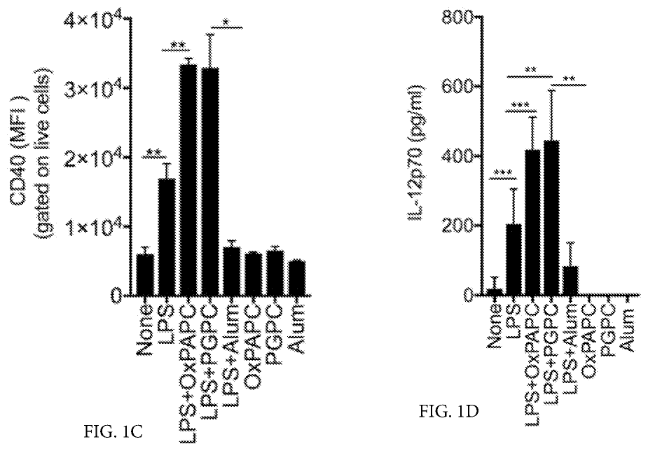

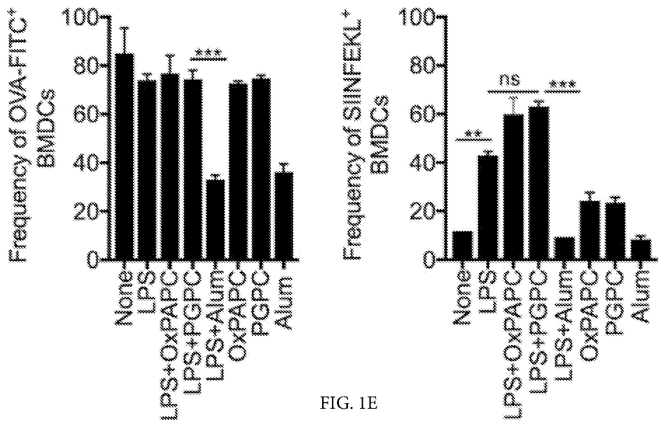

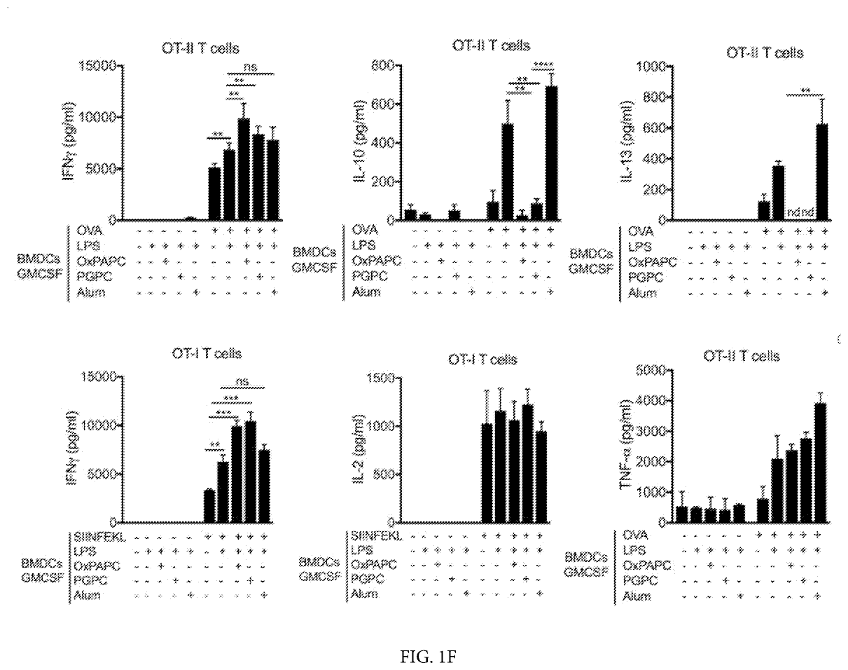

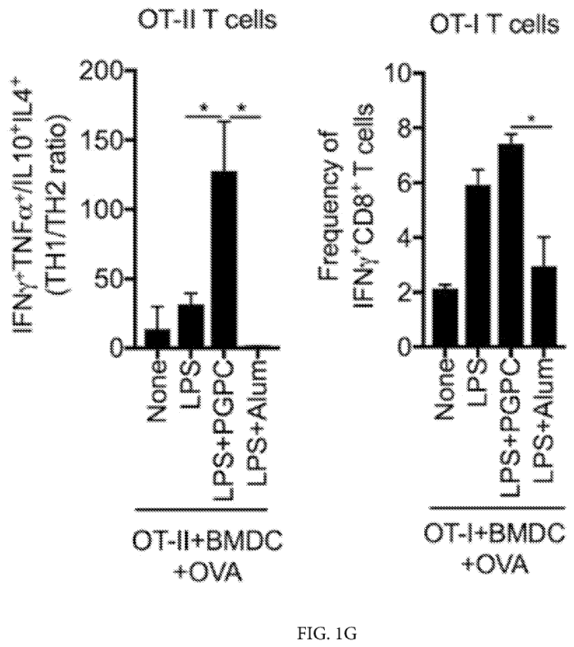

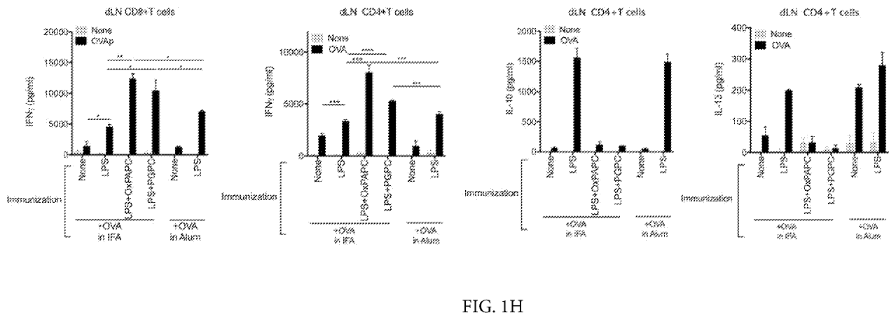

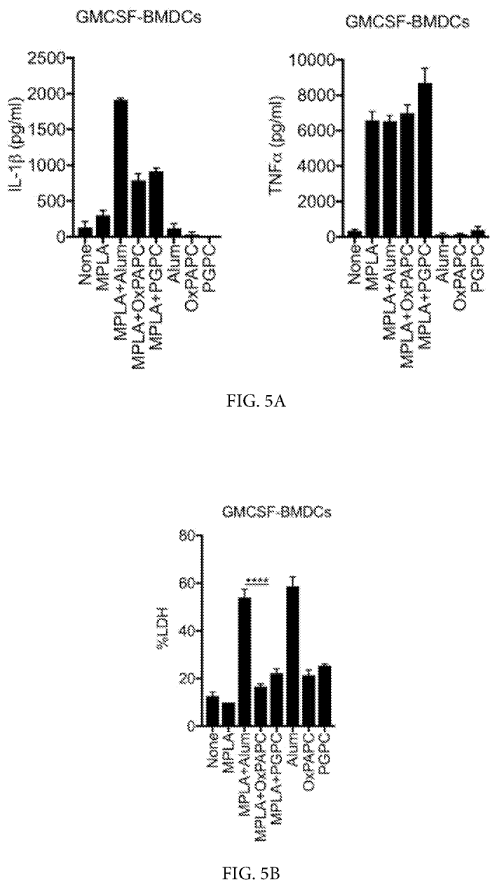

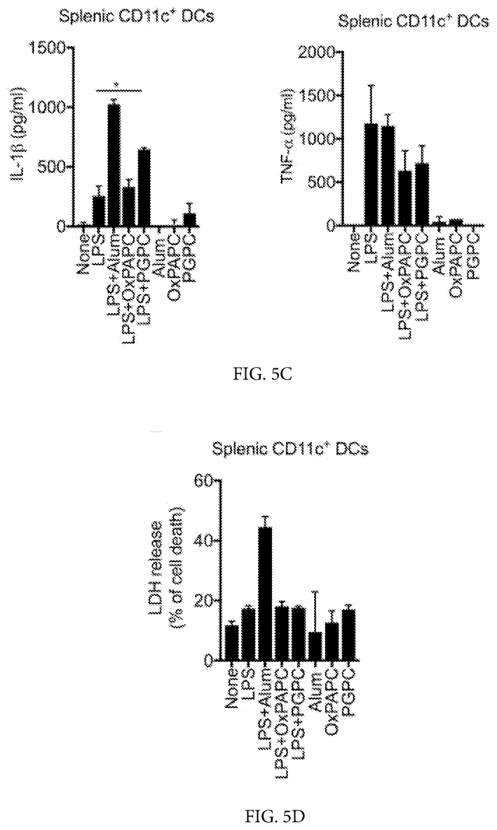

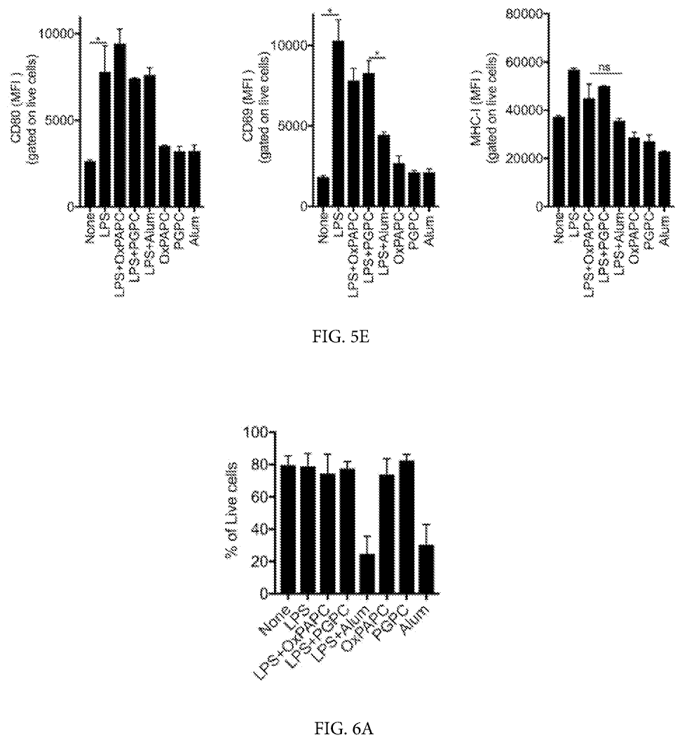

Virtually all studies of DC hyperactivation have focused on the ability of these cells to release IL-1β while retaining viability. The spectrum of DC functions that are influenced by hyperactivating stimuli are undefined. To examine this spectrum, bone marrow derived DC (BMDCs) were primed with LPS and subsequently treated with oxPAPC or a specific and pure lipid component of oxPAPC named PGPC (I. Zanoni, et al. Science, vol. 352, no. 6290, pp. 1232-1236, 2016). The resulting hyperactive cells were compared to traditionally activated BMDCs (treated with LPS) or pyroptotic BMDCs (primed with LPS and subsequently treated with alum). In contrast to activation stimuli, which expectedly did not induce the release of IL-1β, pyroptotic or hyperactivating stimuli promoted IL-1β release into the extracellular media (FIG. 1A). All stimuli examined promoted the secretion of the cytokine TNFα (FIG. 1A). These findings are in accordance with prior work, which established that LPS-activated BMDCs release TNFα but not IL-1β (I. Zanoni et al., 2016. IL-1β secretion was co-incident with cell death in pyroptotic DCs, as assessed by the release of the cytosolic enzyme lactate dehydrogenase (LDH) (FIG. 1B). In contrast, IL-1β secretion occurred in the absence of LDH release in hyperactive cells (FIG. 1B). Similar behaviors of BMDCs were observed when LPS was replaced with MPLA (FIGS. 5A, 5B), an FDA-approved TLR4 ligand that is used in vaccines against human papilloma virus (HPV) and hepatitis B virus (HBV). To determine if the behavior of hyperactive BMDCs extends to DCs that were differentiated in vivo, activities of CD11c⁺ DCs isolated from the spleen of mice that were injected with B16-FLT3, were examined. Similar to the behavior of GMCSF-derived BMDCs, treatment with LPS and PGPC led to the release of TNFα and IL-1β from splenic CD11c⁺ DCs in the absence of cell death, as assessed by LDH release (FIGS. 5C, 5D). These results indicate that the hyperactivating stimulus PGPC can be used to induce IL-1β release from living DCs that have been differentiated in vitro or in vivo.

Several signals important for T cell differentiation, such as the expression of the co-stimulatory molecules CD80, CD69 and CD40, and the secretion of the p70 subunit of IL-12, were examined. CD80 surface expression was similar in DCs responding to all activation stimuli (FIG. 5E). In contrast, CD40 expression was highly influenced by activation stimulus. As compared to the activating stimulus LPS, hyperactivating stimuli induced greater expression of CD40 (FIG. 1C). Pyroptotic stimuli were very weak inducers of CD40 and CD69, even within the 20-30% of living cells that remained after LPS-alum treatments (FIGS. 1C and 5E). The differential expression of CD40 correlated with hyperactive DCs having the greatest ability to secrete IL-12p70 when cultured onto agonistic anti-CD40 coated plates (FIG. 1D).

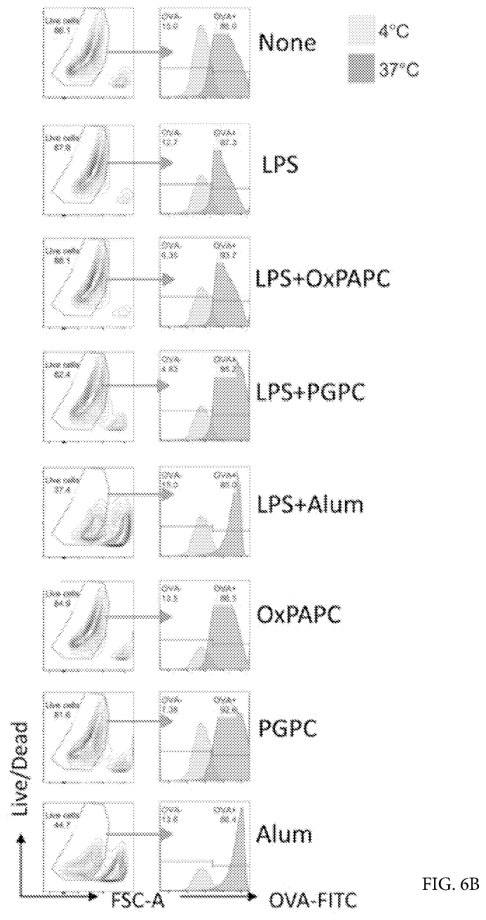

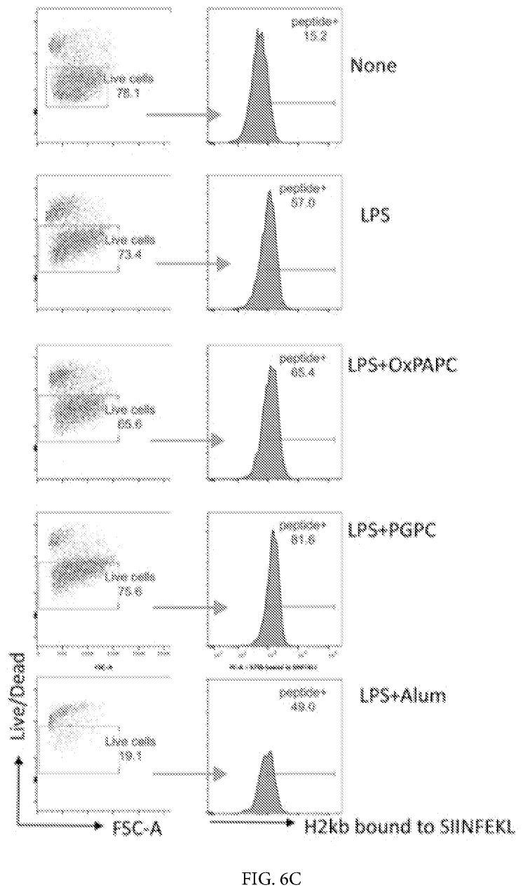

Hyperactive BMDCs were no better than their activated counterparts at antigen capture, as assessed by the equivalent internalization of fluorescent ovalbumin (OVA-FITC) (FIGS. 6A, 6B), yet the former cell population displayed a greater abundance of OVA-derived SIINFEKL peptide on MHC-I molecules at the cell surface (FIGS. 1E and 6C). Total surface MHC-I abundance did not differ between activated and hyperactivated cells (FIG. 5E). Taken together, as compared to other stimuli of DCs, hyperactivating stimuli exhibit an enhancement of several activities important for T cell differentiation.

Hyperactive DCs Stimulate a TH1-Focused Immune Response, with No Evidence of TH2 Immunity.

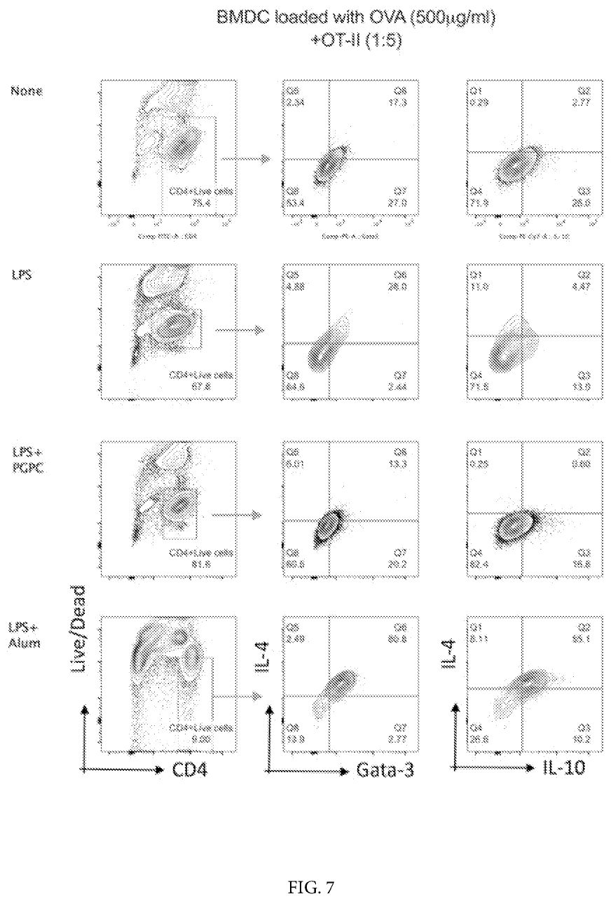

To assess the influence of DC activation states on T cell instruction, BMDCs were treated as described above, and were then loaded with OVA. These cells were exposed to naïve OT-II or OT-I T cells. OT-II cells express a T cell Receptor (TCR) specific for an MHC-II restricted OVA peptide (OVA 323-339), whereas OT-I cells express a TCR specific for an MHC-I restricted OVA peptide (OVA 257-264) (K. A. Hogquist, et al. Cell, vol. 76, no. 1, pp. 17-27, January 1994; M. J. Bamden, et al. Immunol. Cell Biol., vol. 76, no. 1, pp. 34-40, February 1998). The activities of the responding T cells were assessed by ELISA, to determine T cell polarization towards TH1 responses (IFNγ production), or TH2 responses (IL-10, IL-4 or IL-13 production). Regardless of DC activation state, OVA treated BMDCs stimulated the production of IFNγ from OT-II T cells. The extent of IFNγ production varied modestly between activation stimuli examined (FIG. 1F). Similarly, TNFα production by responding OT-II cells was comparable when comparing all DC activation states (FIG. 1F). These results indicate that in vitro, TH1 responses are commonly induced, regardless of the activation state of the antigen presenting cell (APC). In contrast, TH2 responses were strikingly different when comparing DC activation states. Stimuli that induce BMDC activation (LPS) or pyroptosis (LPS+alum) promoted the release of large amounts of IL-10, and IL-13, whereas hyperactivating stimuli led to minimal production of these TH2-associated cytokines (FIG. 1F). Intracellular staining of single cells for TH1 (IFNγ and TNFα) and TH2 (IL-4 and IL-10) cytokines as well as the TH2-lineage defining transcription factor GATA3 permitted the calculation of the ratio of TH1 and TH2 cells generated by different DC activating stimuli. This analysis revealed that hyperactive BMDCs induce a strong skewing of individual T cells towards the IFNγ-producing TH1 lineage (FIGS. 1G and 7). The ratio of TH1 to TH2 cells under hyperactive conditions was greater than 100:1 (FIG. 1G). In contrast, all other activating stimuli induced a mixed T cell response, with pyroptotic stimuli leading to a nearly 1:1 ratio of TH1 to TH2 cells (FIG. 1G).

Similar studies were done with CD8⁺ OT-I T cells, which revealed a slight enhancement of IFNγ production by stimuli that hyperactivate BMDCs, as compared to activating or pyroptotic stimuli (FIG. 1F). IL-2 production by responding OT-I cells was comparable when comparing all DC activation states (FIG. 1F). These collective results indicate that in vitro, the hyperactive BMDC state leads to a highly TH1-biased T cell response and a slightly enhanced CD8 T cell response. In contrast, activation or pyroptotic stimuli yielded a mixed TH1 and TH2 response.

To determine if the observations made in vitro with hyperactive BMDCs and OT-I and OT-II T cells apply to endogenous scenarios in vivo, the ability of hyperactivating stimuli to promote antigen-specific T cell responses in vivo, was examined. Mice were immunized with OVA either alone or with activating stimuli (LPS), or with hyperactivating stimuli (LPS+oxPAPC or PGPC) or with pyroptotic stimuli (LPS+alum, or alum alone). Forty days post-immunization, CD4⁺ or CD8⁺ T cells were re-stimulated ex vivo with naïve BMDCs that were loaded with OVA (or not). Immunizations with hyperactivating stimuli led to a greater amount of IFNγ production by responding T cells than immunizations with LPS or pyroptotic stimuli (LPS+alum) (FIG. 1H). Notably, no IL-10 or IL-4 or IL-13 was produced by T cells from mice immunized with hyperactivating stimuli (FIGS. 1H and 7). Thus, similar to the observations in vitro with transgenic T cells, hyperactivating stimuli were capable of inducing TH1-biased and CD8⁺ T cell responses from endogenous T cells in vivo.

In contrast to the findings with hyperactivating stimuli, activating stimuli (LPS) led to a mixed TH1 and TH2 phenotype, with T cells producing IFNγ, IL-10, IL-4 and IL-13 (FIGS. 1H and 7). Immunizations with OVA and alum alone led to a highly skewed IL-13 response, with no IFNγ being detected by responding T cells (FIG. 1H). These results are in accordance with previous studies showing that alum is particularly poor at promoting type I immunity (E. Oleszycka, et al. Eur. J. Immunol., vol. 48, no. 4, pp. 705-715, April 2018; M. J. Newman, et al. J. Immunol., vol. 148, no. 8, pp. 2357-62, April 1992), and that alum leads to TH2-biased immune responses (M. Kool, et al. J. Exp. Med., vol. 205, no. 4, pp. 869-882, April 2008; T. Marichal, et al. Nat. Med., vol. 17, no. 8, pp. 996-1002, August 2011).

One difference between LPS and LPS+oxPAPC is the ability of the latter to induce IL-1β secretion, as described above. Since alum and oxPAPC treatment lead to the NLRP3-dependent release of IL-1β (L. Franchi and G. Núñez, Eur. J. Immunol., vol. 38, no. 8, pp. 2085-9, August 2008; H. Li, et al. J. Immunol., vol. 178, no. 8, pp. 5271-6, April 2007), it was queried whether LPS+alum immunizations can phenocopy the T cell responses induced by LPS+oxPAPC or PGPC immunizations. The answer to this question was no; LPS+alum immunizations led to a balanced TH1:TH2 response, with ex vivo stimulated T cells producing ample amounts of IFNγ, IL-10, IL-4 and IL-13 (FIGS. 1H and 7). These results indicate that not all NLRP3 agonists stimulate a TH2-focused immune response.

Hyperactive Stimuli Enhance Memory T Cell Generation and Potentiates Antigen-Specific IFNγ Effector Responses in an NLRP3-Dependent Manner.

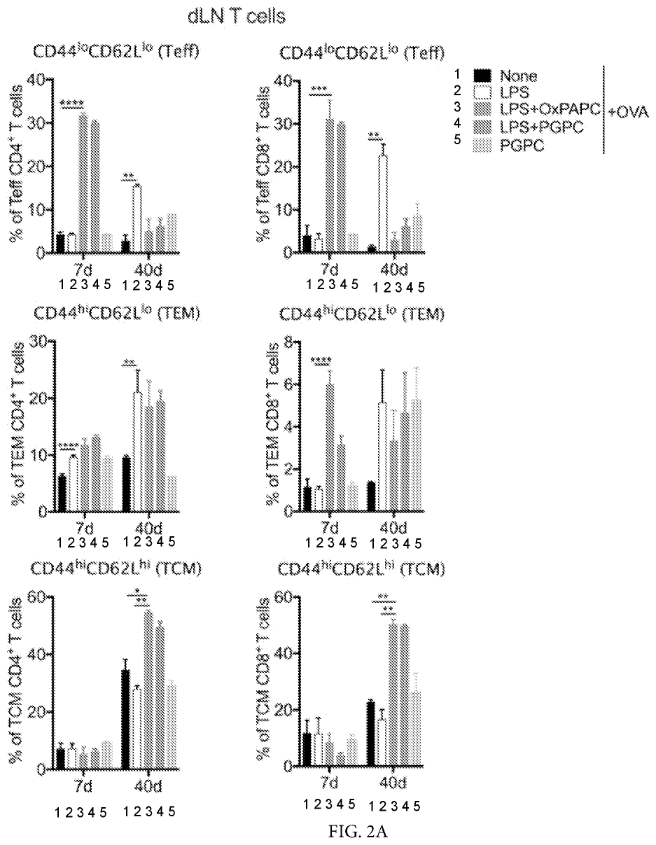

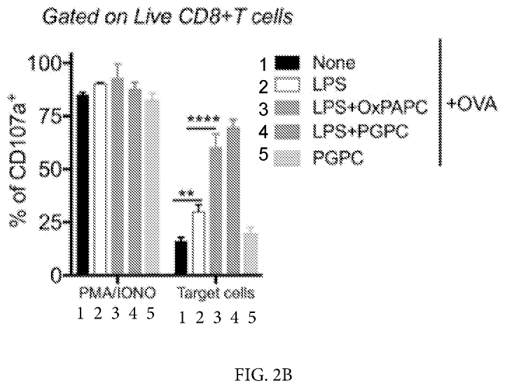

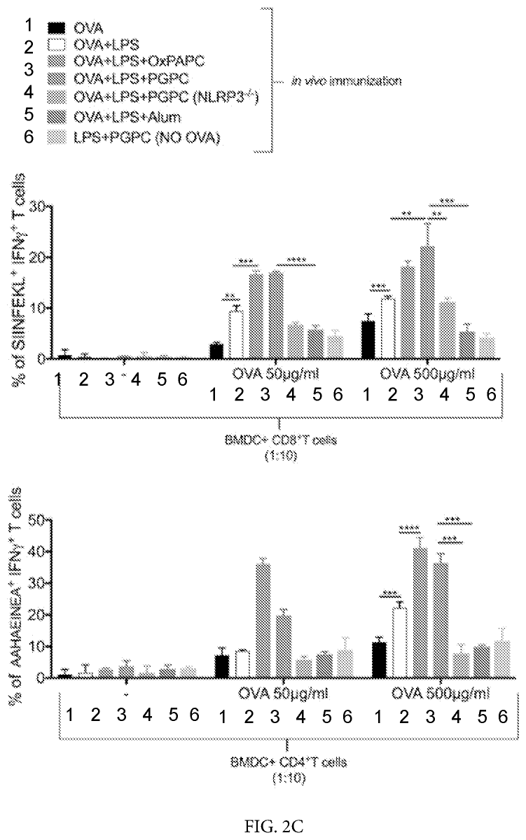

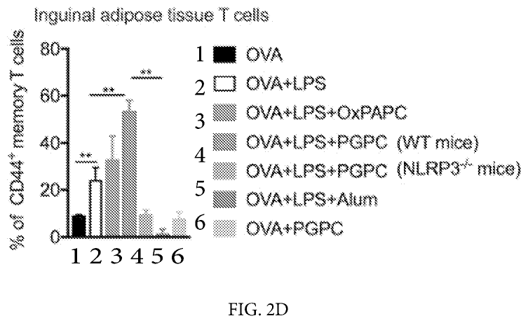

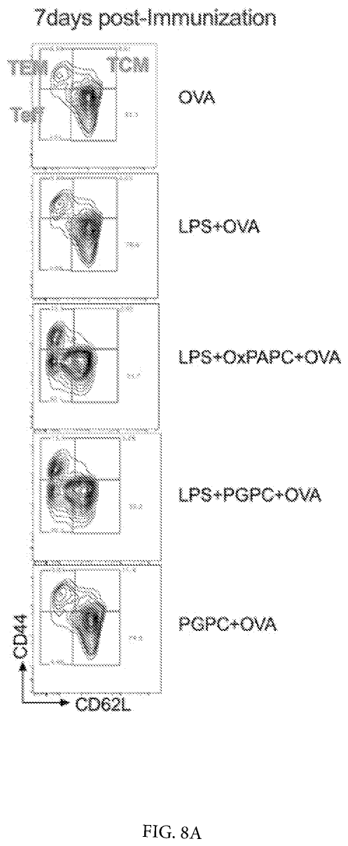

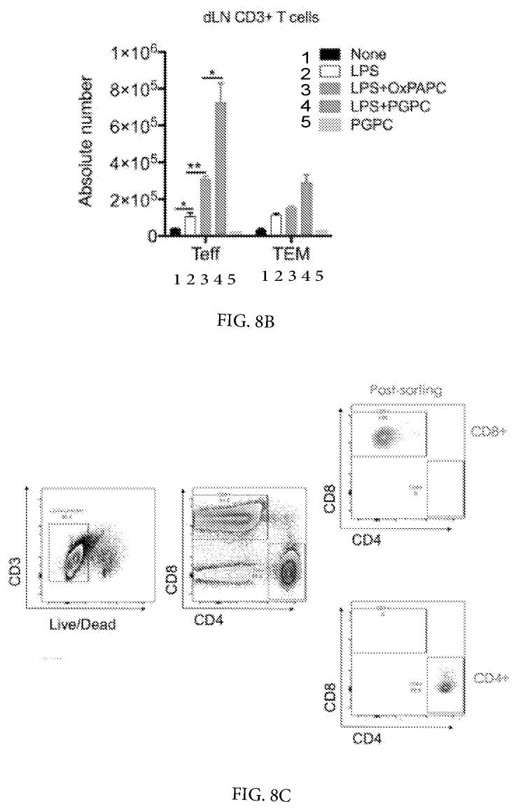

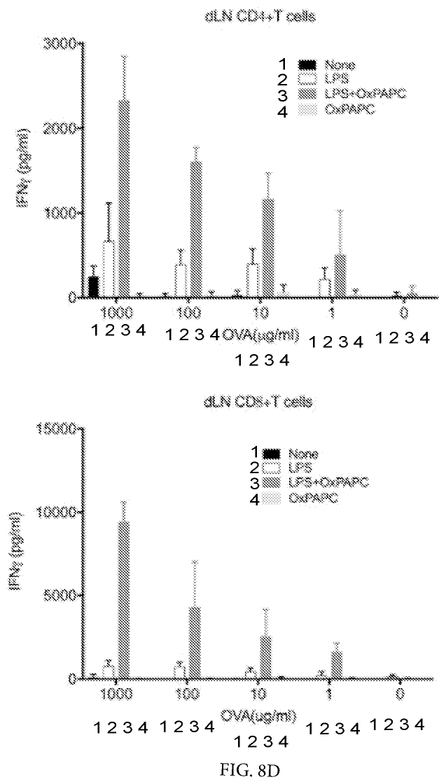

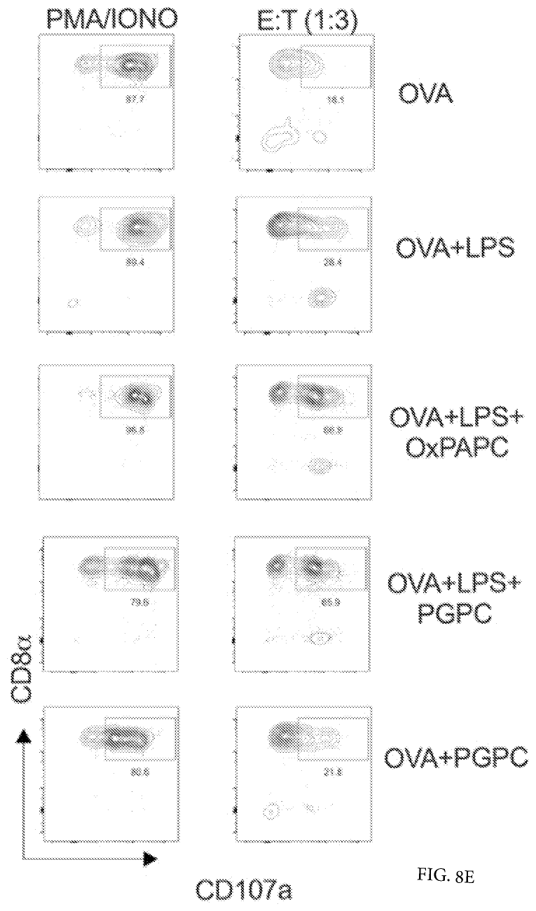

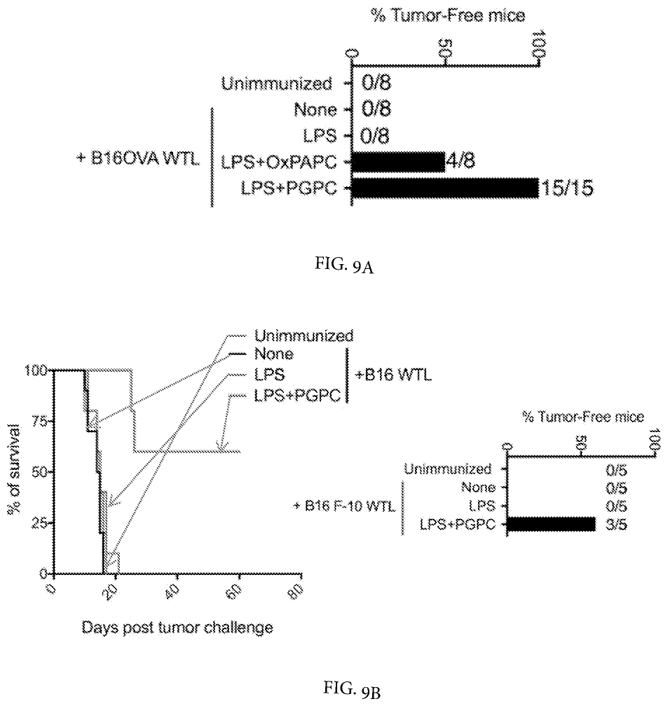

It was hypothesized that the enhanced T cell responses induced by hyperactivating stimuli could be explained by enhanced memory T cell generation and effector memory T cell responses. To examine this possibility, mice were immunized with OVA alone, or OVA plus an activating stimulus (LPS), or OVA plus a hyperactivating stimulus (LPS+oxPAPC or PGPC). 7- and 40-days post-immunization, memory and effector T cell generation in the dLN was assessed by flow cytometry using CD62L and CD44 markers that distinguish T effector cells (Teff) as CD44 ^{low}CD62L^{low}, T effector memory cells (TEM) as CD44^{hi}CD62L^{low}, and T central memory cells (TCM) as CD44^{hi}CD62L^{hi} (S. Z. Ben-Sasson, Cold Spring Harb. Symp. Quant. Biol., vol. 78, no. 0, pp. 117-124, January 2013). Seven days post-immunization, hyperactivating stimuli were superior than activating stimuli at inducing CD4⁺ and CD8⁺ Teff cells (FIGS. 2A upper panel and 8A, 8B). At this early time point, both stimuli induced low but comparable amounts of TEM cells (FIGS. 2A middle panel and 8A, 8B). Forty days post-immunization, ample TCM cells were observed in mice exposed to hyperactivating stimuli, whereas these cells were less abundant in mice immunized with OVA alone or with LPS (FIG. 2A lower panel). These data indicate that the hyperactivating stimuli oxPAPC and PGPC enhance the magnitude of effector and memory T cell generation. The increase in the frequency of Teff cells 7 days post-immunization correlated with the enhanced IFNγ responses of CD4⁺ and CD8⁺ T cells that were isolated from the dLN of immunized mice, and re-stimulated ex vivo in the presence of naïve BMDCs loaded with OVA (FIGS. 8C, 8D). Similarly, CD8⁺ T cell displayed enhanced degranulation ability, an activity associated with functional CTLs (FIG. 2B). When total CD8⁺ T cell were isolated from mice immunized with hyperactive stimuli and co-cultured with the B16 tumor cell line expressing OVA (B160VA), CD8⁺ T cells exhibited enhanced degranulation activity as compared with CD8⁺ T cells that were isolated from mice immunized with OVA alone or OVA plus LPS (FIGS. 2B, 8E).

To compare the antigen-specificity of T cells that result from an immunization with activation stimuli, pyroptotic stimuli or hyperactive stimuli, mice were injected with OVA, alone or with activating stimuli (LPS), with pyroptotic stimuli (LPS+alum) or with hyperactivating stimuli (LPS+oxPAPC or PGPC). Alternatively, mice were immunized with LPS+PGPC without the OVA antigen. 7 days post immunization, CD4⁺ and CD8⁺ T cells were isolated from the skin dLN of immunized mice and were re-stimulated ex vivo for 7 days with naïve BMDC loaded (or not) with OVA to enrich the OVA-specific T cell subset. T cell effector function of OVA-specific T cells was assessed by intracellular staining for IFNγ. TCR specificity was assessed by staining with MHC-restricted OVA peptide tetramers. H2^{kb} restricted SIINFEKL (OVA 257-264) peptide and I-A(d) OVA peptide (OVA 329-337) tetramers were used. The frequency of tetramer⁺IFNγ⁺ double positive cells was measured for CD4⁺ and CD8⁺ T cell subsets. As expected, T cells isolated from mice immunized with LPS+OVA led to enrichment OVA-specific T cells in vitro, and induced higher IFNγ effector function for CD8⁺ T cells as compared to mice immunized with OVA alone (FIG. 2C upper panel). Similarly, CD4⁺ T cells isolated from mice immunized with LPS+OVA displayed higher OVA-specific IFN+ T cells in the presence of high OVA antigen load, as compared to T cells from mice immunized with OVA alone (FIG. 2C lower panel).

Strikingly, OVA with hyperactive stimuli (LPS+OxPAPC or PGPC) were superior at inducing antigen-specific T cells, and resulted in the generation of a higher frequency of tetramer⁺IFNγ⁺ responses upon CD4⁺ or CD8⁺ T cells re-stimulation with OVA antigen (FIG. 2C). Pyroptotic stimuli (LPS+alum) were the weakest inducers of antigen-specific IFNγ responses (FIG. 2C). These results are in accordance with results described herein and elsewhere (M. J. Newman, et al. J. Immunol., vol. 148, no. 8, pp. 2357-62, April 1992; J. M. Brewer, et al. J. Immunol., vol. 163, no. 12, pp. 6448-54, December 1999; A. Mori, et al. Eur. J. Immunol., vol. 42, no. 10, pp. 2709-2719, October 2012), which indicated that alum is an adjuvant that is effective for promoting humoral immunity and TH2 responses, but not TH1 or CTL responses.

Previous studies showed that recombinant IL-1β enhances antigen-specific T cell responses during immunizations, and increases the competence of weak vaccines (S. Z. Ben-Sasson, K. et al. Cold Spring Harb. Symp. Quant. Biol., vol. 78, no. 0, pp. 117-124, January 2013; S. Z. Ben-Sasson, et al. J. Exp. Med., vol. 210, no. 3, pp. 491-502, March 2013). To determine if the antigen-specific T cell responses generated with hyperactive stimuli are dependent on the inflammasome-IL-1β axis, side-by-side comparisons of T cell activity following immunization with hyperactive stimuli in WT versus NLRP3^{-/-} mice was performed. Hyperactive conditions induced reduced tetramer⁺IFNγ⁺ responses in NLRP3^{-/-} as compared to WT mice (FIG. 2C). These results reveal that in hyperactive conditions, NLRP3 activation is critical for antigen-specific T cells generation and effector function.

In a recent study of bacterial infections, the white adipose tissue was shown to constitute a reservoir compartment for antigen-specific memory T cells which persist for several months after antigen contraction (S.-J. Han, et al. Immunity, vol. 47, no. 6, p. 1154-1168.e6, 2017). To test the ability of the different DC activation stimuli to induce memory T cells in the subcutaneous adipose tissue, mice were immunized subcutaneously as previously described with OVA and the distinct activation stimuli. 8 days post-immunization, the presence of CD44⁺ memory T cells in the skin inguinal adipose tissue was assessed by flow cytometry. Interestingly, hyperactive conditions induced a higher frequency of CD44⁺ memory T cells, as compared to activation stimuli (FIG. 2D). Of note, no T cells were detected in the adipose compartment when mice were immunized with OVA and pyroptotic stimuli (FIG. 2D). In addition, memory T cells accumulation depended on the activation of NLRP3 inflammasome, as NLRP3^{-/-} mice displayed reduced frequency of CD44⁺ memory T cells (FIG. 2D). Overall, these results indicate that memory T cells generated by hyperactive stimuli are not only confined to the skin dLN of immunized mice, but also induce memory T cells in the subcutaneous adipose tissue compartment.

Altogether, these data provide evidence that hyperactivating stimuli generate a large population of functional memory CD4⁺ and CD8⁺ T cells, with a strong bias towards TH1 responses.

Hyperactive DCs can Use Complex Antigen Sources to Stimulate T Cell Mediated Anti-Tumor Immunity.

Based on the findings described above, it was reasoned that hyperactive stimuli may be particularly useful in strategies where immunizations have been difficult to achieve clinical (or pre-clinical) benefits. One area of interest relates to cancer immunotherapy. Current efforts to stimulate anti-tumor immunity include strategies that invigorate resident T cell populations (e.g. PD-1 blockade) or personalized cancer vaccine strategies that stimulate the generation of de novo T cell responses to tumor-specific antigens (TSAs) (REFs). This latter effort has been hampered by the inability to use tumor cell lysates as a source of TSAs, also known as neo-antigens. Consequently, efforts are underway to improve the identification of neo-antigens, which can be used in pure form to elicit T cell mediated anti-tumor immunity. While these efforts have yielded successes (D. B. Keskin, et al., Nature, vol. 565, no. 7738, pp. 234-239, January 2019; Z. Hu, P. A. Ott, and C. J. Wu, Nat. Rev. Immunol., vol. 18, no. 3, pp. 168-182, December 2017; P. A. Ott, et al. Nature, vol. 547, no. 7662, pp. 217-221, 2017), the path to neo-antigen identification requires a pipeline for mutated, as well as aberrantly expressed TSAs discovery (C. M. Laumont, et al. Sci. Transl. Med., vol. 10, no. 470, p. eaau5516, December 2018), which is laborious and not representative of the natural course of events. Naturally, DCs never encounter pure antigens, but can rather stimulate T cell responses in complex environments.

It was reasoned that since hyperactive DCs are superior stimulators of T cell mediated responses, then perhaps hyperactivating stimuli would allow us to bypass the need for neo-antigen identification and permit the use of whole tumor cell lysates (WTL) as an antigen source. WTL serves as an attractive alternative source of antigens, as these lysates provide a spectrum of mutated and aberrantly expressed TSAs and may enable the generation of a broad repertoire of T cells specific to tumor associated antigens.

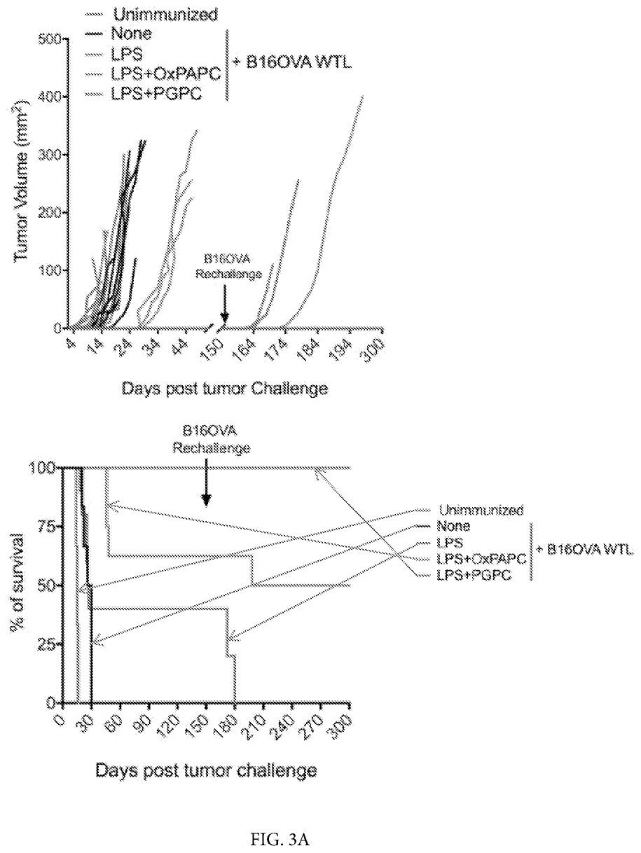

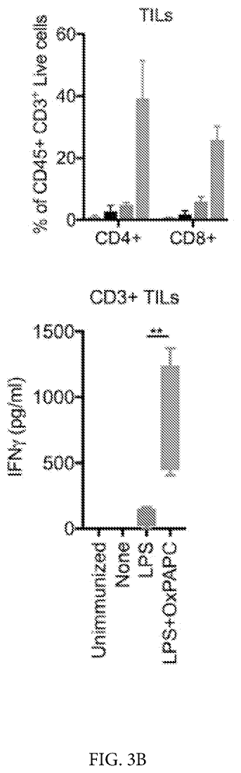

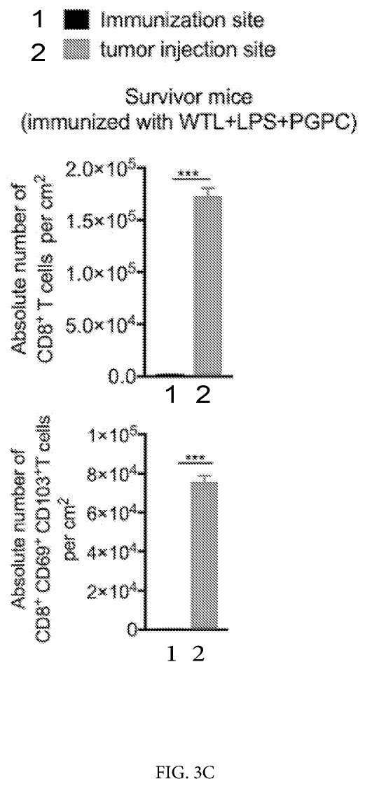

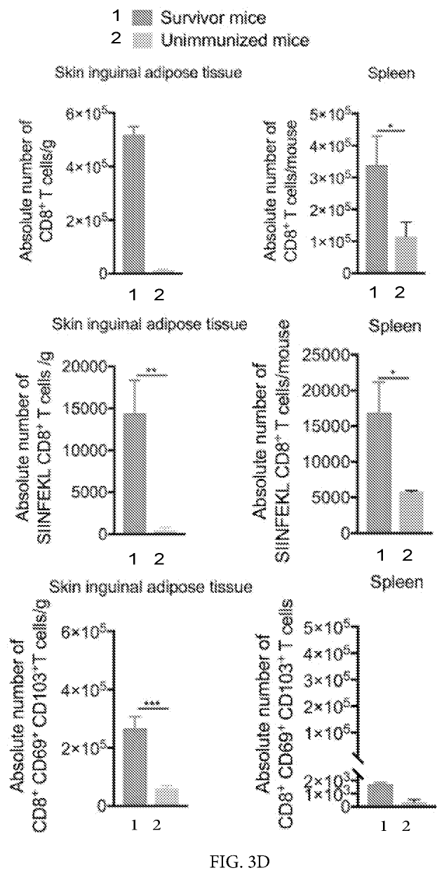

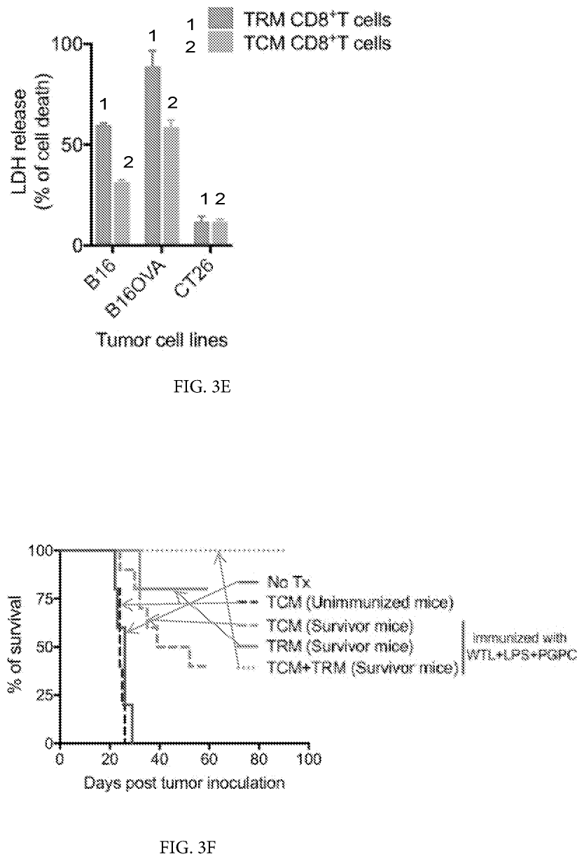

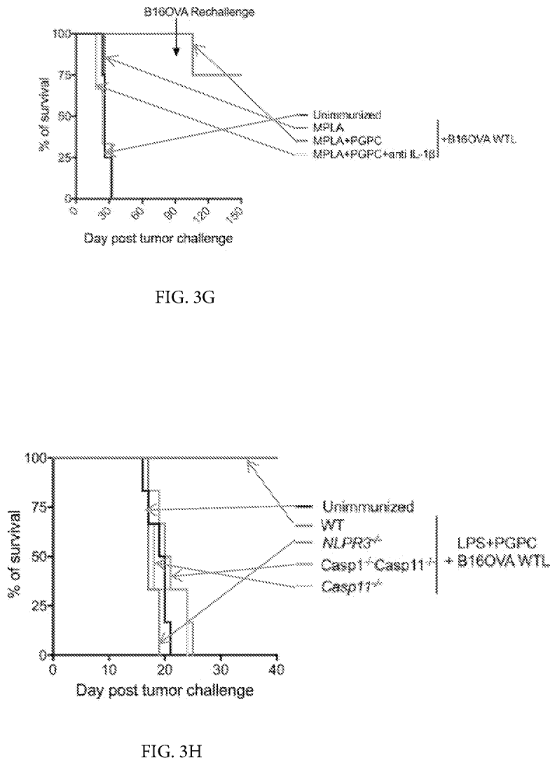

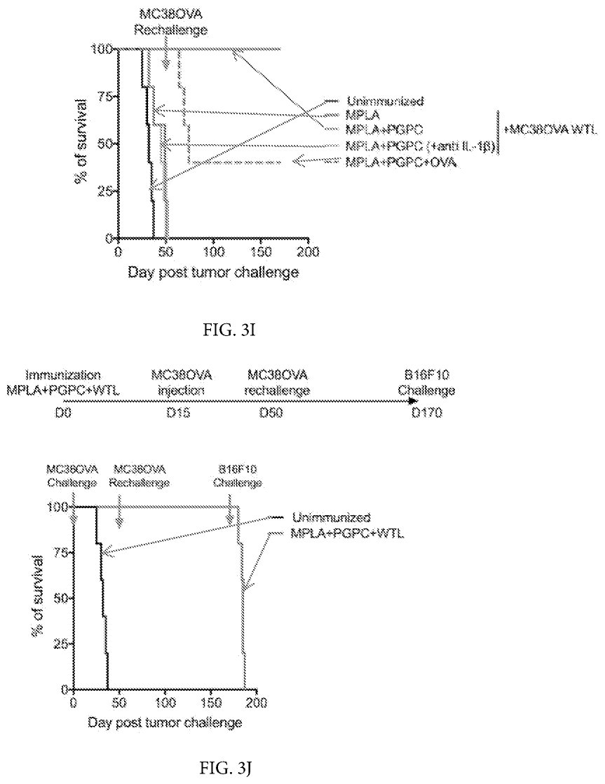

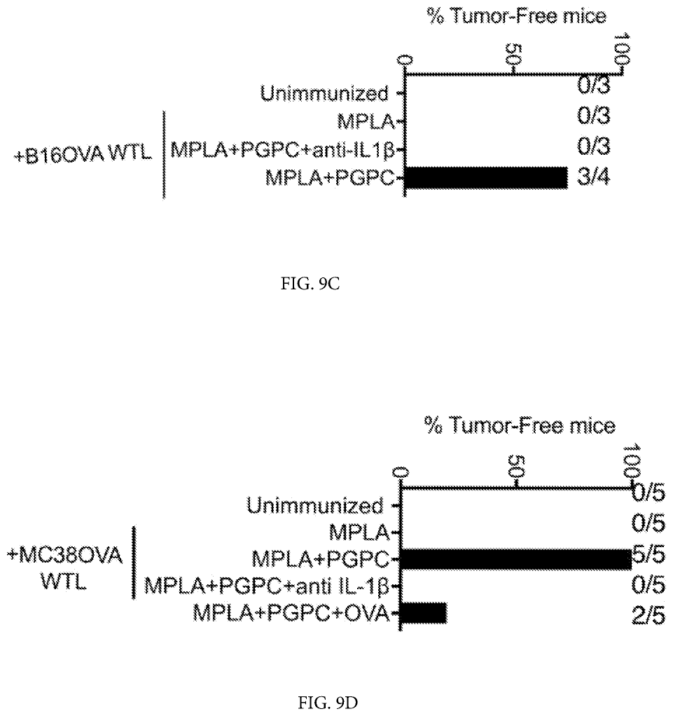

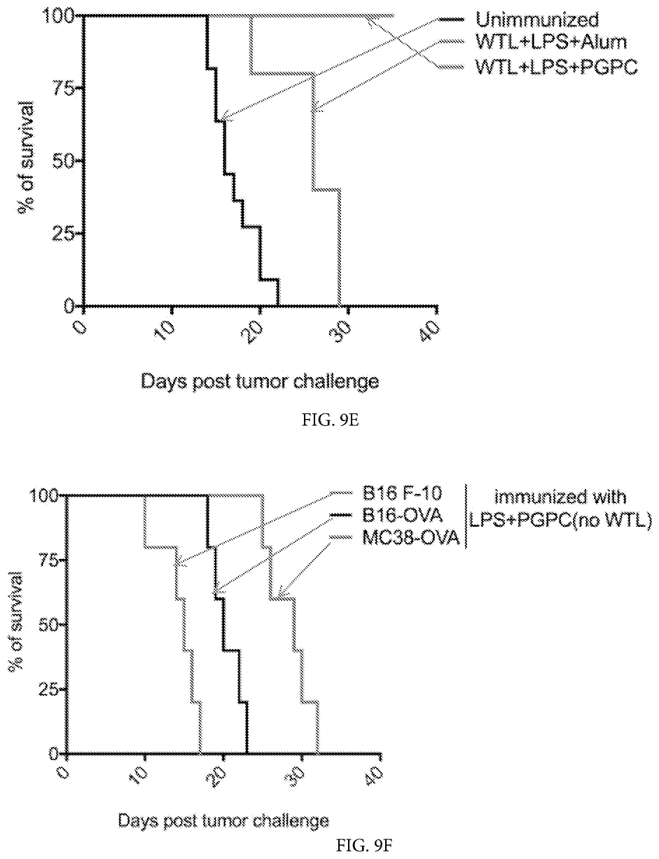

To address the possibility that hyperactivating stimuli can adjuvant WTL, mice were immunized on the right flank with WTL alone, or WTL mixed with the activating stimulus LPS or the hyperactivating stimuli LPS+oxPAPC or LPS+PGPC. The source of the WTL was B16 melanoma cells expressing OVA (B16OVA). Fifteen days post-immunization, mice were challenged subcutaneously (s.c) on the left upper back with the parental B16OVA cells. Unimmunized mice or mice immunized with WTL alone did not exhibit any protection, and all mice harbored large tumors by day 24 after tumor inoculation and died (FIG. 3A). Similarly, WTL+LPS immunizations offered minimal protection. Two of eight mice immunized with WTL+LPS were tumor-free, but quickly relapsed after B160VA re-challenge (FIG. 3A), indicating no protective immunity was conferred by stimuli that merely activate DCs. In contrast, WTL immunizations in the presence of LPS and oxPAPC induced a significant delay in the tumor growth and resulted in a strong protection against subsequent lethal re-challenge with parental B160VA tumor cells; 50% of the immunized mice were fully protected (FIGS. 3A and 9A).

To determine if the protective responses induced by hyperactive stimuli correlated with T cell responses, tumors were harvested from mice receiving each activation stimulus. Tumors from mice immunized with hyperactivating stimuli contained a substantial abundance of CD4⁺ and CD8⁺ T cells (FIG. 3B). Upon anti-CD3 and anti-CD28 stimulation, enriched T cells from these tumors secreted high amounts of IFNγ (FIG. 3B). Thus, the superior restriction of tumor growth induced by hyperactivating stimuli (LPS+oxPAPC) was coincident with inflammatory T cell infiltration into the tumor.

Notably, the protective phenotypes of oxPAPC were superseded by those elicited by the pure oxPAPC component PGPC. WTL immunizations in the presence of LPS+PGPC led to 100% of mice being tumor-free for 150 days post-tumor challenge. These mice completely rejected a lethal re-challenge with B16OVA cells and remained tumor-free 300 days post initial tumor challenge (FIGS. 3A and 9A).

Among memory T cell subsets, T resident memory cells TRM are defined by the expression of CD103 integrin along with C-type lectin CD69, which contribute to their residency characteristic in the peripheral tissues (REFs). CD8⁺ TRM cells have recently gained much attention, as these cells accumulate at the tumor site in various human cancer tissues and correlated with the more favorable clinical outcome (J. R. Webb, et al. Clin. Cancer Res., vol. 20, no. 2, pp. 434-444, January 2014; F. Djenidi, et al. J. Immunol., vol. 194, no. 7, pp. 3475-86, April 2015; S. L. Park, et al. Nature, vol. 565, no. 7739, pp. 366-371, January 2019). In an experimental cutaneous melanoma model, CD8⁺ TRM cells in the skin promoted durable protection against melanoma progression.

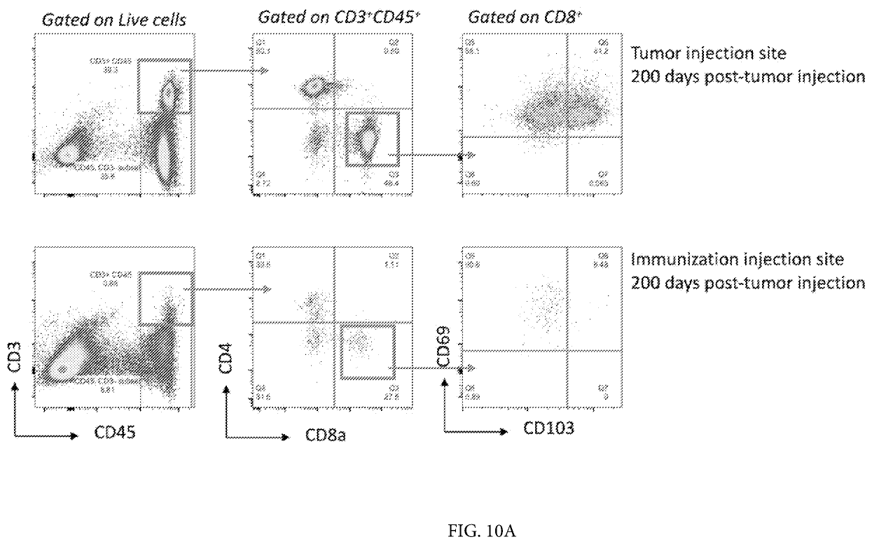

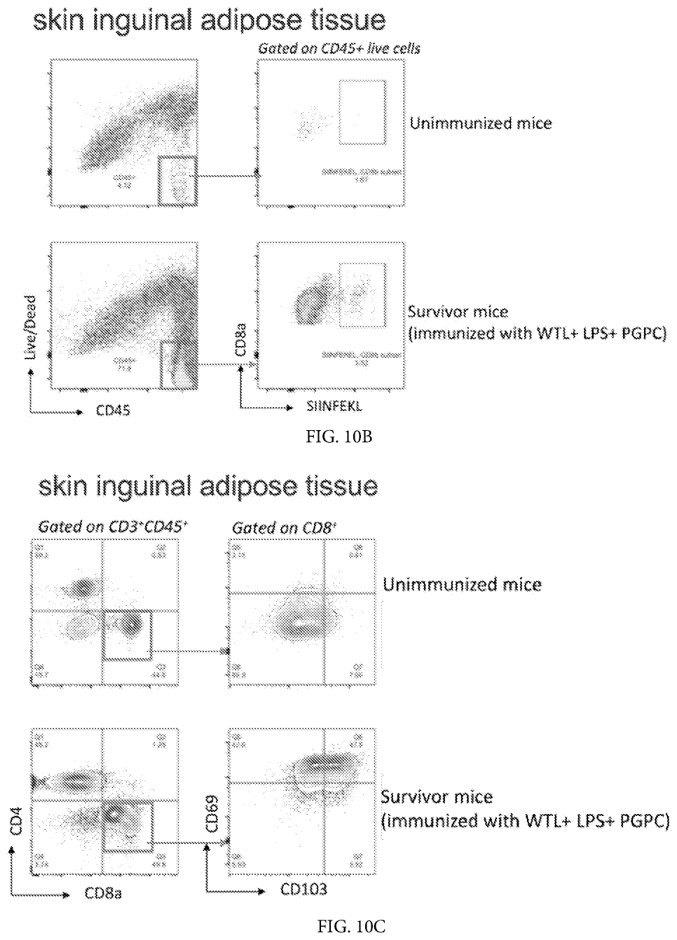

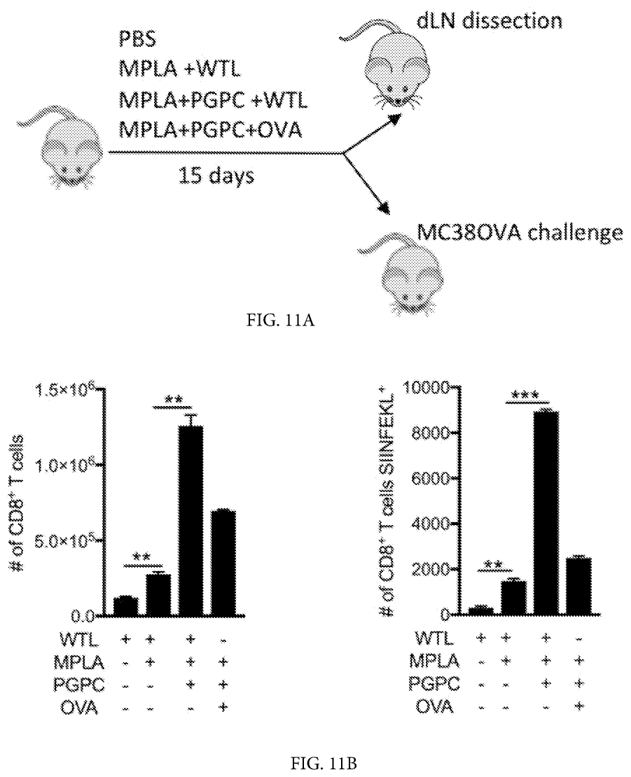

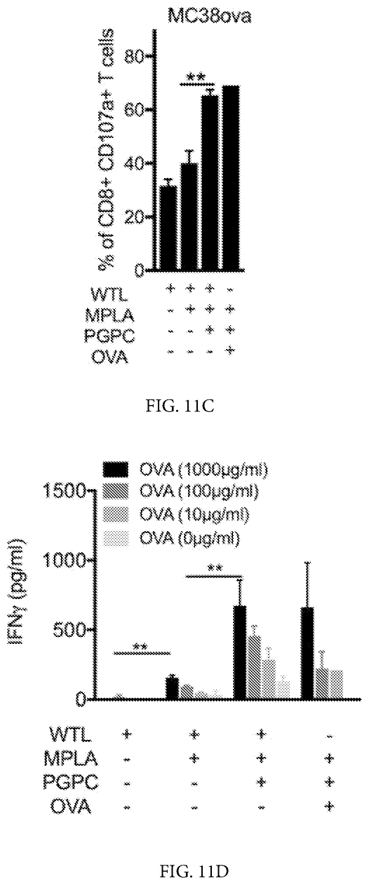

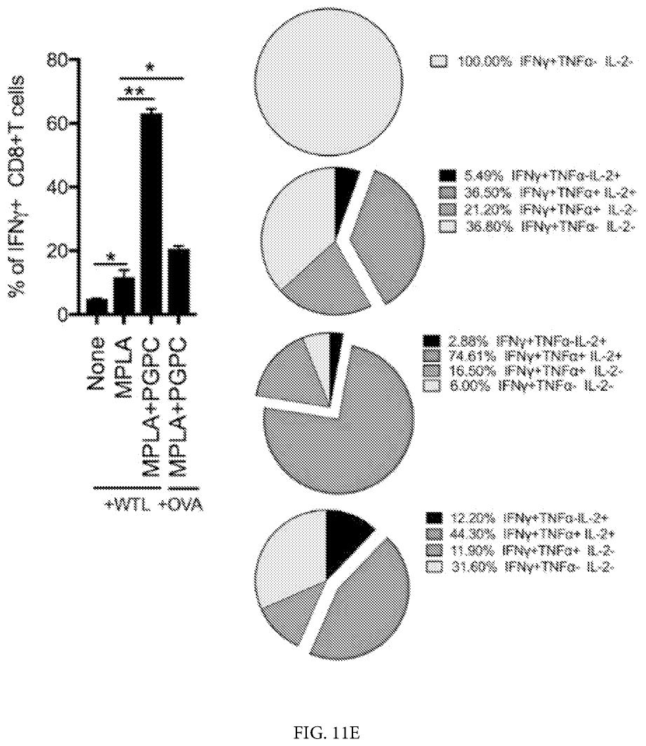

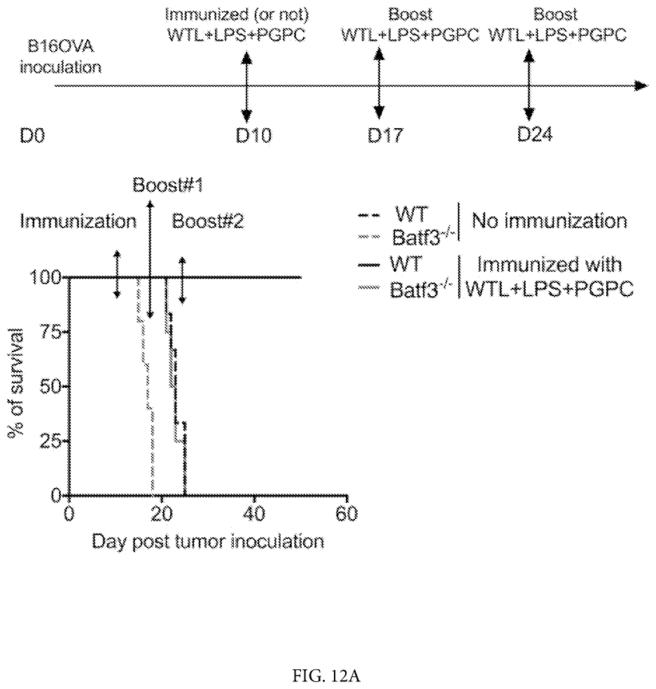

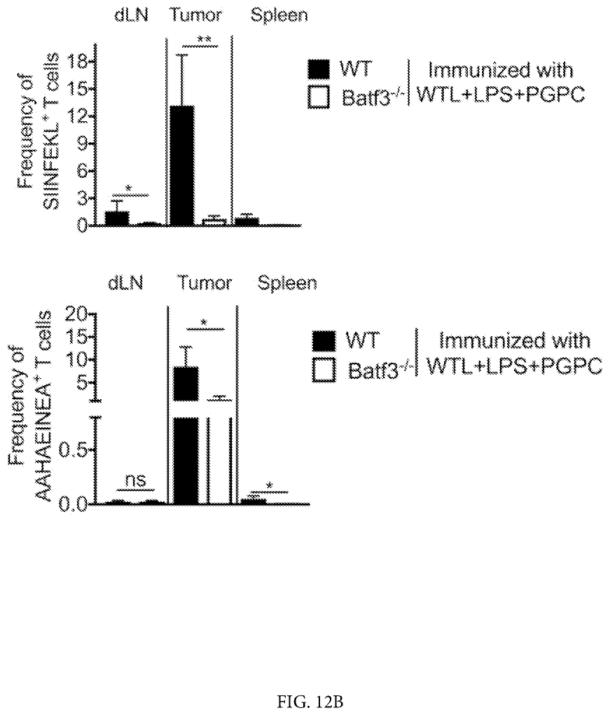

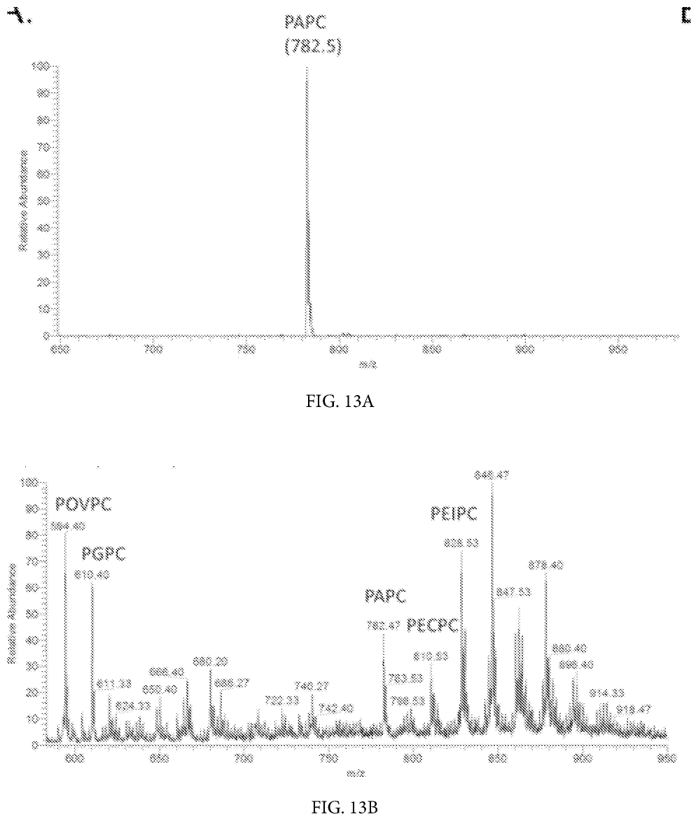

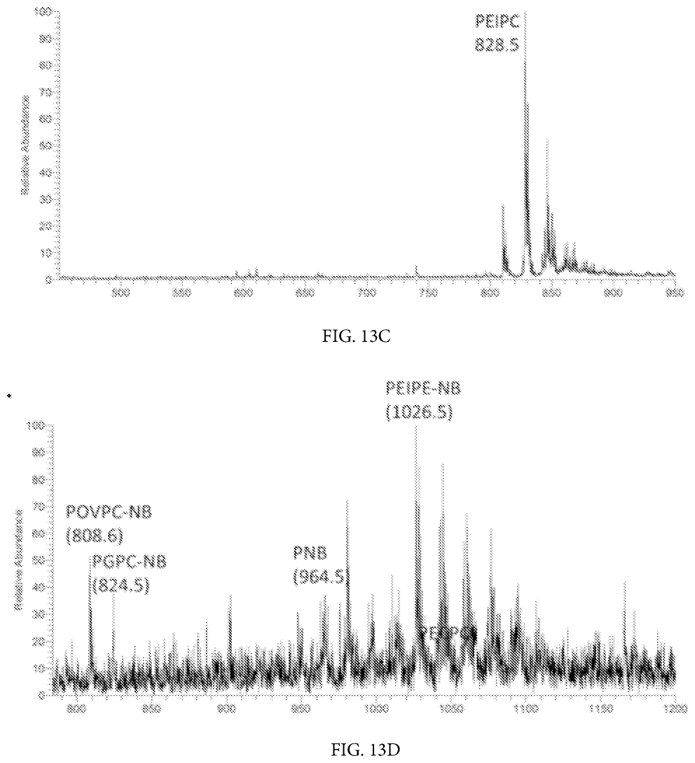

The presence of TRM cells at the tumor injection site, as well as the immunization skin biopsies in the survivor mice that were previously immunized with the hyperactivating stimuli LPS+PGPC, was examined. Interestingly, 200 days post-tumor inoculation, CD8⁺ CD69⁺ CD103⁺ TRM cells were highly enriched at the site of tumor injection, but were scarce at the immunization site in all survivor mice (FIGS. 3C and 10A). These data are in accordance with clinical and experimental reports that correlate the presence of high levels of TRM with a long-term tumor control, in which TRM are potentially maintained for long periods to survey tumor injection site (S. L. Park, et al. Nature, vol. 565, no. 7739, pp. 366-371, January 2019; C. M. Koebel, et al. Nature, vol. 450, no. 7171, pp. 903-907, December 2007).

CD8⁺ TRM cells accumulate in the white adipose tissues after antigen contraction, and are mobilized to sites of infections upon secondary challenge (S.-J. Han et al., Immunity, vol. 47, no. 6, p. 1154-1168.e6, 2017). The T cell compartment in the subcutaneous adipose tissue surrounding the inguinal LN that drains the site of immunization, was analyzed. A high frequency of CD8⁺ T cells were observed, as well as antigen-specific CD8⁺ TRM cells in the adipose tissue of mice immunized with WTL and the hyperactivating stimuli LPS+PGPC (FIGS. 3D left panels and 10B, 10C). In contrast, few TRM cells were observed in the inguinal adipose tissue of unimmunized mice (FIGS. 3D left panels and 10B, 10C). Additionally, circulating memory T cells from the spleen of survivor mice immunized with WTL and the hyperactivating stimuli LPS+PGPC, contained a large population of OVA-specific T cells, as compared to their unimmunized counterparts.

To examine the functional specificity of these T cells, we monitored cytotoxic lymphocyte (CTL) activity ex vivo. Circulating memory CD8⁺ T cells and TRM cells were isolated from the spleen or the skin adipose tissue of survivor mice that previously received hyperactivating stimuli. These cells were cultured with B16OVA cells, or B16 cells not expressing OVA or an unrelated cancer cell line CT26. CTL activity, as assessed by LDH release, was only observed when CD8⁺ T cells were mixed with B16OVA or B16 cells (FIG. 3E). No killing of CT26 cells was observed (FIG. 3E), thus indicating the functional and antigen-specific nature of hyperactivation-induced T cell responses.

Based on the antigen-specific T cell responses induced by hyperactivating stimuli, it was determined whether T cells were sufficient to protect against tumor progression. CD8⁺ T cells were transferred from survivor mice into naïve mice and subsequently challenged with the parental tumor cell line used as the initial immunogen. Transfer of CD8⁺ TRM or circulating CD8⁺ T cells from survivor mice into naïve recipients conferred profound protection from a subsequent tumor challenge, with the TRM subset playing a dominant protective role (FIG. 3F). Transfer of both T cell subsets from survivor mice into naïve mice, one week before tumor inoculation, provided 100% protection of recipient mice from subsequent tumor challenges (FIG. 3F). These collective data indicate that PGPC is a major bioactive hyperactivation stimulus, which confers optimal protection in a B16 melanoma model by inducing strong circulating as well as resident anti-tumor CD8⁺ T cell responses.

To determine if the benefits of PGPC-based hyperactivating stimuli extend to other murine models of cancer, similar experiments were performed with the parental B16-F10 melanoma cells. Unlike B16OVA melanomas, which contain an abundant neo-antigen in the form of OVA, with few exceptions (J. C. Castle et al., Cancer Res., vol. 72, no. 5, pp. 1081-91, March 2012; M. O. Mohsen et al., Front. Immunol., vol. 10, p. 1015, 2019), the tumor-specific antigens present in B16-F10 cells are less-defined. B16-F10 cells therefore represent a common clinical scenario where minimal neo-antigen identification has occurred. Notably, PGPC-based strategies of hyperactivation induced protection against B16-F10 tumor growth (FIG. 9B).

Similar findings were made when we replaced LPS+PGPC with MPLA+PGPC; 100% of mice immunized in this manner remained tumor-free for 90 days post-tumor challenge, and 75% of the mice rejected a lethal re-challenge with B160VA cells (FIG. 3G). These data indicate that unlike activating stimuli, hyperactive stimuli are capable of adjuvanting complex antigen mixtures (e.g. WTL) to promote long-lasting and protective anti-tumor immunity.

Hyperactivating stimuli induce inflammasome-dependent anti-tumor immunity. A defining attribute of hyperactivating stimuli is their ability to induce IL-1β secretion from living cells. To determine if IL-1β and its upstream inflammasome regulators are important for anti-tumor responses, several manipulations were performed. First, immunizations were performed as described above, expect that intravenous (i.v.) injections of neutralizing anti-IL-10 antibodies were performed two days prior to immunization, followed by three consecutive injections on day 0, day 1 and day 2 post-immunization to ensure chronic depletion of IL-1β. 15 days after immunization, mice were challenged with the parental tumor cells. Neutralizing IL-1β completely abolished the protection of mice against tumor growth in the B16OVA melanoma model (FIGS. 3G and 9C). Similar to the phenotype observed upon IL-1β neutralization, hyperactive stimuli conferred no protection from tumor challenge when immunizations were performed in NLRP3^{-/-}, Casp1^{-/-}/11^{-/-} or Casp11^{-/-} mice (FIG. 3H). Similar results were obtained using a distinct cancer model—the MC38 colon adenocarcinoma model (FIGS. 31 and 10D), where IL-1β neutralization eliminated the ability of hyperactivating stimuli to protect against challenges with an MC38 line expressing OVA (MC380VA). These functional data establish a link between the activities of hyperactivating stimuli in vitro and the T cell promoting activities of these stimuli in vivo. Notably, not all NLRP3 agonists conferred anti-tumor immunity, as LPS+alum was unable to adjuvant WTL to protect against tumor growth (FIG. 10E). These findings underscore the importance of hyperactive DCs (not pyroptotic DCs) in the induction of anti-tumor immunity.

Hyperactive stimuli can adjuvant WTL or neo-antigens to induce anti-tumor immunity. The ability of hyperactivating stimuli to induce protective immunity to complex antigen mixtures raises the question of how well these protective responses perform, as compared to immunizations with pure neo-antigens. To address this question, side-by-side immunizations were performed with pure OVA or OVA present in a tumor lysate. Mice were injected with WTL from MC380VA cells in the presence of absence of the activating stimulus MPLA or the hyperactivating stimulus MPLA+PGPC. These injections were compared to those where WTL was replaced with pure OVA as an antigen. Fifteen days post-immunization, mice from each group were blindly separated into two sister cohorts. One cohort received a challenge with MC380VA cells while the other was sacrificed and dissected to assess the CD8⁺ T cell responses (FIG. 11A). It was found that hyperactive stimuli induced higher absolute number of CD8⁺ T cells infiltrating the dLN (FIG. 11B left panel) and a higher number of SIINFEKL-specific CD8⁺ T cells, as compared to mice immunized with WTL either alone or with MPLA alone (FIG. 11B right panel). CD8⁺ T cells isolated from the dLN of mice immunized with hyperactive stimuli exhibited the highest degranulation ability upon co-culture with MC380VA cells (FIG. 11C). These cells also produced higher levels of IFNγ upon ex vivo re-stimulation with BMDC loaded with OVA (FIG. 11D). In addition, single cell analysis using intracellular cytokine staining of CD8⁺ T cells stimulated with PMA and ionomycin, revealed highly polyfunctional T cells within the CD8⁺ T cell compartment of mice immunized with hyperactive stimuli. These CD8⁺ T cells were able to simultaneously produce multiple cytokines including IL-2, TNFα and IFNγ (FIG. 11E).

The enhanced T cell activity present in mice immunized with hyperactivating stimuli correlated with survival for over 150 days after challenge with MC380VA cells (FIG. 3I). Of note, as compared with immunizations where pure OVA was used, WTL-based immunizations conferred superior protection after tumor challenge (FIG. 3I). The inability of single antigens to strongly protect from cancer is consistent with recent work demonstrating the value of using multiple neo-antigens (up to 20 peptides) in personal cancer vaccines (P. A. Ott et al., Nature, vol. 547, no. 7662, pp. 217-221, 2017; J. C. Castle et al., Cancer Res., vol. 72, no. 5, pp. 1081-91, March 2012).

To determine if the anti-tumor response generated during hyperactivation-based immunizations in MC38 cancer model was antigen specific, survivor mice were re-challenged with an unrelated tumor cell line. Survivor mice that had rejected MC380VA tumors died quickly following a challenge with B16-F10 cells (FIG. 3J). Furthermore, in all instances of hyperactivation-based anti-tumor immunity, protection against B16-F10, B160VA or MC380VA cells was only conferred if mice received an immunization with the corresponding WTL (FIG. 9F). These findings underscore the ability of hyperactivating stimuli to drive antigen-specific anti-tumor responses, even when the experimentalist is ignorant (and agnostic) to neo-antigen identity.

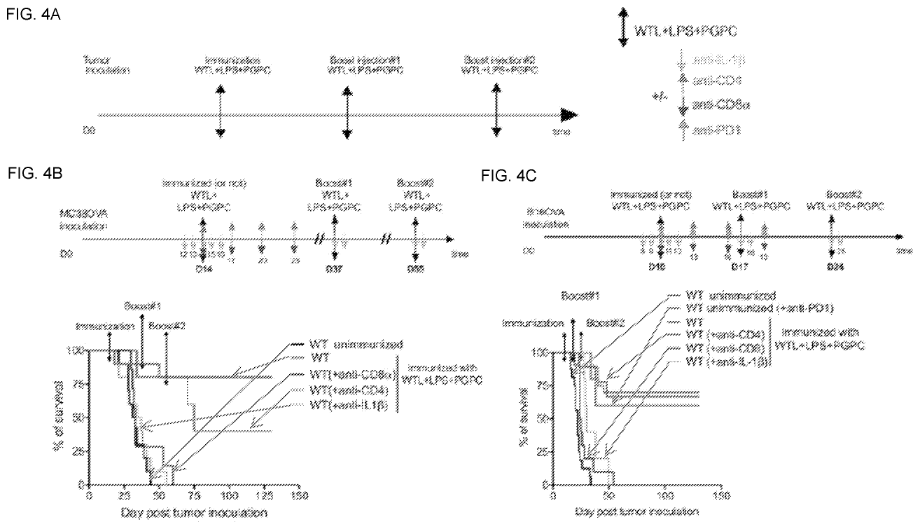

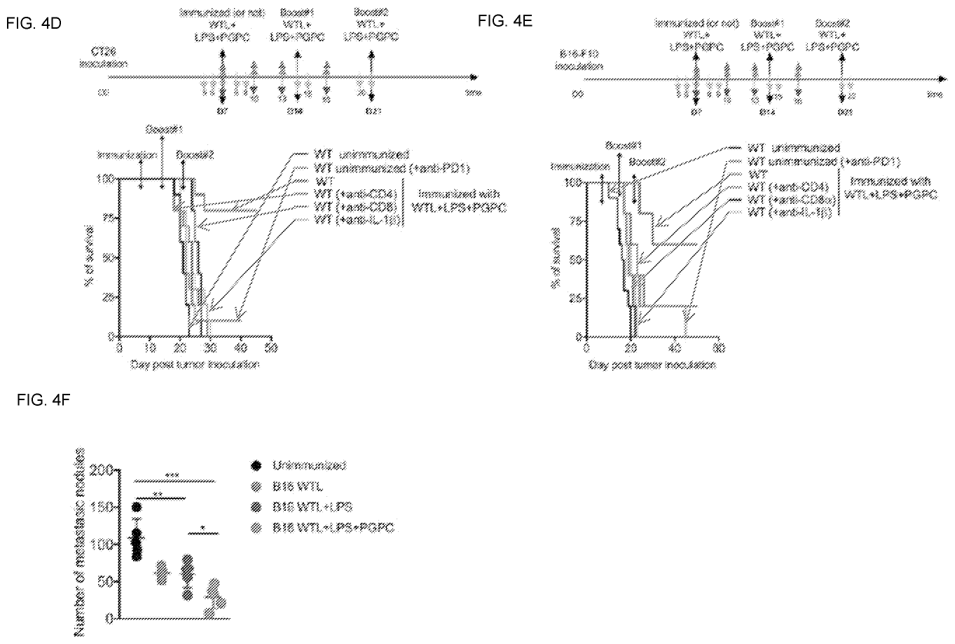

Hyperactive stimuli use WTL to protect against metastasis to the lung. To determine if hyperactivating stimuli could be harnessed as a cancer immunotherapy, anti-tumor responses in mice that harbored a growing tumor prior to any additional treatment, were examined. For these studies, rather than using cultured tumor cells as an antigen source, ex vivo WTL were generated using syngeneic tumors from unimmunized mice, in which 10 mm harvested tumors were dissociated and then depleted of CD45⁺ cells. Mice were inoculated subcutaneously (s.c.) with tumor cells on the left upper back. When tumors reached a size of 3-4 mm, tumor-bearing mice were either left untreated (unimmunized) or received a therapeutic injection on the right flank, which consisted of ex vivo WTL and LPS plus PGPC. Two subsequent s.c. boosts of therapeutic injections were performed (FIG. 4A). Interestingly, these hyperactivation-based therapeutic injections induced tumor eradication in B160VA and B16F10 melanoma models, and in MC380VA and CT26 colon cancer tumor models (FIGS. 4B-4E). In all of these models, a high percentage of mice that received the immunotherapy regimen remained tumor-free long after the tumor inoculation (FIGS. 4B-4E). The efficacy of the immunotherapy was dependent on IL-1β in all the tested tumor models, since the neutralization of IL-1β abolished protection conferred by hyperactivating stimuli plus ex vivo WTL (FIGS. 4B-4E). In addition, CD8⁺ T cells were crucial for protection against immunogenic tumor models such as B16OVA or MC380VA tumors, whereas CD4⁺ and CD8⁺ T cells were both required for protection against less immunogenic tumors such as CT26 and B16F-10 tumors (FIGS. 4B-4E).

To determine how hyperactivation-based immunotherapy compares in efficacy to therapies based on PD-1 blockade, side-by-side assessments were performed.

Hyperactivation-based immunotherapy was as efficient as anti-PD-1 therapy in the immunogenic B16OVA model, but more efficient in tumor models that are insensitive to anti-PD-1 treatment such as CT26 and B16F-10 tumors (FIGS. 4C-4E).

Hyperactivation-based immunotherapy did not only protect mice against implanted tumors subcutaneously. Indeed, hyperactivation-based immunotherapy protected mice against B16 lung metastasis, as compared to immunizations with WTL injections alone or WTL+LPS (FIG. 4F). These observations indicate that hyperactivation-induced an anti-tumor immunity that is not restricted to local responses, but also contribute to a protective systemic immunity.

Hyperactivation of inflammasome-competent DCs is sufficient to confer protective anti-tumor immunity. As DCs are the principal cells responsible for stimulating de novo T cell mediated immunity, it was sought to determine if conditions that specifically hyperactivate DCs is sufficient to confer anti-tumor immunity. This possibility was addressed by performing an adoptive transfer into mice of BMDCs that were stimulated ex vivo with different activation stimuli and WTL. BMDCs were chosen because these cells are 1) well-characterized to become hyperactivated and 2) are considered models for monocyte-derived DCs, which are the most common APCs used in DC-based immunotherapies in humans (R. L. Sabado et al., Cell Res., vol. 27, no. 1, pp. 74-95, January 2017).

BMDCs were treated with various activation stimuli, along with WTL, and were then injected s.c. every 7 days for 3 consecutive weeks into B16OVA tumor-bearing mice.

BMDCs that were activated with LPS and pulsed with B16OVA WTL provided a slight protection from B160VA-induced lethality, as compared to mice injected with naïve BMDCs; 25-30% of mice that received DC transfer rejected tumors and remained tumor-free long after the last/third DC transfer procedure. Notably, hyperactive BMDCs induced a complete rejection of B160VA tumors in 100% of tumor-bearing mice. The anti-tumor activity of hyperactive DCs was dependent on inflammasomes in these cells, as NLRP3^{a} and Casp1^{-/-}11^{-/-} BMDC transfers induced only a minor rejection that was comparable to active DCs. These data therefore indicate that hyperactive DCs are sufficient to induce durable protective anti-tumor immunity, and that inflammasomes within DCs are essential for this process.

### DISCUSSION

In this study, the immunological activities that are upregulated upon treatment of DCs with hyperactivating stimuli were expanded. Not only are these stimuli capable of eliciting IL-1β release from living cells, but hyperactivating stimuli also exceed other activation stimuli in their ability to induce CD40 expression and IL-12p70 secretion. Furthermore, cells exposed to hyperactivating stimuli exhibit enhanced surface expression of MHC-peptide complexes. These collective findings underscore the hyperactive nature of the DCs that are exposed to oxPAPC or its pure component PGPC and provides evidence of an enhanced ability to stimulate adaptive immunity. While it was found that hyperactive DCs are indeed better stimulators of T cell responses than activated or pyroptotic cells, the most notable aspect of their activities may be their ability to stimulate a TH1- and CTL-focused response. Indeed, stimuli that hyperactivate DCs led to a 100:1 ratio of TH1:TH2 cells; no other strategy of DC activation induced such a biased T cell response.

It is noteworthy that alum, a well-defined inflammasome stimulus, does not exhibit the same activities as oxPAPC or PGPC. Indeed, it is well-recognized that alum induces TH2 immunity. These findings were verified in this study, as alum or alum+LPS treatments induced robust TH2 immunity. One possible reason for the lack of TH1-focused immunity of alum-treated cells is based on findings herein that alum is a poor inducer of several signals necessary for TH1 differentiation, such as CD40 expression and IL-12p70 secretion. Notably, even when the DCs that did not undergo pyroptosis in response to alum+LPS were examined, CD40 expression was strikingly low. The lack of high-level expression of these factors likely renders pyroptotic stimuli weak inducers of TH1 responses and consequently, anti-tumor immunity. Without wishing to be bound by theory, it was proposed that the TH1-focused immunity induced by hyperactive DCs result from the actions of inflammasomes, as well as several other features of these cells. These additional features include enhanced antigen presenting capacity, CD40 expression, IL-12p70 expression and increased viability. It is likely that each of these enhanced activities are important for DC functions as APCs and likely contribute to the strong TH1-focused immune responses observed under conditions of DC hyperactivation.

These results may help explain why certain chemotherapeutic agents (e.g. oxaliplatin) induce tumor cell death and inflammasome-dependent anti-tumor T cell immunity (F. Ghiringhelli et al., Nat. Med., vol. 15, no. 10, pp. 1170-1178, 2009). Oxaliplatin is a robust stimulator of reactive oxygen species (ROS) production, which can oxidize biological membranes and create a complex mixture of distinct oxidized phospholipid species including PGPC. It is therefore possible that the protective immunity induced by oxaliplatin results from the actions of hyperactive DCs that prime anti-tumor T cell responses.

It was found that hyperactive stimuli could be harnessed as an immunotherapy using complex mixtures of antigen. WTL is an attractive source of antigens for several reasons, not the least of which is from a practical perspective. A significant benefit of WTL-based approaches is that it alleviates the need for neo-antigen identification. Despite the potential benefits offered by WTL-based immunotherapies, prior work in this area has yielded mixed results. The finding herein, is that hyperactivating stimuli are uniquely capable of adjuvanting WTL to elicit potent anti-tumor immunity may explain the lack of success in prior work, as the strategies of DC activation discovered herein, have not before been considered. On this latter point, it is noteworthy that DC hyperactivating strategies can protect mice from lethality associated with tumors that are sensitive to PD-1 blockade and those that are resistant to PD-1 blockade. The full spectrum of tumors amenable to treatment by hyperactivating stimuli is undefined, but these studies provide a mandate to further explore the value of DC-centric strategies of cancer immunotherapy.

### Example 2: cDC1 Control Tumor Rejection Induced by Hyperactivation-Based Immunotherapy

Based on our previous findings that hyperactive DCs are superior stimulators of antigen-specific Th1 and CTL responses, we reasoned that hyperactivating stimuli could be harnessed as an immunotherapeutic strategy for cancer. To examine the effect of hyperactive-stimuli injection on anti-tumor immunity, tumor-bearing mice (harboring a tumor of 4 mm on the right flank) received a s.c. injection with ex vivo whole tumor lysates (WTL) and the hyperactivating stimuli LPS+PGPC on the left flank. Two subsequent s.c. boost injections with whole tumor lysates (WTL) and LPS+PGPC were performed. Hyperactivation-based injections induced tumor rejection in B160VA, and a high percentage of WT mice that received the immunotherapy regimen remained tumor-free after the tumor inoculation (more than 40 days). The hyperactivation induced protection was dependent on cDC1 cells, as Batf3^{-/-} mice (lacking cDC1 cells) that received the immunization regimen failed to induce tumor control. Furthermore, in contrast to immunized WT mice which displayed a high frequency of tumor-specific CD4⁺ and CD8⁺ T cells infiltrating the tumor-injection site, Batf3^{-/-} mice lacked OVA-specific CD8+ T cells and displayed lower frequency of OVA-specific CD4⁺ T cells in the tumor microenvironment. Overall, these data indicate that: 1) the hyperactivation-stimuli (LPS+PGPC) is uniquely capable of adjuvanting WTL to elicit potent anti-tumor immunity, and that 2) cDC1 cells are the major antigen-presenting cells that initiate hyperactivation-induced anti-tumor immunity in vivo.

### Example 3: Oxidized Phospholipids Induce Hyperactive cDC1 and cDC2 Cells

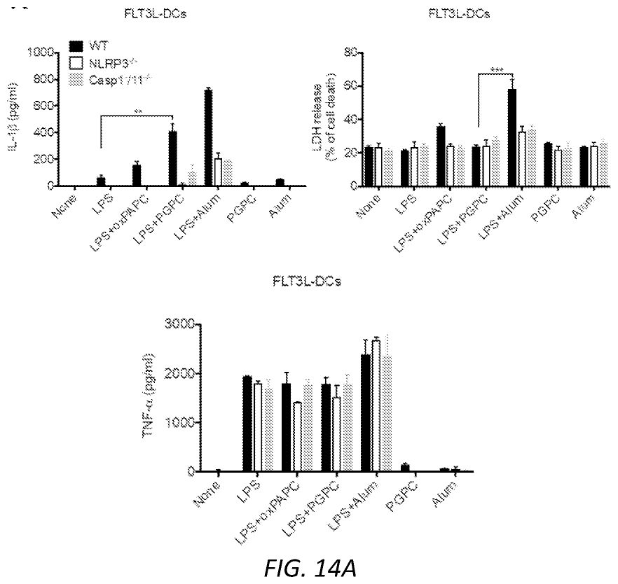

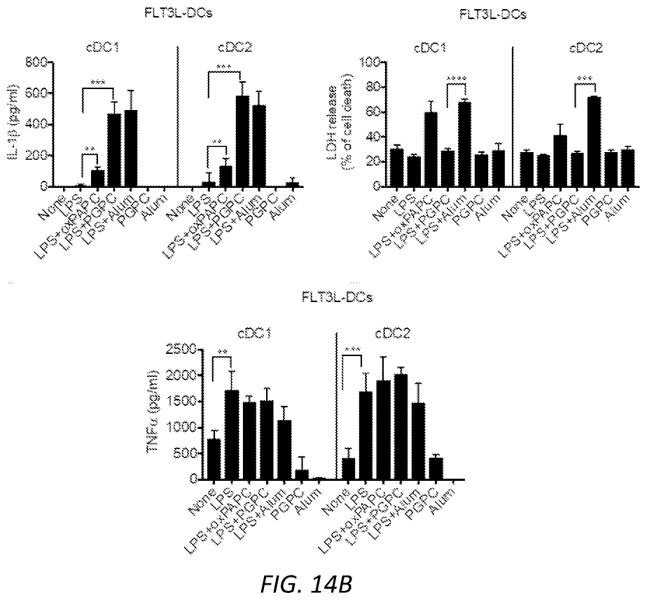

Virtually all studies assessing the state of cell-hyperactivation have focused on the ability of bone marrow derived DC (BMDCs), generated with the cytokine granulocyte-macrophage colony-stimulating factor (GM-CSF), to release IL-1β while retaining viability [24,31,32,33,34]. Recent report demonstrated that monocyte-derived macrophages, rather than DCs, are responsible for inflammasome activation and IL-1β secretion [35]. To examine whether conventional DCs can achieve a hyperactivation state, we used BMDCs generated using the DC hematopoietin Fms-like tyrosine kinase 3 ligand (Flt3L). To assess hyperactivation, FLT3-DCs were primed with LPS and subsequently treated with the oxidized phospholipids oxPAPC or a pure lipid component of oxPAPC named PGPC [36]. Alternatively, FLT3-DCs were stimulated with traditional activation stimuli such as with LPS alone, or FLT3-DCs were primed with LPS then treated with pyroptotic stimuli such as alum. In contrast to traditional activation stimuli, which did not induce IL-1β release from DCs, pyroptotic DCs promoted IL-1β release into the extracellular media (FIG. 14A). IL-1β secretion was co-incident with cell death in pyroptotic DCs, as assessed by the release of the cytosolic enzyme lactate dehydrogenase (LDH) (FIG. 14B). Interestingly, stimulation with the hyperactive stimuli LPS+PGPC, or to a lesser extent with LPS+oxPAPC induced IL-1β secretion from DCs, which occurred in the absence of LDH release (FIG. 14A). All DCs primed or stimulated with LPS promoted the secretion of the cytokine TNFα (FIG. 14A). IL-10 secretion in pyroptotic or hyperactive DCs was in both cases dependent on the inflammasome components NLRP3 and Caspase1/11 (FIG. 14A). These findings are in accordance with prior work, which defined the mechanisms underlying the hyperactive state of DCs, in which oxPAPC bind and stimulate the cytosolic PRR caspase-11, resulting in NLRP3 activation and the assembly of a non-pyroptotic inflammasome that lead to the release of IL-1β from living cells [24]. Similar behaviors of DCs were observed when DCs were primed with other TLR agonists such as TLR9 agonist CpG (FIG. 19A). Thus, these data indicate that FLT3 DCs can achieve a state of hyperactivation. DCs are divided into two major subsets named cDC1s and cDC2s. cDC1 are classical DCs that can cross-present tumor-associated antigens and prime CD8+ T cells [37], [38]. On the other hand, cDC2s govern type 2 immune responses, against parasites in which they activate Th2 immunity. To determine if the behavior of hyperactive DCs extends to cDC1 or cDC2, we isolated cDC1 or cDC2 from FLT3-DCs or from the spleen of wild type naïve mice (FIG. 19B). Similar to the behavior of FLT3-derived DCs, treatment with LPS and PGPC and to a lesser extend with LPS and oxPAPC, led to the release of TNFα and IL-1β from FLT3-derived cDC1s and cDC2 in the absence of cell death, as assessed by LDH release (FIG. 14A). These data indicate that PGPC is the bioactive component of oxPAPC that induces cDC1 and cDC2 hyperactivation. We also observed a similar behavior of splenic cDC2, which produced IL-1β in response to the pyroptotic stimuli LPS and alum concomitant with pyroptotic cell death, but also in response to the hyperactivating stimuli LPS and PGPC in the absence of cell death (FIG. 19C). On the contrary, splenic cDC1 produced minimal amount of IL-1β in response to pyroptotic or hyperactivating stimuli, since these cells were very sensitive to cell death post-sorting, and were unable to get primed by LPS (FIG. 19C). Overall, these results indicate that hyperactivating stimulus can be used to induce IL-1β release from living DCs that have been differentiated in vitro or in vivo. For practical reasons, we continued using in this paper FLT3-derived DCs as a source of DCs.

### Example 4: Hyperactive DCs Potentiate CTL Responses in an Inflammasome Dependent Manner

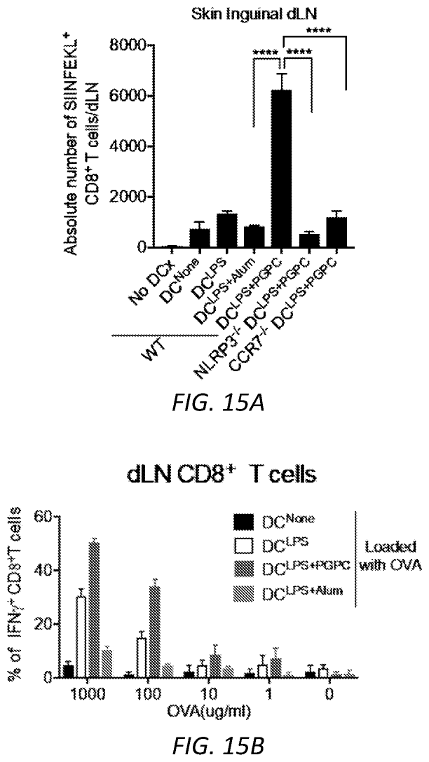

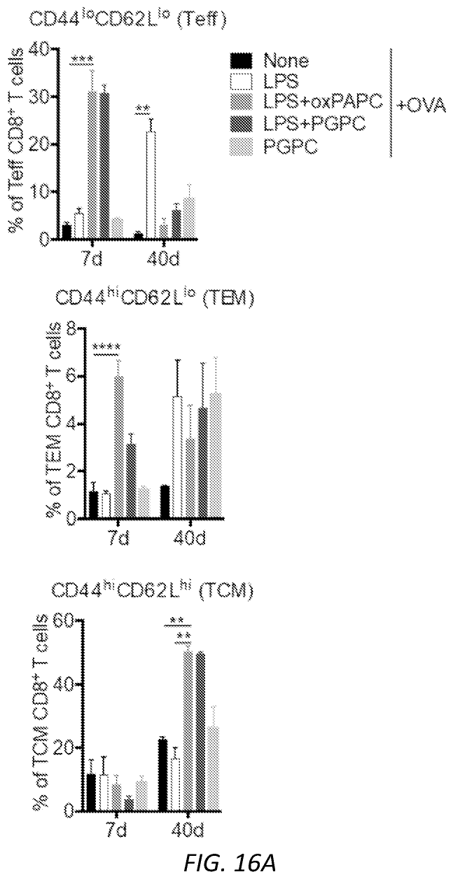

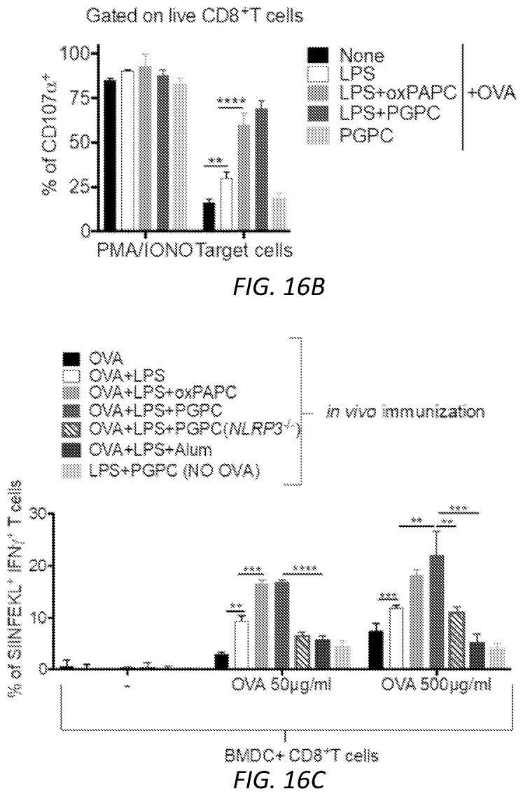

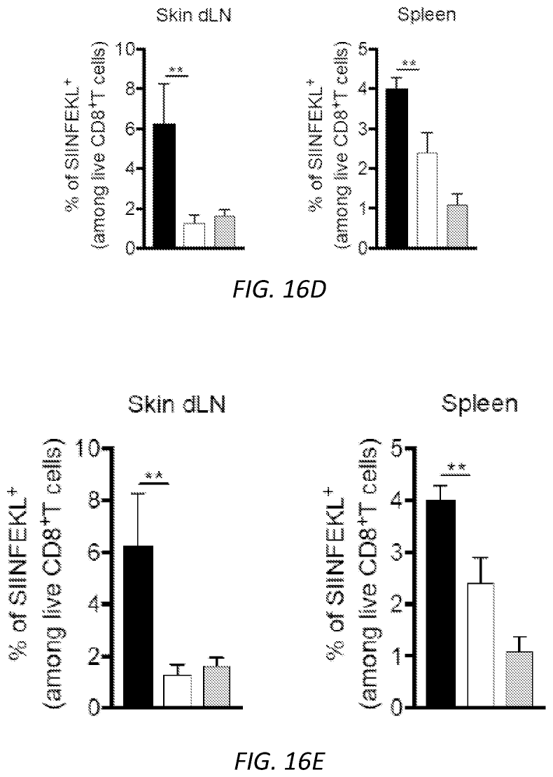

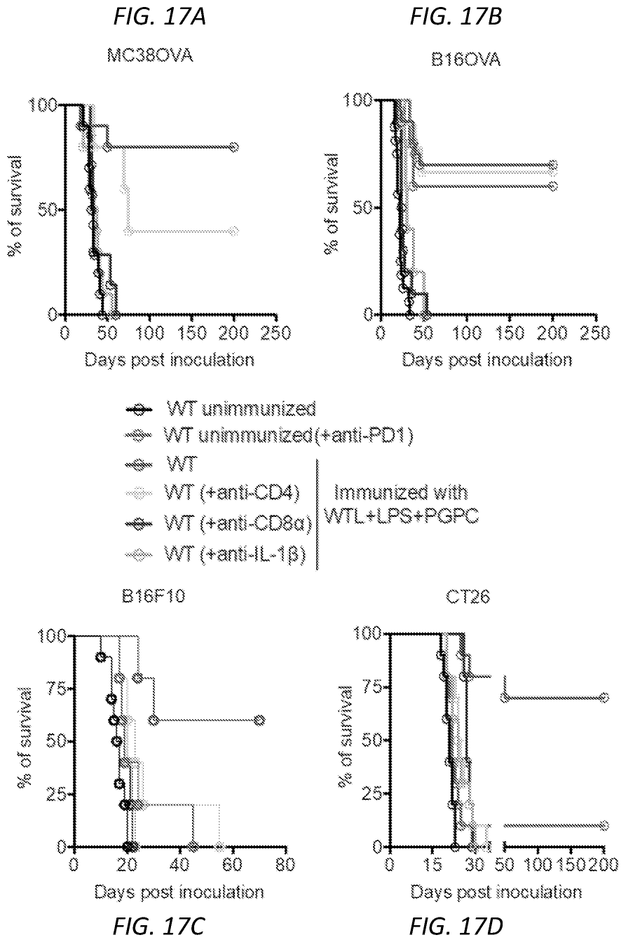

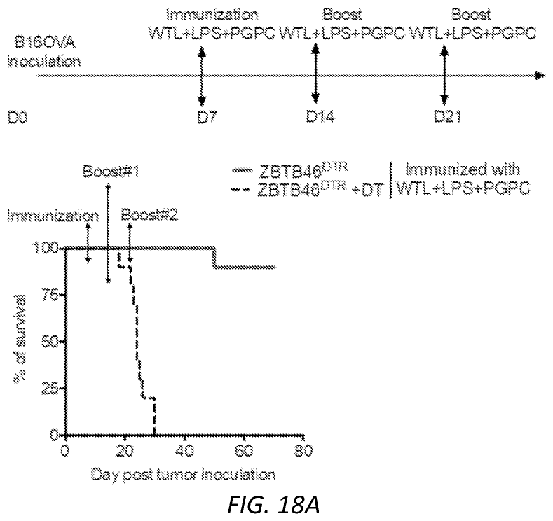

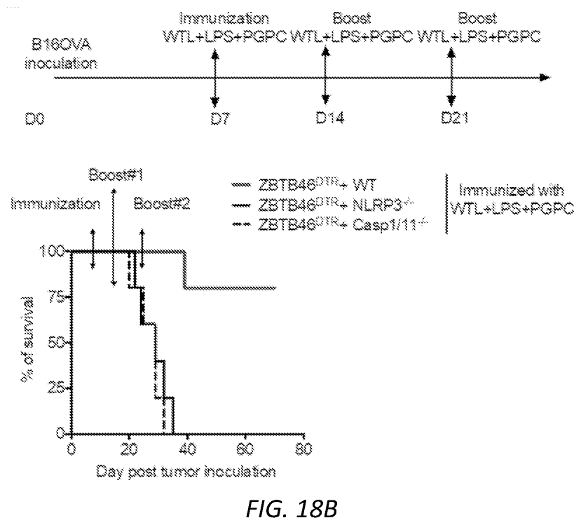

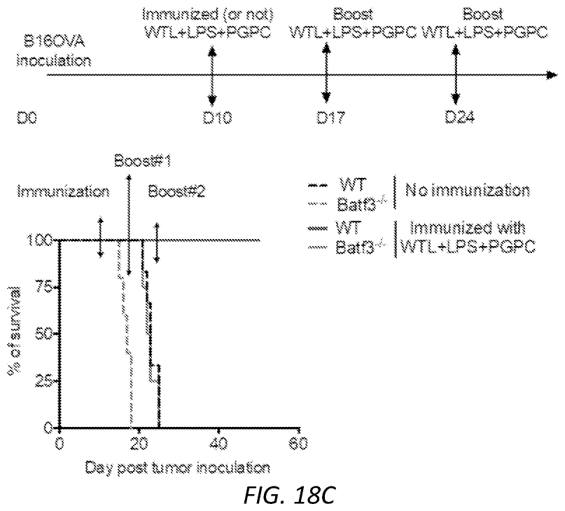

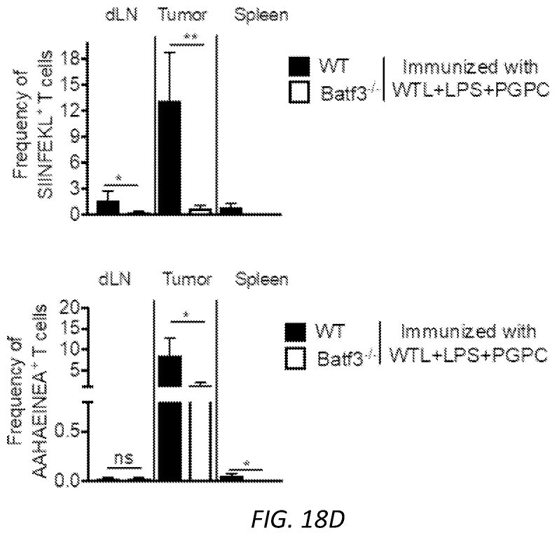

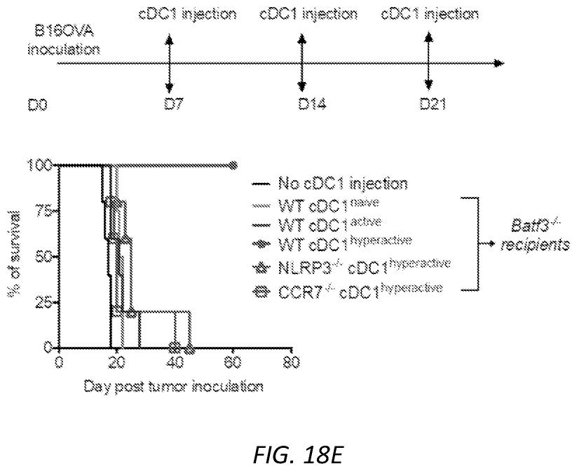

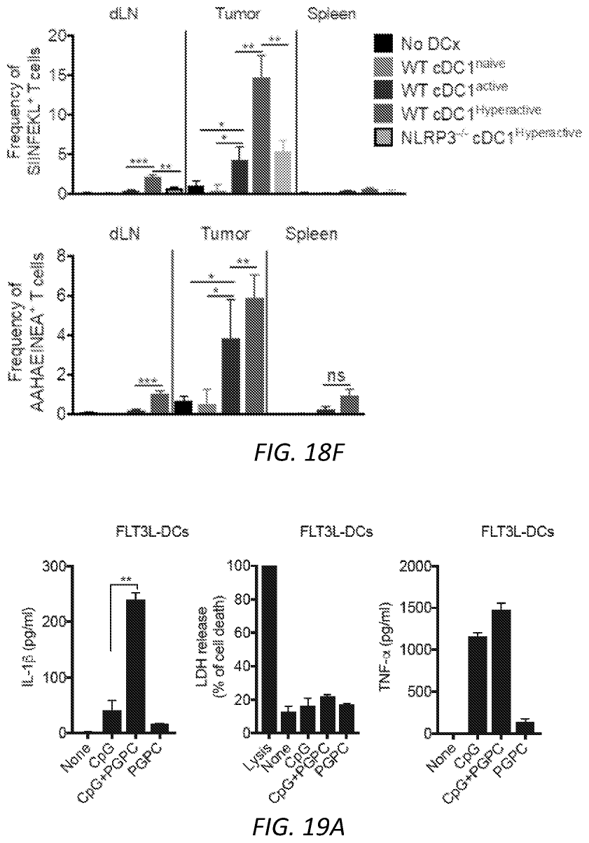

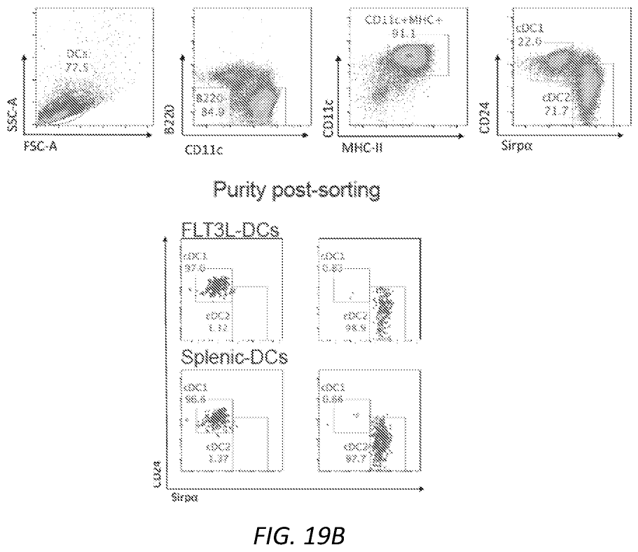

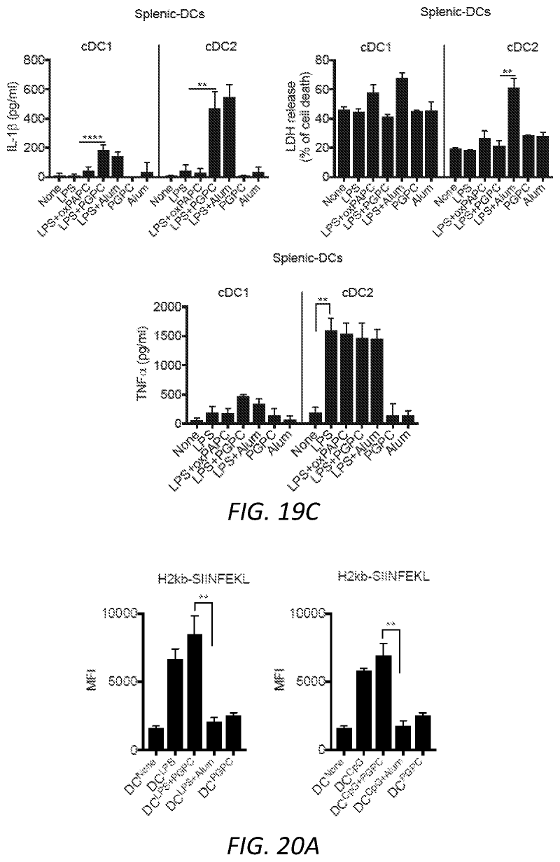

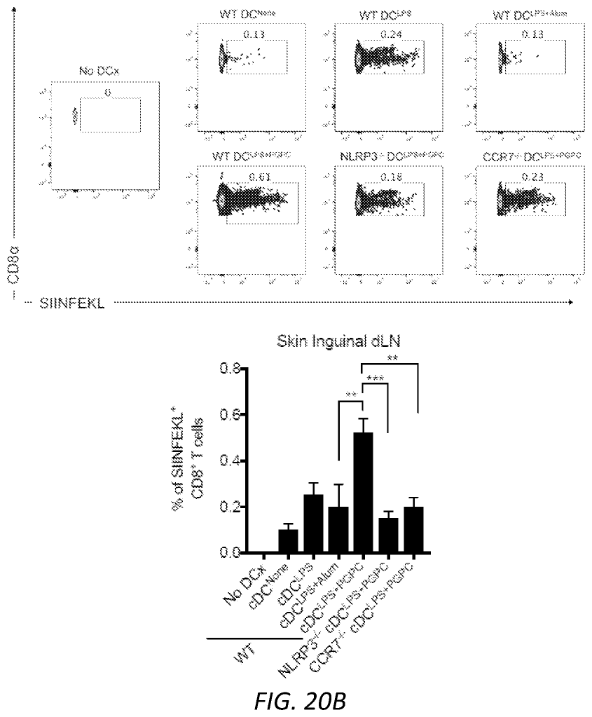

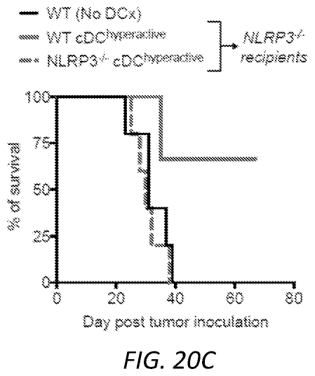

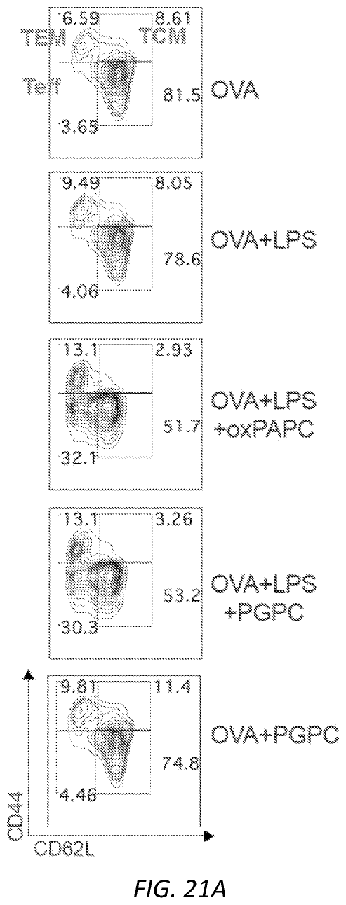

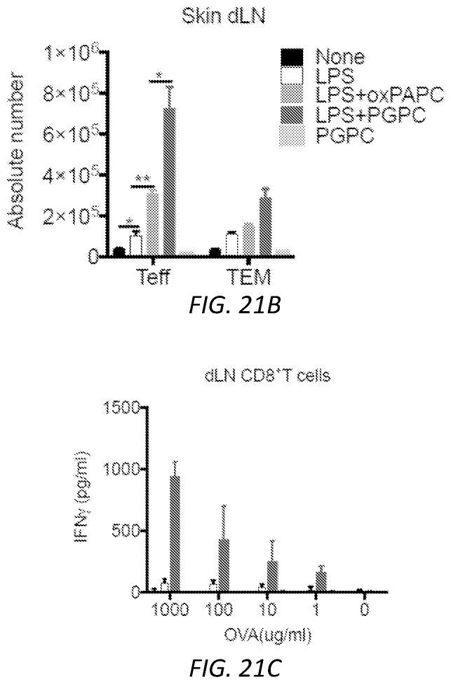

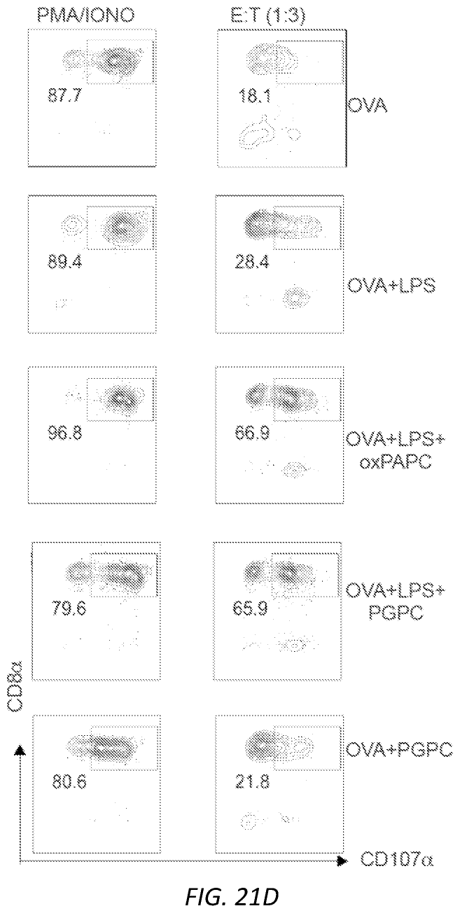

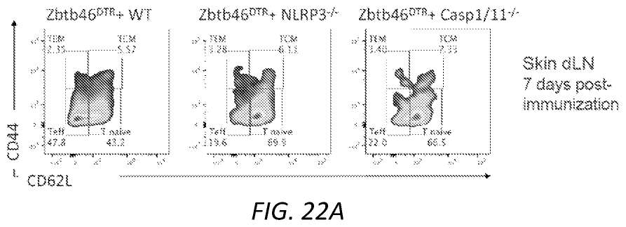

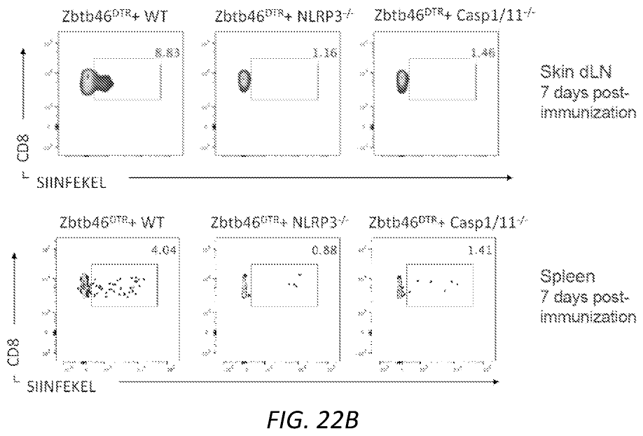

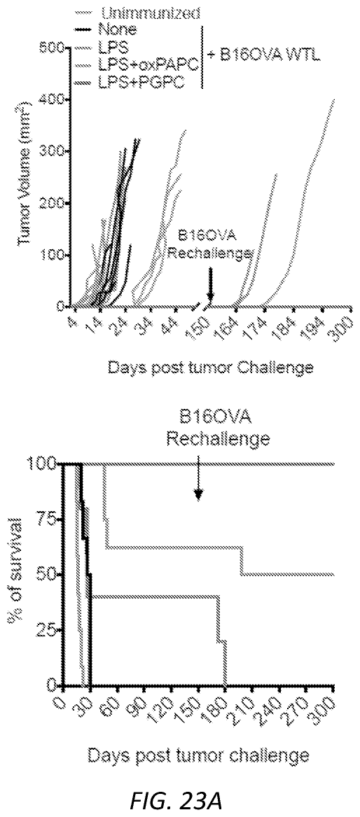

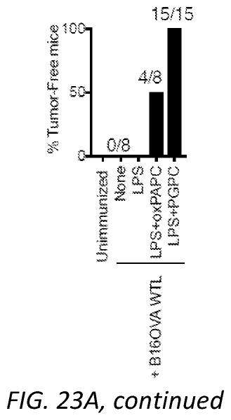

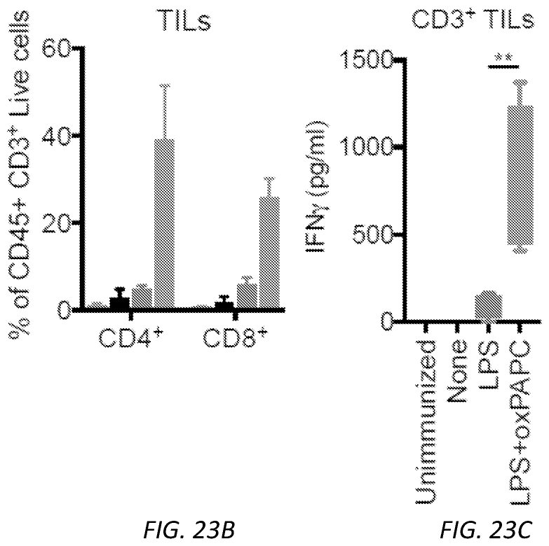

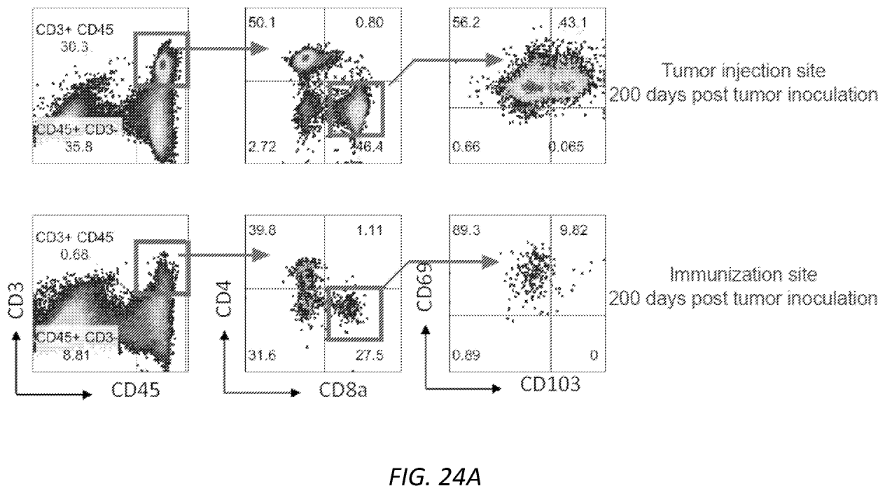

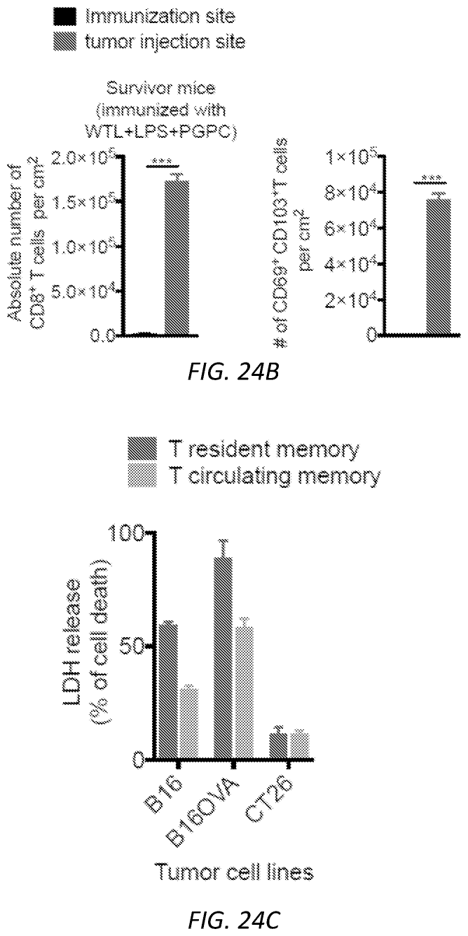

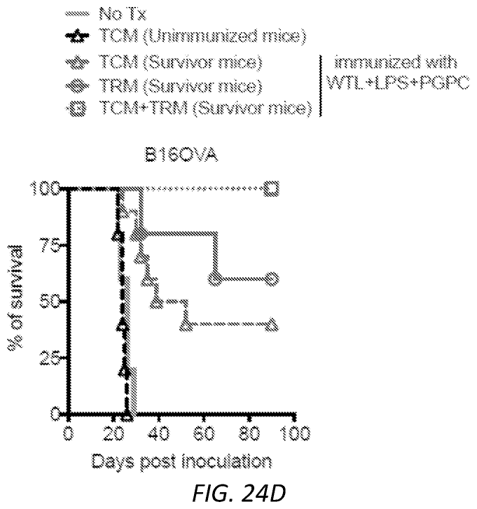

IL-1β is a critical regulator of T cell differentiation, long-lived memory T cell generation and effector function [12]-[14]. We wondered whether hyperactive DCs, which produce IL-1β over the course of several days in the dLN, can enhance CD8+ T cell stimulation. To test this, we sought to adoptively transfer DCs loaded with OVA protein subcutaneously (s.c), then measure OVA-specific CD8+ T cells in the dLN. We first tested the ability of the disparate DCs states to uptake OVA protein and to cross-present the OVA peptide SIINFEKL on H2kb molecules. We found that all DCs at the disparate states uptake OVA to a similar extend as demonstrated by the equivalent internalization of fluorescent ovalbumin (OVA-FITC). However, we found that active DCs and hyperactive DCs that were primed with LPS or with CpG both exhibited an enhanced SIINFEKL cross presentation upon OVA protein loading as compared to their naïve counterparts. This is in accordance with previous work showing that DC maturation enhances their antigen presentation capacity. Surprisingly, the pyroptotic stimuli alum strongly reduced the cross-presentation capacity of DCs suggesting that pyroptotic DCs are unfit for optimal T cell stimulation. Accordingly, when we injected 1.106 DCs of OVA-loaded naïve, active, pyroptotic or hyperactive DCs into WT mice, we observed that hyperactive DCs induced the highest frequency and absolute number of SIINFEKL+CD8+ T cells in the dLN of recipient mice (FIG. 15A and FIG. 20B) as compared to naïve, active or pyroptotic DCs. The enhanced CD8+ T cell responses mediated by hyperactive DCs was dependent on inflammasome activation since the injection of NLRP3−/− DCs treated with LPS+PGPC induced weak OVA-specific T cell responses.

### Example 5: Hyperactivating Stimuli Enhance Memory T Cell Generation and Potentiate Antigen-Specific IFNγ Effector Reponses in an Inflammasome-Dependent Manner

We hypothesized that hyperactive stimuli may represent a strong adjuvant that could recapitulate the effect of hyperactive DC injection. To examine this possibility, mice were immunized s.c. with OVA alone, or OVA plus an activating stimulus (LPS), or OVA plus a hyperactivating stimulus (LPS+oxPAPC or PGPC). 7- and 40-days post-immunization, memory and effector T cell generation in the dLN was assessed by flow cytometry using CD44 and CD62L markers that distinguish T effector cells (Teff) as CD44lowCD62Llow, T effector memory cells (TEM) as CD44hiCD62Llow, and T central memory cells (TCM) as CD44hiCD62Lhi [47]. Seven days post-immunization, hyperactivating stimuli were superior than activating stimuli at inducing CD8+ Teff cells (FIG. 16A upper panels and FIGS. 21A-21B). Furthermore, at this early time point, hyperactivating stimuli induced the highest abundance of CD8+ TEM (FIG. 16A middle panels and FIGS. 21A-21B). Forty days post-immunization, ample TCM cells were observed in mice exposed to hyperactivating stimuli, whereas these cells were less abundant in mice immunized with OVA alone or with LPS (FIG. 16A lower panels). Teff and TEM cells were conversely more abundant in mice immunized with OVA alone or with LPS as compared to mice immunized with OVA plus 40 days post immunization. Thus, these data indicate that the hyperactivating stimuli oxPAPC and PGPC enhance the magnitude of effector and memory T cell generation. Furthermore, the increase in the frequency of Teff cells 7 days post-immunization correlated with the enhanced IFNγ responses of CD8+ T cells that were isolated from the dLN of mice immunized with OVA plus hyperactivating stimuli, upon their re-stimulated ex vivo in the presence of naïve BMDCs loaded with OVA (FIG. 21C). In addition, when total CD8+ T cells were isolated from mice immunized with hyperactive stimuli and co-cultured with the B16 tumor cell line expressing OVA (B160VA), CD8+ T cells exhibited enhanced degranulation activity as compared with CD8+ T cells that were isolated from mice immunized with OVA alone or OVA plus LPS (FIG. 16B and FIG. 21D), indicating that hyperactive stimuli enhance CTLs function.

To assess the antigen-specificity of T cells that result from a s.c. immunization with the distinct activation stimuli, mice were injected with OVA, alone or with activating stimuli (LPS), or with pyroptotic stimuli (LPS+alum) or with hyperactivating stimuli (LPS+oxPAPC or PGPC). Alternatively, mice were immunized s.c. with LPS+PGPC without the OVA antigen. 7 days post immunization, CD8+ T cells were isolated from the skin dLN of immunized mice and were re-stimulated ex vivo for 7 days with naïve BMDC loaded (or not) with OVA in order to enrich the OVA-specific T cell subset. T cell effector function of OVA-specific T cells was assessed by intracellular staining for IFNγ. TCR specificity was assessed by staining with MHC-restricted OVA peptide tetramers. H2kb restricted SIINFEKL (OVA 257-264) peptide tetramers were used. The frequency of tetramer+IFNγ+ double positive cells was measured for CD4+ and CD8+ T cell subsets. Strikingly, OVA with hyperactivating stimuli were superior at inducing antigen-specific T cells, as oxPAPC- or PGPC-based immunizations resulted in the generation of the highest frequency of tetramer+IFNγ+ responses upon CD8+ T cells re-stimulation with OVA antigen (FIG. 16C). In contrast, pyroptotic stimuli (LPS+alum) were the weakest inducers of antigen-specific IFNγ responses (FIG. 16C). These results are in accordance with previous studies which indicated that alum is an adjuvant that is effective for promoting humoral immunity and Th2 responses, but not Th1 or CTL responses [39,42,43].

Previous studies showed that antigen-specific T cell responses can be enhanced by co-immunization with recombinant IL-1β [47,13], a cytokine whose bioactivity is naturally controlled by inflammasomes. However, despite the fact that both hyperactive and pyroptotic stimuli induce IL-1β secretion, how hyperactive stimuli but not pyroptotic stimuli induce higher antigen-specific T cells? To determine if inflammasome-mediated events control the T cell responses generated with hyperactivating stimuli, side-by-side comparisons of T cell activity were performed in WT and NLRP3−/− mice. Notably, we found that NLRP3 was required for the hyperactivation-induced enhancement of antigen-specific responses by CD8+T cells (FIG. 16C). Thus these data indicate that non-pyroptotic vs pyroptotic inflammasome activation following immunization with hyperactivating stimuli or pyroptotic stimuli respectively, induce a striking differential adaptive immune T cell control.

Our previous results using DC injection strategies indicate that DCs stimulated with pyroptotic stimuli lose their ability to migrate to adjacent dLN and to stimulate T cell activation, whereas DCs exposed to hyperactive stimuli hypermigrate to dLN and potentiate CTL responses (FIGS. 15A-15B). However, Its unknown whether endogenous DCs can achieve hyperactivation in vivo following immunization with hyperactive stimuli. To assess this, we generated mouse chimeras using Zbtb46DTR and WT mice or Zbtb46DTR and NLRP3−/− mice or Zbtb46DTR and Casp1/11−/− mice. To this end, 4 weeks old CD45.1 irradiated mice were reconstituted with mixed bone marrow of 80% of Zbtb46DTR and 20% of WT mice or 20% of NLRP3−/− or 20% of Casp1/11−/− mice on a CD45.2 background as previously described[51]. Six-weeks post-reconstitution, the efficacy of BM reconstitution in all mice was assessed by flow cytometry using cD45.1 vs CD45.2 markers. Chimera mice were treated every other day with diptheria toxin (DT) to deplete Zbtb46+ conventional DCs, giving rise to mice that harbor either WT or inflammasome deficient (NLRP3−/− or Casp1/11−/−) DCs which can or cannot get hyperactive respectively. To test the effect of endogenous DC-hyperactivation on CD8+ T cell responses, all chimera mice were s.c. immunized with OVA plus LPS+PGPC following 3 consecutive DT injections. 7 days post-immunization, CD8+ T cell response from the dLN was assessed. Interestingly, we found that the abundance of Teff CD8+ T cells was strongly reduced in the chimera mice harboring DCs that cannot get hyperactive such as in NLRP3−/− and Casp1/11−/− chimeras mice, as compared to the chimera mice harboring WT DCs that can get hyperactive (FIG. 16D, FIG. 22A). Furthermore, we discovered that the frequency of SIINFEKL+CD8+ T cells in the dLN or the spleen was reduced in NLRP3−/− and Casp1/11−/− chimera mice harboring DCs that cannot get hyperactive, whereas high SIINFEKL+CD8+ cells were observed in chimera harboring WT DCs (FIG. 16E, FIG. 22B). Thus, these data clearly show that: 1) endogenous DCs can achieve a state of hyperactivation in vivo, and potentiate CTLs responses following immunization with hyperactivating stimuli, and 2) inflammasome activation within endogenous DCs is crucial for hyperactivation-mediated protective CTL responses.

### Example 6: Hyperactive DC Entry to Lymphoid Tissues is Essential for Hyperactivation-Mediated CTLs Responses

We previously showed that DCs stimulated with hyperactive stimuli hypermigrate to dLN and potentiate CTL responses (FIGS. 15A-15B). To assess if hyperactivation-mediated CTLs responses require the entry of endogenous hyperactive DCs to dLN, we generated mice chimeras as described above, using Zbtb46DTR and WT or Zbtb46DTR and CCR7−/− BM (FIG. 15D). Overall, endogenous trafficking of hyperactive DCs to dLN is essential for hyperactivation-mediated CTLs function.

### Example 7: Hyperactive Stimuli can Use Complex Antigen Sources to Stimulate Prophylactic T Cell Mediated Anti-Tumor Immunity

Current efforts to stimulate anti-tumor immunity include strategies that invigorate resident T cell populations (e.g. PD-1 blockade) or personalized cancer vaccine strategies that stimulate the generation of de novo T cell responses to tumor-specific antigens (TSAs) [46]. This latter effort has been hampered by the inability to use tumor cell lysates as a source of TSAs, also known as neo-antigens. Consequently, efforts are underway to improve the identification of neo-antigens, which can be used in pure form to elicit T cell mediated anti-tumor immunity. While these efforts have yielded successes [50,49,51], the path to neo-antigen identification requires a pipeline for mutated, as well as aberrantly expressed TSAs discovery [54], which is laborious and not representative of the natural course of events. As previously discussed, WTL represent an attractive alternative source of antigens, as these lysates provide a large number of antigens that are required for the initiation of a personalized anti-tumor immune responses. However, fundamental questions such as what is the most effective type of adjuvant to be used in cancer vaccines (including the type of adjuvant to be associated with different type of antigens) still remain unanswered.

To address the possibility that hyperactivating stimuli can be an adjuvant to WTL, mice were immunized on the right flank with WTL alone, or WTL mixed with the activating stimulus LPS or the hyperactivating stimuli LPS+oxPAPC or LPS+PGPC. The source of the WTL was B160VA cells. Fifteen days post-immunization, mice were challenged s.c on the left upper back with the parental B16OVA cells. Unimmunized mice or mice immunized with WTL alone did not exhibit any protection, and all mice harbored large tumors by day 24 after tumor inoculation and died (FIG. 23A). Similarly, WTL+LPS immunizations offered minimal protection. Two of eight mice immunized with WTL+LPS were tumor-free, but quickly relapsed after B160VA re-challenge (FIG. 23A), indicating no protective immunity was conferred by stimuli that merely activate DCs. In contrast, WTL immunizations in the presence of LPS and oxPAPC induced a significant delay in the tumor growth and resulted in a strong protection against subsequent lethal re-challenge with parental B160VA tumor cells; 50% of the immunized mice were fully protected (FIG. 23A). To determine if the protective responses induced by oxPAPC correlated with T cell responses, tumors were harvested from mice receiving each activation stimulus. Tumors from mice immunized with LPS+oxPAPC contained a substantial abundance of CD4+ and CD8+ T cells, as compared to LPS immunizations (Figure S7B). Moreover, when equal numbers of T cells from these tumors were compared, oxPAPC-based immunizations resulted in intra-tumoral T cells that secreted the highest amounts of IFNγ upon anti-CD3 and anti-CD28 stimulation (FIG. 23C). Thus, the superior restriction of tumor growth induced by hyperactivating stimuli (LPS+oxPAPC) was coincident with inflammatory T cell infiltration into the tumor.

Notably, the protective phenotypes of oxPAPC were superseded by those elicited by the pure oxPAPC component PGPC. WTL immunizations in the presence of LPS+PGPC led to 100% of mice being tumor-free for 150 days post-tumor challenge. These mice completely rejected a lethal re-challenge with B160VA cells and remained tumor-free 300 days post initial tumor challenge (FIG. 20A). Since these mice never relapsed, we wondered how tumor cell growth is kept under control at the site of tumor injection in mice immunized with WTL plus LPS+PGPC?

Among memory T cell subsets, T resident memory cells (TRM) are defined by the expression of CD103 integrin along with C-type lectin CD69, which contribute to their residency characteristic in the peripheral tissues [55]. CD8+ TRM cells have recently gained much attention, as these cells accumulate at the tumor site in various human cancer tissues and correlate with the more favorable clinical outcome [54,55,56]. In an experimental cutaneous melanoma model, CD8+ TRM cells in the skin promoted durable protection against melanoma progression [58].

We examined the presence of TRM cells at the tumor injection site, as well as the immunization skin biopsies in the survivor mice that were previously immunized with the hyperactivating stimuli LPS+PGPC. Interestingly, 200 days post-tumor inoculation, CD8+CD69+CD103+ TRM cells were highly enriched at the site of tumor injection but were scarce at the immunization site in all survivor mice (FIGS. 24A-24B). These data are in accordance with clinical and experimental reports that correlate high levels of TRM with a long-term tumor control, in which TRM are potentially maintained for long periods to survey tumor injection site [56,57]. Thus, it is likely that the immunization with WTL and the hyperactivating stimuli generate TRM that keeps tumor cells in check.

To examine the functional specificity of these T cells, we monitored cytotoxic lymphocyte (CTL) activity ex vivo. Circulating memory CD8+ T cells and TRM cells were isolated from the spleen or the skin adipose tissue of survivor mice that previously received hyperactivating stimuli. These cells were cultured with B16OVA cells, or B16 cells not expressing OVA or an unrelated cancer cell line CT26. CTL activity, as assessed by LDH release, was only observed when CD8+ T cells were mixed with B160VA or B16 cells (FIG. 24C). No killing of CT26 cells was observed (FIG. 24C), thus indicating the functional and antigen-specific nature of hyperactivation-induced T cell responses.

Based on the antigen-specific T cell responses induced by hyperactivating stimuli, we determined if T cells are sufficient to protect against tumor progression. CD8+ T cells were transferred from survivor mice into naïve mice and subsequently challenged with the parental tumor cell line used as the initial immunogen. Transfer of CD8+ TRM or circulating CD8+ T cells from survivor mice into naïve recipients conferred profound protection from a subsequent tumor challenge, with the TRM subset playing a dominant protective role (FIG. 24D). Transfer of both T cell subsets from survivor mice into naïve mice, one week before tumor inoculation, provided 100% protection of recipient mice from subsequent tumor challenges (FIG. 24D). These collective data indicate that PGPC-based hyperactivation stimuli confer optimal protection in a B16 melanoma model by inducing strong circulating and resident anti-tumor CD8+ T cell responses.

### Example 8: Hyperactive Stimuli Protect Against Established Anti-PD1 Resistant Tumors

To determine if hyperactivating stimuli could be harnessed as a cancer immunotherapy, we examined anti-tumor responses in mice that harbored a growing tumor prior to any additional treatment. For these studies, rather than using cultured tumor cells as an antigen source, ex vivo WTL were generated using syngeneic tumors from unimmunized mice, in which 10 mm harvested tumors were dissociated and then depleted of CD45+ cells. Mice were inoculated subcutaneously (s.c.) with tumor cells on the left upper back. When tumors reached a size of 3-4 mm, tumor-bearing mice were either left untreated (unimmunized) or received a therapeutic injection on the right flank, which consisted of ex vivo WTL and LPS+PGPC. Two subsequent s.c. boosts of therapeutic injections were performed (FIG. 17A). Interestingly, hyperactivation-based therapeutic injections induced tumor eradication in a wide range of tumors such as B16OVA and B16F10 melanoma models, in MC38OVA and CT26 colon cancer tumor models (FIGS. 17B-17D). In all of these models, a high percentage of mice that received the immunotherapy regimen remained tumor-free long after the tumor inoculation (FIGS. 17B-17D). The efficacy of the immunotherapy was dependent on IL-1β in all the tested tumor models, since the neutralization of IL-1β abolished protection conferred by hyperactivating stimuli plus ex vivo WTL (FIGS. 17B-17D). In addition, CD8+ T cells were crucial for protection against immunogenic tumor models such as B16OVA or MC380VA tumors, whereas CD4+ and CD8+ T cells were both required for protection against less immunogenic tumors such as CT26, and B16F-10 (FIGS. 17B-17D) [63]. To determine how hyperactivation-based immunotherapy compare in efficacy to therapies based on PD-1 blockade, side-by-side assessments were performed. Hyperactivation-based immunotherapy was as efficient as anti-PD-1 therapy in the immunogenic B16OVA model, but more efficient in tumor models that are insensitive to anti-PD-1 treatment such as CT26, and B16F-10 (FIGS. 17B-17D).

### Example 9: Endogenous Hyperactive DCs Stimulate T Cell Mediated Durable Anti-Tumor Immunity

The adoptive transfer of hyperactive DCs into tumor-bearing mice induce strong anti-tumor immunity (FIGS. 15A-15B). To test whether endogenous DCs can initiate hyperactivation-mediated anti-tumor response, we used Zbtb46DTR mice in which conventional DCs are depleted by DT injection. Zbtb46DTR or WT mice were injected s.c. with B16OVA cells. DT was then injected every other day to fully deplete resident DCs in Zbtb46DTR mice prior to their immunization. When tumors reached 4 mm of size, Zbtb46DTR or WT mice were immunized with B16OVA WTL plus the hyperactivating stimuli LPS+PGPC. We found that in contrast to WT mice, which rejected tumors in 90% of mice, Zbtb46DTR mice (which lack DCs) were unable to reject tumors. These data confirm that DCs are the initiator of hyperactivation-mediated protection (FIG. 18A).

### Example 10: Hyperactive cDC1 can Use Complex Antigen Sources to Stimulate T Cell Mediated Anti-Tumor Immunity

We demonstrated in vitro that cDC1 and cDC2 both can achieve a state of hyperactivation, as these cells produce IL-1β in response to LPS+PGPC while maintaining their viability. These data provide the mandate to further define the specific DC subset that initiate hyperactivation-mediated anti-tumor response in vivo. Given the importance of cDC1 subset in tumor rejection, we hypothesized that cDC1 play a central role in inducing hyperactivation-mediated anti-tumor protection. To test this idea, we used Batf3−/− mice (which lack cDC1, but harbor cDC2 cells) [65]. To this end, we immunized Batf3−/− or WT tumor-bearing mice (harboring a B16OVA tumor of 3-4 mm) with LPS+PGPC and WTL. These mice received two s.c boost injections every 7 days. We observed that unimmunized Batf3−/− mice displayed a more severe tumor growth as unimmunized WT mice, and all mice succumb to tumors as soon as 18 days post tumor inoculation. This data corroborate with previous studies showing that the rejection of highly immunogenic tumors is strongly impaired in Batf3−/− mice that lack cDC1 cells [65]. Interestingly, despite the fact that the immunization of Batf3−/− mice improved their survival by few days as compared to unimmunized Batf3−/− mice, all Batf3−/− mice succumbed to tumor growth by 25 days post tumor inoculation. In contrast, WT mice rejected tumors in 100% of tumor-bearing mice (FIG. 18C). Thus, cDC1 play a critical role in hyperactivation-mediate anti-tumor immunity. In addition, while immunized WT mice induced high frequency of antigen-specific CD8+ and CD4+ T cells in the TME and in the skin dLN, immunized Batf3−/− induced slightly reduced antigen CD4+ T cells, but strikingly no antigen-specific CD8+ T cells in the TME (FIG. 18D).

To further confirm the role of hyperactive cDC1 in inducing long-live anti-tumor protection, we sought to assess the ability of hyperactive cDC1 to restore anti-tumor protection in Batf3−/−. We adoptively transferred naïve, active, or hyperactive cDC1 cells into Batf3−/− mice. To this end, FLT3-derived cDC1 were sorted from C57BL/6J mice as B220-MHC-II+CD11c+CD24+ cells as previously described. cDC1 were treated as described above in vitro and loaded with B16OVA WTL, then 1.10e6 cells were injected s.c. into tumor-bearing Batf3−/− mice. We observed that in contrast to naïve, or active cDC1 which provided only a slight improved mice survival as compared to uninjected mice, hyperactive cDC1 induced tumor rejection in 100% of tumor-bearing mice, which remained tumor-free for more than 60 days post tumor inoculation (FIG. 18E). Of note, hyperactive cDC1 injection restored CD8+ T cell responses in Batf3−/− as measured by SIINFEKL tetramer staining in the tumor and in the skin dLN (FIG. 18F). In contrast, naïve or active cDC1 injection failed to restore antigen-specific CD8+ T cells. cDC1 mediated tumor rejection was dependent on inflammasome activation, since the injection of NLRP3−/− cDC1 that were treated with LPS+PGPC did not provide any anti-tumor protection, and abrogated the ability of hyperactive cCD1 to restore CD8+ T cells responses (FIG. 18F).

In addition to their ability to produce IL-1 from living cells, hyperactive DCs highly migrate to adjacent dLN to potentiate CD8+ T cell responses (FIGS. 15A-15B).

### REFERENCES FOR EXAMPLES 3-10

[1] D. Alvarez, E. H. Vollmann, and U. H. von Andrian, “Mechanisms and consequences of dendritic cell migration.,” Immunity, vol. 29, no. 3, pp. 325-42, September 2008.
[2] S. W. Brubaker, K. S. Bonham, I. Zanoni, and J. C. Kagan, “Innate immune pattern recognition: a cell biological perspective.,” Annu. Rev. Immunol., vol. 33, pp. 257-90, 2015.
[3] C. A. Janeway and R. Medzhitov, “Innate immune recognition.,” Annu. Rev. Immunol., vol. 20, pp. 197-216, January 2002.
[4] P. Matzinger, “The danger model: a renewed sense of self.,” Science, vol. 296, no. 5566, pp. 301-5, April 2002.
[5] A. Iwasaki and R. Medzhitov, “Control of adaptive immunity by the innate immune system.,” Nat. Immunol., vol. 16, no. 4, pp. 343-53, April 2015.
[6] O. Joffre, M. A. Nolte, R. Spörri, and C. R. e Sousa, “Inflammatory signals in dendritic cell activation and the induction of adaptive immunity,” Immunol. Rev., vol. 227, no. 1, pp. 234-247, January 2009.
[7] K. Inaba et al., “The formation of immunogenic major histocompatibility complex class II-peptide ligands in lysosomal compartments of dendritic cells is regulated by inflammatory stimuli.,” J. Exp. Med., vol. 191, no. 6, pp. 927-36, March 2000.
[8] I. Mellman and R. M. Steinman, “Dendritic cells: specialized and regulated antigen processing machines.,” Cell, vol. 106, no. 3, pp. 255-8, August 2001.
[9] J. Paavonen et al., “Efficacy of human papillomavirus (HPV)-16/18 AS04-adjuvanted vaccine against cervical infection and precancer caused by oncogenic HPV types (PATRICIA): final analysis of a double-blind, randomised study in young women,” Lancet, vol. 374, no. 9686, pp. 301-314, July 2009.
[10] M. Kundi, “New hepatitis B vaccine formulated with an improved adjuvant system,” Expert Rev. Vaccines, vol. 6, no. 2, pp. 133-140, April 2007.
[11] A. M. Didierlaurent et al., “AS04, an Aluminum Salt- and TLR4 Agonist-Based Adjuvant System, Induces a Transient Localized Innate Immune Response Leading to Enhanced Adaptive Immunity,” J. Immunol., vol. 183, no. 10, pp. 6186-6197, November 2009.
[12] S. Z. Ben-Sasson et al., “IL-1 acts directly on CD4 T cells to enhance their antigen-driven expansion and differentiation.,” Proc. Natl. Acad. Sci. U.S.A, vol. 106, no. 17, pp. 7119-24, April 2009.
[13] S. Z. Ben-Sasson et al., “IL-1 enhances expansion, effector function, tissue localization, and memory response of antigen-specific CD8 T cells.,” J. Exp. Med., vol. 210, no. 3, pp. 491-502, March 2013.
[14] A. Jain, R. Song, E. K. Wakeland, and C. Pasare, “T cell-intrinsic IL-1R signaling licenses effector cytokine production by memory CD4 T cells,” Nat. Commun., vol. 9, no. 1, pp. 1-13, 2018.
[15] C. Garlanda, C. A. Dinarello, and A. Mantovani, “The Interleukin-1 Family: Back to the Future,” Immunity, vol. 39, no. 6, pp. 1003-1018, December 2013.
[16] A. Lu et al., “Unified polymerization mechanism for the assembly of ASC-dependent inflammasomes.,” Cell, vol. 156, no. 6, pp. 1193-1206, March 2014.
[17] J. C. Kagan, V. G. Magupalli, and H. Wu, “SMOCs: supramolecular organizing centres that control innate immunity,” Nat. Rev. Immunol., vol. 14, no. 12, pp. 821-826, December 2014.
[18] K. J. Kieser and J. C. Kagan, “Multi-receptor detection of individual bacterial products by the innate immune system,” Nat. Rev. Immunol., vol. 17, no. 6, pp. 376-390, May 2017.
[19] M. Lamkanfi and V. M. Dixit, “Mechanisms and functions of inflammasomes.,” Cell, vol. 157, no. 5, pp. 1013-22, May 2014.
[20] T. R. Mempel, S. E. Henrickson, and U. H. von Andrian, “T-cell priming by dendritic cells in lymph nodes occurs in three distinct phases,” Nature, vol. 427, no. 6970, pp. 154-159, January 2004.
[21] S. C. Eisenbarth, O. R. Colegio, W. O'Connor, F. S. Sutterwala, and R. A. Flavell, “Crucial role for the Nalp3 inflammasome in the immunostimulatory properties of aluminium adjuvants,” Nature, vol. 453, no. 7198, pp. 1122-1126, June 2008.
[22] M. Kool et al., “Cutting Edge: Alum Adjuvant Stimulates Inflammatory Dendritic Cells through Activation of the NALP3 Inflammasome,” J. Immunol., vol. 181, no. 6, pp. 3755-3759, September 2008.
[23] P. Marrack, A. S. McKee, and M. W. Munks, “Towards an understanding of the adjuvant action of aluminium,” Nat. Rev. Immunol., vol. 9, no. 4, pp. 287-293, April 2009.
[24] I. Zanoni et al., “An endogenous caspase-II ligand elicits interleukin-1 release from living dendritic cells,” Science (80-)., vol. 352, no. 6290, pp. 1232-1236, 2016.
[25] I. Zanoni, Y. Tan, M. Di Gioia, J. R. Springstead, and J. C. Kagan, “By Capturing Inflammatory Lipids Released from Dying Cells, the Receptor CD14 Induces Inflammasome-Dependent Phagocyte Hyperactivation.,” Immunity, vol. 47, no. 4, pp. 697-709.e3, 2017.
[26] C. L. Evavold, J. Ruan, Y. Tan, S. Xia, H. Wu, and J. C. Kagan, “The Pore-Forming Protein Gasdermin D Regulates Interleukin-1 Secretion from Living Macrophages.,” Immunity, vol. 0, no. 0, November 2017.
[27] X. Liu et al., “Inflammasome-activated gasdermin D causes pyroptosis by forming membrane pores,” Nature, vol. 535, no. 7610, pp. 153-158, 2016.
[28] R. A. Aglietti et al., “GsdmD p30 elicited by caspase-11 during pyroptosis forms pores in membranes.,” Proc. Natl. Acad. Sci. U.S.A, vol. 113, no. 28, pp. 7858-63, July 2016.
[29] N. Kayagaki et al., “Caspase-11 cleaves gasdermin D for non-canonical inflammasome signalling,” Nature, vol. 526, no. 7575, pp. 666-671, September 2015.
[30] S. Rühl, K. Shkarina, B. Demarco, R. Heilig, J. C. Santos, and P. Broz, “ESCRT—dependent membrane repair negatively regulates pyroptosis downstream of GSDMD activation.,” Science, vol. 362, no. 6417, pp. 956-960, November 2018.
[31] M. M. Gaidt et al., “Human Monocytes Engage an Alternative Inflammasome Pathway,”
Immunity, vol. 44, no. 4, pp. 833-846, April 2016.
[32] A. J. Wolf et al., “Hexokinase Is an Innate Immune Receptor for the Detection of Bacterial Peptidoglycan,” Cell, vol. 166, no. 3, pp. 624-636, July 2016.
[33] M. Monteleone et al., “Interleukin-10 Maturation Triggers Its Relocation to the Plasma Membrane for Gasdermin-D-Dependent and -Independent Secretion,” Cell Rep., vol. 24, no. 6, pp. 1425-1433, August 2018.
[34] K. W. Chen et al., “The Neutrophil NLRC4 Inflammasome Selectively Promotes IL-1β Maturation without Pyroptosis during Acute Salmonella Challenge,” Cell Rep., vol. 8, no. 2, pp. 570-582, July 2014.
[35] Z. Erlich et al., “Macrophages, rather than DCs, are responsible for inflammasome activity in the GM-CSF BMDC model,” Nat. Immunol.
[36] I. Zanoni et al., “An endogenous caspase-II ligand elicits interleukin-1 release from living dendritic cells,” Science (80-)., vol. 352, no. 6290, pp. 1232-1236, June 2016.
[37] J.-C. Cancel, K. Crozat, M. Dalod, and R. Mattiuz, “Are Conventional Type 1 Dendritic Cells Critical for Protective Antitumor Immunity and How?,” Front. Immunol., vol. 10, p. 9, February 2019.
[38] D. J. Theisen et al., “Batf3-Dependent Genes Control Tumor Rejection Induced by Dendritic Cells Independently of Cross-Presentation.,” Cancer Immunol. Res., vol. 7, no. 1, pp. 29-39, January 2019.
[39] I. Dang et al., “Inhibitory signalling to the Arp2/3 complex steers cell migration,” Nature, vol. 503, no. 7475, pp. 281-284, November 2013.
[40] K. A. Hogquist, S. C. Jameson, W. R. Heath, J. L. Howard, M. J. Bevan, and F. R. Carbone, “T cell receptor antagonist peptides induce positive selection,” Cell, vol. 76, no. 1, pp. 17-27, January 1994.
[41] M. J. Bamden, J. Allison, W. R. Heath, and F. R. Carbone, “Defective TCR expression in transgenic mice constructed using cDNA-based α- and β-chain genes under the control of heterologous regulatory elements,” Immunol. Cell Biol., vol. 76, no. 1, pp. 34-40, February 1998.
[42] E. Oleszycka et al., “The vaccine adjuvant alum promotes IL-10 production that suppresses Th1 responses,” Eur. J. Immunol., vol. 48, no. 4, pp. 705-715, April 2018.
[43] M. J. Newman et al., “Saponin adjuvant induction of ovalbumin-specific CD8+ cytotoxic T lymphocyte responses.,” J. Immunol., vol. 148, no. 8, pp. 2357-62, April 1992.
[44] R. L. Sabado, S. Balan, and N. Bhardwaj, “Dendritic cell-based immunotherapy.,” Cell Res., vol. 27, no. 1, pp. 74-95, January 2017.
[45] A. Draube et al., “Dendritic Cell Based Tumor Vaccination in Prostate and Renal Cell Cancer: A Systematic Review and Meta-Analysis,” PLoS One, vol. 6, no. 4, p. e18801, April 2011.
[46] Z. Hu, P. A. Ott, and C. J. Wu, “Towards personalized, tumour-specific, therapeutic vaccines for cancer,” Nat. Rev. Immunol., vol. 18, no. 3, pp. 168-182, December 2017.
[47] F. Sallusto, D. Lenig, R. Forster, M. Lipp, and A. Lanzavecchia, “Two subsets of memory T lymphocytes with distinct homing potentials and effector functions,” Nature, vol. 401, no. 6754, pp. 708-712, October 1999.
[48] J. M. Brewer, M. Conacher, C. A. Hunter, M. Mohrs, F. Brombacher, and J. Alexander, “Aluminium hydroxide adjuvant initiates strong antigen-specific Th2 responses in the absence of IL-4- or IL-13-mediated signaling.,” J. Immunol., vol. 163, no. 12, pp. 6448-54, December 1999.
[49] A. Mori et al., “The vaccine adjuvant alum inhibits IL-12 by promoting PI3 kinase signaling while chitosan does not inhibit IL-12 and enhances Th1 and Th17 responses,” Eur. J. Immunol., vol. 42, no. 10, pp. 2709-2719, October 2012.
[50] S. Z. Ben-Sasson, K. Wang, J. Cohen, and W. E. Paul, “IL-1 Strikingly Enhances Antigen-Driven CD4 and CD8 T-Cell Responses,” Cold Spring Harb. Symp. Quant. Biol., vol. 78, no. 0, pp. 117-124, January 2013.
[51] I. Zanoni et al., “IL-15 cis Presentation Is Required for Optimal NK Cell Activation in Lipopolysaccharide-Mediated Inflammatory Conditions,” Cell Rep., vol. 4, no. 6, pp. 1235-1249, 2013.
[52] D. B. Keskin et al., “Neoantigen vaccine generates intratumoral T cell responses in phase Ib glioblastoma trial,” Nature, vol. 565, no. 7738, pp. 234-239, January 2019.
[53] P. A. Ott et al., “An immunogenic personal neoantigen vaccine for patients with melanoma.,” Nature, vol. 547, no. 7662, pp. 217-221, 2017.
[54] C. M. Laumont et al., “Noncoding regions are the main source of targetable tumor-specific antigens.,” Sci. Transl. Med., vol. 10, no. 470, p. eaau5516, December 2018.
[55] F. Mami-Chouaib et al., “Resident memory T cells, critical components in tumor immunology,” J. Immunother. Cancer, vol. 6, no. 1, p. 87, December 2018.
[56] J. R. Webb, K. Milne, P. Watson, R. J. deLeeuw, and B. H. Nelson, “Tumor-Infiltrating Lymphocytes Expressing the Tissue Resident Memory Marker CD103 Are Associated with Increased Survival in High-Grade Serous Ovarian Cancer,” Clin. Cancer Res., vol. 20, no. 2, pp. 434-444, January 2014.
[57] F. Djenidi et al., “CD8+CD103+ tumor-infiltrating lymphocytes are tumor-specific tissue-resident memory T cells and a prognostic factor for survival in lung cancer patients.,” J. Immunol., vol. 194, no. 7, pp. 3475-86, April 2015.
[58] S. L. Park et al., “Tissue-resident memory CD8+ T cells promote melanoma-immune equilibrium in skin,” Nature, vol. 565, no. 7739, pp. 366-371, January 2019.
[59] C. M. Koebel et al., “Adaptive immunity maintains occult cancer in an equilibrium state,” Nature, vol. 450, no. 7171, pp. 903-907, December 2007.
[60] S.-J. Han et al., “White Adipose Tissue Is a Reservoir for Memory T Cells and Promotes Protective Memory Response0073 to Infection.,” Immunity, vol. 47, no. 6, pp. 1154-1168.e6, 2017.
[61] J. C. Castle et al., “Exploiting the mutanome for tumor vaccination.,” Cancer Res., vol. 72, no. 5, pp. 1081-91, March 2012.
[62] M. O. Mohsen et al., “Targeting Mutated Plus Germline Epitopes Confers Pre-clinical Efficacy of an Instantly Formulated Cancer Nano-Vaccine.,” Front. Immunol., vol. 10, p. 1015, 2019.
[63] S. I. S. Mosely et al., “Rational Selection of Syngeneic Preclinical Tumor Models for Immunotherapeutic Drug Discovery,” 2017.
[64] M. G. Lechner et al., “Immunogenicity of murine solid tumor models as a defining feature of in vivo behavior and response to immunotherapy,” J. Immunother., vol. 36, no. 9, pp. 477-489, November 2013.
[65] K. Hildner et al., “Batf3 deficiency reveals a critical role for CD8α+ dendritic cells in cytotoxic T cell immunity,” Science (80-)., vol. 322, no. 5904, pp. 1097-1100, November 2008.
[66] M. Kool et al., “Alum adjuvant boosts adaptive immunity by inducing uric acid and activating inflammatory dendritic cells,” J. Exp. Med., vol. 205, no. 4, pp. 869-882, April 2008.
[67] T. Marichal et al., “DNA released from dying host cells mediates aluminum adjuvant activity,” Nat. Med., vol. 17, no. 8, pp. 996-1002, August 2011.
[68] F. Ghiringhelli et al., “Activation of the NLRP3 inflammasome in dendritic cells induces IL-1B-dependent adaptive immunity against tumors,” Nat. Med., vol. 15, no. 10, pp. 1170-1178, 2009.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details can be made therein without departing from the scope of the invention encompassed by the appended claims.

1. A method of inducing or enhancing an adaptive immune response to a cancer in a subject, the method comprising:
administering an effective amount of (i) a Toll-Like Receptor (TLR) ligand; (ii) a non-canonical inflammasome-activating lipid; and (iii) a cancer immunogen to the subject.

2. A method of treating cancer in a subject, the method comprising:
administering an effective amount of (i) a Toll-Like Receptor (TLR) ligand; (ii) a non-canonical inflammasome-activating lipid and (iii) a cancer immunogen to the subject.

3. The method of claim 1, wherein the cancer immunogen is an infectious agent immunogen, wherein an infection with the infectious agent is associated with development of cancer.

4. The method of claim 1, wherein the cancer immunogen is from a cancer cell.

5. The method of claim 4, wherein the cancer immunogen is or comprises whole tumor cell lysate.

6. The method of claim 1, wherein the TLR ligand is selected from a TLR1 ligand, a TLR2 ligand, a TLR3 ligand, a TLR4 ligand, a TLR5 ligand, a TLR6 ligand, a TLR7 ligand, a TLR8 ligand, a TLR9 ligand, a TLR10 ligand, a TLR11 ligand, a TLR12 ligand, a TLR13 ligand, and combinations thereof.

7. The method of claim 1, wherein the TLR ligand is a TLR4 ligand.

8. The method of claim 7, wherein the TLR4 ligand is selected from monophosphoryl lipid A (MPLA), lipopolysaccharide (LPS), or combinations thereof.

9. The method of claim 1, wherein the non-canonical inflammasome-activating lipid comprises a species of oxidized 1-palmitoyl-2-arachidonoyl-sn-glycero-3-phosphorylcholine (oxPAPC).

10. The method of claim 1, wherein the non-canonical inflammasome-activating lipid comprises 2-[[(2R)-2-[(E)-7-carboxy-5-hydroxyhept-6-enoyl]oxy-3-hexadecanoyloxypropoxy]-hydroxyphosphoryl]oxyethyl-trimethylazanium (HOdiA-PC), [(2R)-2-[(E)-7-carboxy-5-oxohept-6-enoyl]oxy-3-hexadecanoyloxypropyl] 2-(trimethylazaniumyl)ethyl phosphate (KOdiA-PC), 1-palmitoyl-2-(5-hydroxy-8-oxo-octenoyl)-sn-glycero-3-phosphorylcholine (HOOA-PC), 2-[[(2R)-2-[(E)-5,8-dioxooct-6-enoyl]oxy-3-hexadecanoyloxypropoxy]-hydroxyphosphoryl]oxyethyl-trimethylazanium (KOOA-PC), [(2R)-3-hexadecanoyloxy-2-(5-oxopentanoyloxy)propyl] 2-(trimethylazaniumyl)ethyl phosphate (POVPC), [(2R)-2-(4-carboxybutanoyloxy)-3-hexadecanoyloxypropyl] 2-(trimethylazaniumyl)ethyl phosphate (PGPC), [(2R)-3-hexadecanoyloxy-2-[4-[3-[(E)-[2-[(Z)-oct-2-enyl]-5-oxocyclopent-3-en-1-ylidene]methyl]oxiran-2-yl]butanoyloxy]propyl] 2-(trimethylazaniumyl)ethyl phosphate (PECPC), [(2R)-3-hexadecanoyloxy-2-[4-[3-[(E)-[3-hydroxy-2-[(Z)-oct-2-enyl]-5-oxocyclopentylidene]methyl]oxiran-2-yl]butanoyloxy]propyl] 2-(trimethylazaniumyl)ethyl phosphate (PEIPC), or a combination thereof.

11. The method of claim 1, wherein the non-canonical inflammasome-activating lipid comprises [(2R)-2-(4-carboxybutanoyloxy)-3-hexadecanoyloxypropyl] 2-(trimethylazaniumyl)ethyl phosphate (PGPC).

12. The method of claim 1, wherein the subject is a mammal.

13. The method of claim 12, wherein the subject is a human.

14. The method of claim 1, wherein the TLR ligand, oxPAPC species, and cancer immunogen are administered as part of a pharmaceutical composition.

15. The method of claim 1, wherein the immune response is a prophylactic immune response.

16. The method of claim 1, wherein the immune response is a therapeutic immune response.

17. The method of claim 1, wherein the adaptive immune response comprises T-cell activation.

18. The method of claim 1 further comprising treating the subject with one or more therapeutic interventions.

19. The method of claim 1, wherein the TLR ligand, oxPAPC species, and cancer immunogen, and one or more therapeutic interventions are co-administered or sequentially administered.

20. The method of claim 18, wherein the one or more therapeutic interventions comprises: radiation, chemotherapy, surgery, therapeutic antibodies, immunomodulatory agents, proteasome inhibitors, pan-deacetylase (DAC) inhibitors, histone deacetylase (HDAC) inhibitors, checkpoint inhibitors, adoptive cell therapies, vaccines or combinations thereof.

21. The method of claim 21, wherein the adoptive cell therapies comprise: CAR-T cell therapy, CAR-NK cell therapy, T cells, dendritic cells or combinations thereof.
